(19) United States
(12) Patent Application Publication
(10) Pub. No.: US 2021/0170035 A1
(43) Pub. Date: Jun. 10, 2021

(54) METHOD OF TREATING CANCER BY TARGETING MYELOID-DERIVED SUPPRESSOR CELLS

(71) Applicants: Philip Stewart Low, West Lafayette, IN (US); Bingbing Wang, West Lafayette, IN (US)

(72) Inventors: Philip Stewart Low, West Lafayette, IN (US); Bingbing Wang, West Lafayette, IN (US)

(21) Appl. No.: 17/166,759

(22) Filed: Feb. 3, 2021

Related U.S. Application Data
(63) Continuation of application No. 17/156,251, filed on Jan. 22, 2021, now abandoned.
Application No. 17/156,251 is a continuation of application No. 16/302,912, filed on Nov. 19, 2018.
(60) Provisional application No. 62/341,587, filed on May 25, 2016.

(51) Int. Cl. A61K 47/55; A61K 38/07; A61K 45/06; A61K 31/5377; A61P 35/00; A61K 9/00; A61K 31/519

(52) U.S. Cl. CPC A61K 47/551; A61K 38/07; A61K 45/06; A61K 31/519; A61P 35/00; A61K 9/0019; A61K 31/5377

(57) ABSTRACT

The invention described herein relates to methods for treating a cancer using one or more compounds comprising a folate receptor binding ligand attached to a drug via a linker. More particularly, the invention described herein relates to methods for treating a cancer using one or more compounds comprising a folate receptor binding ligand attached to a drug via a linker to target myeloid-derived suppressor cells.

### CROSS REFERENCE TO RELATED APPLICATIONS

This application claims priority under 35 U.S.C. § 119(e) to U.S. Provisional Application Ser. No. 62/341,587, filed May 25, 2016, which is incorporated herein by reference in its entirety.

### FIELD OF THE DISCLOSURE

The invention described herein relates to methods for treating a cancer using one or more compounds comprising a folate receptor binding ligand attached to a drug via a linker. More particularly, the invention described herein relates to methods for a treating cancer using one or more compounds comprising a folate receptor binding ligand attached to a drug via a linker to target myeloid-derived suppressor cells.

### BACKGROUND AND SUMMARY

Despite the fact that there have been significant developments in anti-cancer technology, such as radiotherapy, chemotherapy and hormone therapy, cancer still remains the second leading cause of death following heart disease in the United States. Most often, cancer is treated with chemotherapy utilizing highly potent drugs, such as mitomycin, paclitaxel and camptothecin. In many cases, these chemotherapeutic agents show a dose responsive effect, and tumor inhibition is proportional to drug dose. Thus, an aggressive dosing regimen is used to treat neoplasms; however, high-dose chemotherapy is hindered by poor selectivity for cancer cells and toxicity to normal cells. A lack of tumor specificity is one of the many hurdles that need to be overcome by chemotherapies.

One solution to current chemotherapy limitations is to deliver an effective concentration of an anti-cancer agent with very high specificity. To reach this goal, much effort has been directed to developing tumor-selective drugs by conjugating anti-cancer drugs to hormones, antibodies, and vitamins. For example, the low molecular weight vitamin, folic acid, and other folate receptor binding ligands, are especially useful as targeting agents for folate receptor-positive cancers.

Folic acid is a member of the B family of vitamins and plays an essential role in cell survival by participating in the biosynthesis of nucleic and amino acids. This essential vitamin is also a high affinity ligand that enhances the specificity of conjugated anti-cancer drugs by targeting folate receptor-positive cancer cells. It has been found that the folate receptor (FR) is up-regulated in more than 90% of non-mucinous ovarian carcinomas. The folate receptor is also found at high to moderate levels in kidney, brain, lung, and breast carcinomas. In contrast, it has been reported that the folate receptor is present at low levels in most normal tissues leading to a mechanism for selectively targeting the cancer cells. Although the folate receptor can be used to deliver agents to tumor tissue with very high specificity, there are a number of cancers that do not express the folate receptor at all, or in sufficient numbers to provide the desired specificity. Thus, there is a need for developing therapies to treat such folate receptor-negative cancers.

Myeloid-derived supressor cells (MDSCs) are associated with tumors and can enhance immunosuppression in the tumor environment by suppressing such cells as T cells, NK cells, DC macrophages, and NKT cells. Thus, MDSCs can promote tumor growth, angiogenesis, and metastasis. The abundance of these cells in the tumor environment correlates negatively with cancer patient survival. Thus, therapies that deplete MDSCs would be useful.

Applicants have discovered that tumors that express the folate receptor, or that do not express the folate receptor in sufficient numbers, or at all, can be treated by targeting drugs to MDSCs because MDSCs express the folate receptor β. Thus, methods for treating cancers by targeting MDSCs using folate receptor binding ligands linked to a drug via a linker are described herein. MDSCs can be targeted using folate as the targeting ligand to deliver drugs to MDSCs to deplete or inhibit MDSCs and to treat a host animal with a cancer, whether or not the cancer expresses the folate receptor. Accordingly, it is to be understood that the methods described herein can be used to treat cancers that do not express the folate receptor, as well as cancers that do express the folate receptor.

In one embodiment, a method is provided for treating a folate receptor-negative cancer. The method comprises administering to the host animal a therapeutically effective amount of one or more compounds comprising a folate receptor binding ligand attached to a drug via a linker wherein myeloid-derived suppressor cells are inhibited or depleted.

In another embodiment, a method is provided for treating a folate receptor-negative cancer. The method comprises administering to the host animal a therapeutically effective amount of one or more compounds comprising a folate receptor binding ligand attached to a drug via a linker to deplete or inhibit myeloid-derived suppressor cells.

In yet another embodiment, a method is provided for treating a folate receptor-negative cancer in a host animal where myeloid-derived suppressor cells are in the cancer, the method comprising administering to the host animal a therapeutically effective amount of one or more compounds comprising a folate receptor binding ligand attached to a drug via a linker, and treating the cancer having the myeloid-derived suppressor cells.

In still another embodiment, a method is provided for treating a cancer. The method comprises identifying the presence of myeloid-derived suppressor cells in the cancer in a host animal, and administering to the host animal a therapeutically effective amount of one or more compounds comprising a folate receptor binding ligand attached to a drug via a linker.

In another illustrative embodiment, a method is provided for treating a cancer in a host animal. The method comprises administering to the host animal a therapeutically effective amount of one or more compounds comprising a folate receptor binding ligand attached to a drug via a linker to inhibit or deplete myeloid-derived suppressor cells.

In another embodiment, a method is provided for targeting myeloid-derived suppressor cells in a host animal. The method comprises administering to the host animal a therapeutically or diagnostically effective amount of one or more compounds comprising a folate receptor binding ligand attached to a drug via a linker to target the myeloid-derived suppressor cells.

Additional illustrative and non-limiting embodiments of the invention are described in the following enumerated clauses. All combinations of the following clauses are understood to be additional embodiments of the invention described herein. All applicable combinations of these embodiments with the embodiments described in the DETAILED DESCRIPTION OF THE ILLUSTRATIVE EMBODIMENTS section of the application are also embodiments of the invention.

1. A method for treating a folate receptor-negative cancer comprising administering to the host animal a therapeutically effective amount of one or more compounds comprising a folate receptor binding ligand attached to a drug via a linker wherein myeloid-derived suppressor cells are inhibited or depleted.

2. A method for treating a folate receptor-negative cancer comprising administering to the host animal a therapeutically effective amount of one or more compounds comprising a folate receptor binding ligand attached to a drug via a linker to deplete or inhibit myeloid-derived suppressor cells.

3. A method for treating a folate receptor-negative cancer in a host animal where myeloid-derived suppressor cells are in the cancer, the method comprising administering to the host animal a therapeutically effective amount of one or more compounds comprising a folate receptor binding ligand attached to a drug via a linker, and treating the cancer having the myeloid-derived suppressor cells.

4. A method for treating a cancer comprising identifying the presence of myeloid-derived suppressor cells in the cancer in a host animal, and administering to the host animal a therapeutically effective amount of one or more compounds comprising a folate receptor binding ligand attached to a drug via a linker.

5. A method for treating a cancer in a host animal, the method comprising administering to the host animal a therapeutically effective amount of one or more compounds comprising a folate receptor binding ligand attached to a drug via a linker to inhibit or deplete myeloid-derived suppressor cells.

6. A method for targeting myeloid-derived suppressor cells in a host animal, the method comprising administering to the host animal a therapeutically or diagnostically effective amount of one or more compounds comprising a folate receptor binding ligand attached to a drug via a linker to target the myeloid-derived suppressor cells.

7. The method of any one of clauses 4 to 6 wherein the cancer is folate receptor-negative.

8. The method of any one of clauses 4 to 6 wherein the cancer is folate receptor-positive.

9. The method of any one of clauses 1 to 8 wherein the folate receptor binding ligand is specific for folate receptor β and wherein the folate receptor binding ligand binds to the folate receptor β on the myeloid-derived suppressor cells.

10. The method of any one of clauses 1 to 9 wherein the myeloid-derived suppressor cells have a CD11b marker.

11. The method of any one of clauses 1 to 10 wherein the myeloid-derived suppressor cells have a Gr1 marker.

12. The method of any one of clauses 1 to 11 wherein the cancer is selected from non-small cell lung cancer, head and neck cancer, triple negative breast cancer, breast cancer, ovarian cancer, colon cancer, prostate cancer, lung cancer, endometrial cancer, and renal cancer.

13. The method of any one of clauses 1 to 12 wherein the drug is selected from CI307, BEZ235, wortmannin, AMT, PF-04691502, a CpG oligonucleotide, BLZ945, lenalidomide, NLG919, 5,15-DPP, a pyrrolobenzodiazepine, methotrexate, everolimus, a tubulysin, GDC-0980, AS1517499, BIRB796, n-acetyl-5-hydroxytryptamine, and 2,4-diamino-6-hydroxpyrimidine.

14. The method of any one of clauses 1 to 13 wherein the drug is a microtubule inhibitor.

15. The method of clause 14 wherein the drug kills myeloid-derived suppressor cells.

16. The method of any one of clauses 1 to 13 wherein the drug is selected from a PI3K inhibitor, a STAT6 inhibitor, a MAPK inhibitor, an iNOS inhibitor, and an anti-inflammatory drug.

17. The method of clause 16 wherein the drug inactivates myeloid-derived suppressor cells.

18. The method of any one of clauses 1 to 13 wherein the drug is a TLR agonist.

19. The method of clause 18 wherein the TLR agonist is selected from a TLR7 agonist and a TLR 9 agonist.

20. The method of clause 18 or 19 wherein the drug reprograms myeloid-derived suppressor cells.

21. The method of clause 14 or 15 wherein the drug is a tubulysin.

22. The method of clause 16 wherein the drug is a PI3K inhibitor.

23. The method of clause 22 wherein the drug is selected from GDC-0980, wortmannin, and PF-04691502.

24. The method of clause 16 wherein the drug is a STAT6 inhibitor.

25. The method of clause 24 wherein the drug is AS1517499.

26. The method of clause 16 wherein the drug is a MAPK inhibitor.

27. The method of clause 26 wherein the drug is BIRB796.

28. The method of clause 16 wherein the drug is an iNOS inhibitor.

29. The method of clause 28 wherein the drug is AMT.

30. The method of clause 16 wherein the drug is an anti-inflammatory drug.

31. The method of clause 30 wherein the drug is methotrexate.

32. The method of any one of clauses 18 to 20 wherein the drug is selected from CI307, a CpG oligonucleotide, and TLR7A.

33. The method of any one of clauses 1 to 13 wherein more than one compound is administered and the compounds comprise different drugs.

34. The method of claim 33 wherein the different drugs are a TLR7 agonist and a PI3K inhibitor.

35. The method of any one of clauses 1 to 32 wherein one or more compound is administered and an unconjugated drug is also administered.

36. The method of clause 35 wherein the drug in the compound is a TLR7 agonist and the unconjugated drug is a PI3K inhibitor.

37. The method of any one of clauses 1 to 12, where the compound is of the formula

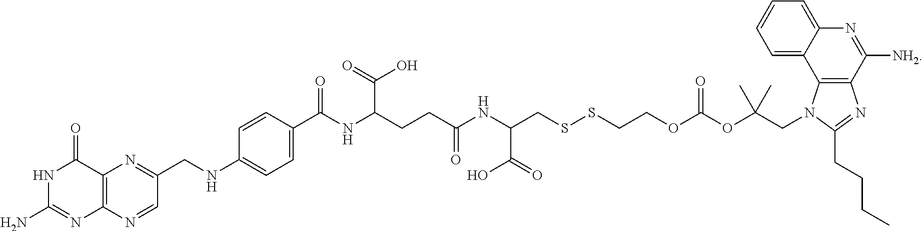 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

38. The method of any one of clauses 1 to 12, where the compound is of the formula

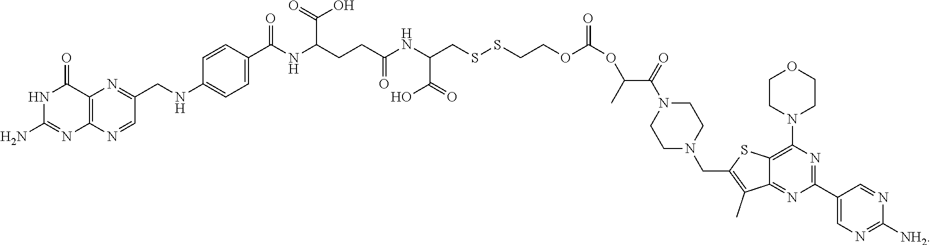 [SMILES: Cc1c(CN2CCN(C(=O)C(C)OC(=O)OCCSSCC(NC(=O)CCC(NC(=O)c3ccc(NCc4cnc5nc(N)[nH]c(=O)c5n4)cc3)C(=O)O)C(=O)O)CC2)sc2c(N3CCOCC3)nc(-c3cnc(N)nc3)nc12]

39. The method of any one of clauses 1 to 12, where the compound is of the formula

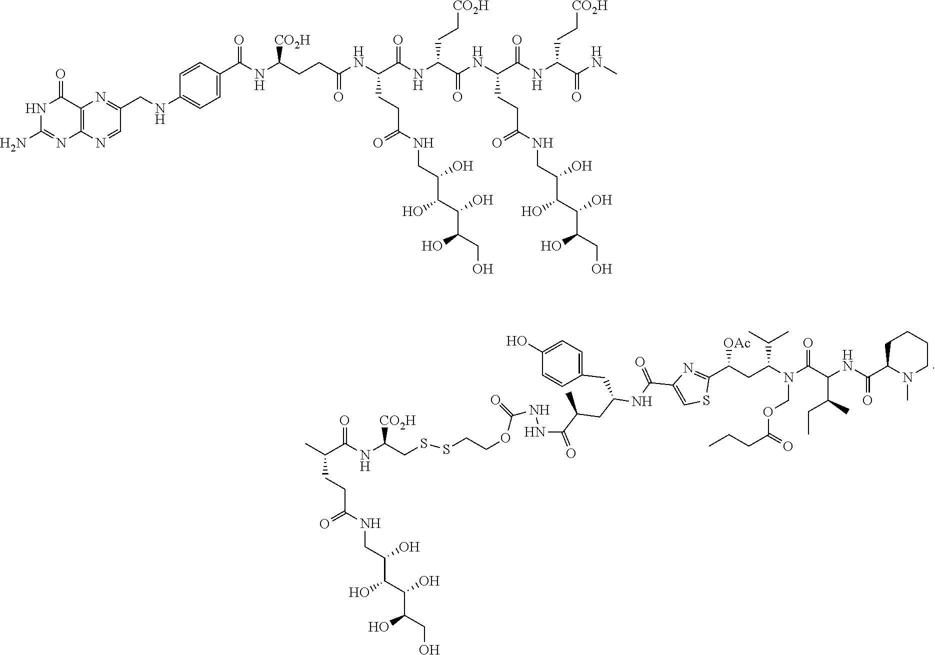

40. The method of any one of clauses 1 to 12, where the compound is of the formula

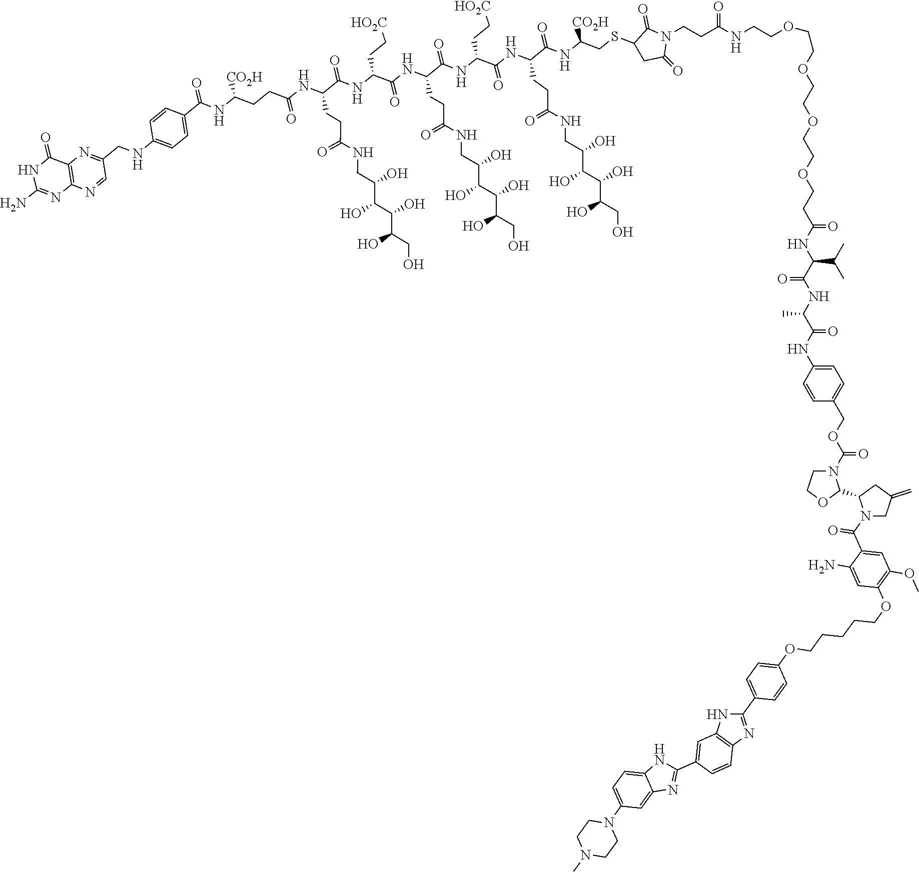 [SMILES: C=C1C[C@@H](C2OCCN2C(=O)OCc2ccc(NC(=O)[C@H](C)NC(=O)[C@@H](NC(=O)CCOCCOCCOCCOCCNC(=O)CCN3C(=O)CC(SC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@H](NC(=O)c4ccc(NCc5cnc6nc(N)[nH]c(=O)c6n5)cc4)C(=O)O)C(=O)O)C3=O)C(C)C)cc2)N(C(=O)c2cc(OC)c(OCCCCCOc3ccc(-c4nc5ccc(-c6nc7cc(N8CCN(C)CC8)ccc7[nH]6)cc5[nH]4)cc3)cc2N)C1]

41. The method of any one of clauses 1 to 40 wherein the one or more compounds, or a pharmaceutically acceptable salt of any of the one or more compounds, is administered to the host animal.

42. The method of any one of clauses 1 to 41 wherein the administration is in a parenteral dosage form.

43. The method of clause 42 wherein the parenteral dosage form is selected from an intradermal dosage form, a subcutaneous dosage form, an intramuscular dosage form, an intraperitoneal dosage form, an intravenous dosage form, and an intrathecal dosage form.

44. The method of any one of clauses 1 to 43 wherein the therapeutically effective amount or the diagnostically effective amount is from about 0.5 mg/m² to about 6.0 mg/m².

45. The method of any one of clauses 1 to 44 wherein the therapeutically effective amount or the diagnostically effective amount is from about 0.5 mg/m² to about 4.0 mg/m².

46. The method of any one of clauses 1 to 45 wherein the therapeutically effective amount or the diagnostically effective amount is from about 0.5 mg/m² to about 2.0 mg/m².

47. The method of any one of clauses 1 to 7 or 9 to 46 wherein the cancer is folate receptor-negative and the cancer is selected from colon cancer, lung cancer, prostate cancer, and breast cancer.

### BRIEF DESCRIPTION OF THE DRAWINGS

FIG. 1 shows hematoxylin and eosin staining of FR-α expression on various human tumors: liver cancer (FIG. 1a); head & neck cancer (FIG. 1b); Thymoma (FIG. 1c).

FIG. 2 shows hematoxylin and eosin staining of FR-β expression on various human tumors: liver cancer (FIG. 2a); head & neck cancer (FIG. 2b); Thymoma (FIG. 2c).

FIG. 3 shows hematoxylin and eosin staining of FR-β expression on various human tumors: bladder cancer (FIG. 3a); brain cancer (FIG. 3b); liver cancer (FIG. 3c).

FIG. 4 shows hematoxylin and eosin staining of FR-β expression on various human tumors: renal cancer (FIG. 4a); skin cancer (FIG. 4b); thymus carcinoma (FIG. 4c).

FIG. 5 shows FR-β expression on mouse MDSCs (CD11b+Gr1+). FIG. 5a: MDSCs population gated on live cells; FIG. 5b: FR-β expression on gated MDSC population.

FIG. 6 shows FR-β expression on mouse TAMs (CD11b+F4/80). FIG. 6a: TAMs population gated on live cells; FIG. 6b: FR-β expression on gated TAM population.

FIG. 7 shows in vitro arginase production by TAMs/MDSCs after co-culture with various drugs. FIG. 7a: (●) CL307; (▪) BEZ235; (▴) Wortmannin; (▾) AMT. FIG. 7b: (♦) CpG; (∘) BIZ945; (□) Lenalidomide; (Δ) NLG919. FIG. 7c: ( [symbol] ) N-acetyl-5-hydroxyptamine; (⋄) 2,4-diamino-6-hydroxypyritnidine; (▪) 5,15-DPP; (x) methotrexate. FIG. 7d: (+) everolemus; ( [symbol] ) tubulysin; ( [symbol] ) AS1517499; ( [symbol] ) BIRB796 (doramapinod).

FIG. 8 shows in vitro IL-10 production by TAMs/MDSCs after co-culture with various drugs. FIG. 8a: (▪) BEZ235; (▴) Wortmannin; (▾) AMT. FIG. 8b: (∘) BIZ945; (□) Lenalidomide; (Δ) NLG919. FIG. 8c: ( [symbol] ) N-acetyl-5-hydroxyptamine; (⋄) 2,4-diamino-6-hydroxypyrimidine; (▪) 5,15-DPP; (x) methotrexate. FIG. 8d: (|) everolemus; ( [symbol] ) tubulysin; ( [symbol] ) AS1517499; ( [symbol] ) BIRB796 (doramapinod).

FIG. 9. shows in vitro nitric oxide production by TAMs/MDSCs after co-culture with various drugs. FIG. 9a: (▪) BEZ235; (▴) Wortmannin; (▾) AMT. FIG. 9b: (∘) BIZ945; (□) Lenalidomide; (Δ) NLG919. FIG. 9c: ( [symbol] ) N-acetyl-5-hydroxyptamine; (⋄) 2,4-diamino-6-hydroxypyrimidine; (●) 5,15-DPP; (x) methotrexate. FIG. 9d: (+) everolemus; ( [symbol] ) tubulysin; ( [symbol] ) AS1517499; ( [symbol] ) BIRB796 (doramapinod).

FIG. 10. shows in FIG. 10a, nitric oxide production by TAMs/MDSCs after co-culture with two TLR agonists, (●) CpG (TLR9 agonist) and (♦) CL307 (TLR7 agonist), at different concentrations. The black dotted line indicates the nitric oxide level from untreated control; FIG. 10b, CD86 expression on MDSCs as measured by flow cytometry after co-culture with different TLR agonists: resiquimod (TLR7/8 agonist), CpG ODN (TLR9 agonist), Poly IC (TLR3 agonist), zymosan (TLR2 agonist).

FIG. 11 shows Arginase (FIG. 11a) and nitric oxide (FIG. 11b) production by two TLR7 agonists, (▪) CL307 and (●) TLR7A, tested in vitro by co-culturing TAMs/MDSCs with different concentrations of the two drugs. The black dotted line in FIG. 11a indicated arginase level in untreated control. Black solid line in FIG. 11a indicate the arginase level of the background.

FIG. 12 shows arginase production by TAMs/MDSCs after co-culture with three PI3K inhibitors (BEZ235, PF-04691502 and GDC-0980) to identify the PI3K inhibitor activity to efficiently suppress TAMs/MDSCs function.

FIG. 13 shows IL-10 production by TAMs/MDSCs after co-culture with three PI3K inhibitors (BEZ235, PF-04691502 and GDC-0980) to identify the PI3K inhibitor activity to efficiently suppress TAMs/MDSCs function.

FIG. 14. shows nitric oxide production by TAMs/MDSCs after co-culture with three PI3K inhibitors (BEZ235, PF-04691502 and GDC-0980) to identify the PI3K inhibitor activity to efficiently suppress TAMs/MDSCs function.

FIG. 15. shows a synergistic curve of arginase production by in vitro combination treatment of TAMs/MDSCs with the TLR7 agonist (CL307) and the PI3K inhibitor (BEZ235); (▪) single treatment, (●) combination treatment.

FIG. 16 shows a dose study of FA-TLR7 agonist (FA-TLR7A) in a 4T1 solid tumor model. FIG. 16a shows tumor growth from groups of untreated control (●), 2 nmol treatment (▪) and 5 nmol (triangle) treatment. FIG. 16b shows tumor growth from groups of untreated control (●), 10 nmol (▾) treatment and 20 nmol (♦) treatment.

FIG. 17 shows animal weights for different groups of the dose study in the 4T1 solid tumor model shown in FIG. 16. Weights were measured every day from starting treatment at day 6. FIG. 17a shows weights from groups of untreated control (●), 2 nmol treatment (▪) and 5 nmol (triangle) treatment. FIG. 17b shows weights from groups of untreated control (●), 10 nmol (▾) treatment and 20 nmol (♦) treatment.

FIG. 18 shows an in vivo therapeutic study of FA-TLR7 agonist in a 4T1 solid tumor model. FIG. 18a shows tumor growth as measured every day after treatment started, (●) untreated control, (▪) FA-TLR7 agonist, (∘) competition-FA-TLR7 agonist. FIG. 18b shows animal weight as measured every day after treatment started, (●) untreated control, (▪) FA-TLR7 agonist, (∘) competition-FA-TLR7 agonist.

FIG. 19. shows an in vivo therapeutic study of FA-tubulysin in a 4T1 solid tumor model. FIG. 19a shows tumor growth as measured every day after treatment started, (●) untreated control, (▴) FA-tubulysin, (□) competition-FA-tubulysin. FIG. 19b shows animal weight as measured every day after treatment started, (●) untreated control, (▴) FA-tubulysin, (□) competition-FA-tubulysin.

FIG. 20 shows an in vivo therapeutic study of FA-PI3K inhibitor in a 4T1 solid tumor model. FIG. 20a shows tumor growth as measured every day after treatment started, (●) untreated control, (▾) FA-PI3K inhibitor, (Δ) competition-FA-PI3K inhibitor. FIG. 20b shows animal weight as measured every day after treatment started, (●) untreated control, (▾) FA-PI3K inhibitor, (Δ) competition-FA-PI3K inhibitor.

FIG. 21 shows an in vivo therapeutic study of combination treatment with FA-TLR7 agonist and non-targeted PI3K inhibitor (BEZ235) in a 4T1 solid tumor model. FIG. 21a shows tumor growth as measured every day after treatment started, (●) untreated control, (♦) combination, ( [symbol] ) competition-combination. FIG. 21b shows animal weight as measured every day after treatment started, (●) untreated control, (♦) combination, ( [symbol] ) competition-combination.

FIG. 22 shows an in vivo therapeutic study of FA-TLR7 agonist and non-targeted PI3K inhibitor (BEZ235) in a 4T1 solid tumor model. FIG. 22a shows tumor growth as measured every day after treatment started, (●) untreated control, (▪) FA-TLR7 agonist, (⋄) PI3K inhibitor. FIG. 22b shows animal weight as measured every day after treatment started, (●) untreated control, (▪) FA-TLR7 agonist, (⋄) PI3K inhibitor.

FIG. 23 shows average tumor volume at the last day of treatment for a therapeutic group for each of untreated control, FA-TLR7 agonist, FA-tubulysin, FA-PI3K inhibitor and a combination of FA-TLR7 agonist and non-targeted PI3K inhibitor (BEZ235). * and *** indicate statistically significant results.

FIG. 24 shows intracellular staining of arginase on F4/80+ macrophages was tested in groups of untreated control, FA-TLR7 agonist (FIG. 24a), FA-PI3K inhibitor (FIG. 24c), FA-Tubulysin (FIG. 24b), and combination (FIG. 24d) as well as competition groups. * indicates statistically significant results, ns indicates not statistically significant results.

FIG. 25 shows the ratio of M1 to M2 macrophages (F4/80+CD86+: F4/80+CD206+) tested in groups of untreated control, FA-TLR7 agonist (FIG. 25a), FA-PI3K inhibitor (FIG. 25c), FA-Tubulysin (FIG. 25b) and combination (FIG. 25d) as well as competition groups. * indicates statistically significant results, ns indicates not statistically significant results.

FIG. 26 shows MDSCs population (CD11b+Gr1+) tested in groups of untreated control, FA-TLR7 agonist (FIG. 26a), FA-PI3K inhibitor (FIG. 26c), FA-Tubulysin (FIG. 26b) and combination (FIG. 26d) as well as competition groups. * indicates statistically significant results, ns indicates not statistically significant results.

FIG. 27 shows percentages of CD4 (FIG. 27a) and CD8 (FIG. 27b) T cell populations tested in live cells isolated from 4T1 solid tumors in groups of untreated control, FA-TLR7 agonist, FA-PI3K inhibitor, FA-Tubulysin, and combination groups.

FIG. 28 shows in vitro induced human MDSCs responded to selected drugs by decreasing IL-10 production. (●) vinblastine; (▪) GDC0980; (▾) BEZ235; (♦) tubulysin.

FIG. 29 A-B show inhibition of human T cell suppression by MDSCs after being treated with 3 classes of drugs. FIG. 29A shows results after being treated with drugs at 0.1 μM of drug; FIG. 29B shows results after being treated with drugs at 1.0 μM of drug.

FIG. 30 A-C show resistance of 4T1 cells to three classes of drugs. 4T1 cells were cultured with 3 drugs for 36 hours. The cytotoxicity was evaluated by LDH assay. FIG. 30A shows results for TLR agonist at various concentrations; FIG. 30B shows results for PI3K inhibitor at various concentrations; FIG. 30C shows results for tubulysin at various concentrations.

FIG. 31 A-C show resistance of 4T1 cells to three classes of FA-conjugates. 4T1 cells were cultured with FA-conjugates for 3 hours. The cells were washed with PBS and incubated with medium for 36 hours. FIG. 31A shows results for TLR agonist conjugate at various concentrations; FIG. 31B shows results for PI3K inhibitor conjugate at various concentrations; FIG. 31C shows results for tubulysin conjugate at various concentrations.

FIG. 32. Tumor growth of 4T1 by continuous treatment with FA-conjugates for 2 weeks. (●) Control Mouse 1; (▪) Control Mouse 2; (▴) Control Mouse 3; (∘) FA-PI3K inhibitor conjugate Mouse 1; (□) FA-PI3K inhibitor conjugate Mouse 2; (Δ) FA-PI3K inhibitor conjugate Mouse 3; (⊙) FA-TLR7 agonist Mouse 1; ( [symbol] ) FA-TLR7 agonist Mouse 2; (⊗) FA-TLR7 agonist Mouse 3.

FIG. 33 shows arginase levels measured in MDSCs and TAMs from 4T1 tumor after 2 weeks continuous treatment with folate drug conjugates. ( [symbol] ) MDSC; ( [symbol] ) TAMs.

FIG. 34 shows lung metastasis evaluation in Balb/c mice with 4T1 solid tumor that were treated with three classes of FA-conjugates for 2 weeks (7 days/week). Lung was removed at the end of the study and metastasis was evaluated following standard procedures described in Example 15.

FIG. 35 shows a summary of lung metastasis in a 4T1 tumor model by targeting MDSCs/TAMs.

FIG. 36 shows monitoring of tumor growth survival study: Tumor volume was monitored in 4T1 a survival study of three folate-drug conjugates until surgically removing tumor at day 5. (●) Control; (∘) FA-TLR7 agonist conjugate; (Δ) FA-PI3K inhibitor conjugate; (□) FA-tubulysin conjugate.

FIG. 37 shows survival curve of mice with 4T1 solid tumor (n=2 for FA-TLR7 agonist, n=3 for FA-PI3K inhibitor and disease control, n=4 for FA-tubulysin). (▪) Control; (Δ) FA-TLR7 agonist conjugate; (∘) FA-PI3K inhibitor conjugate; (□) FA-tubulysin conjugate. The 41-day time point at 100% includes all symbols except the symbol for the control.

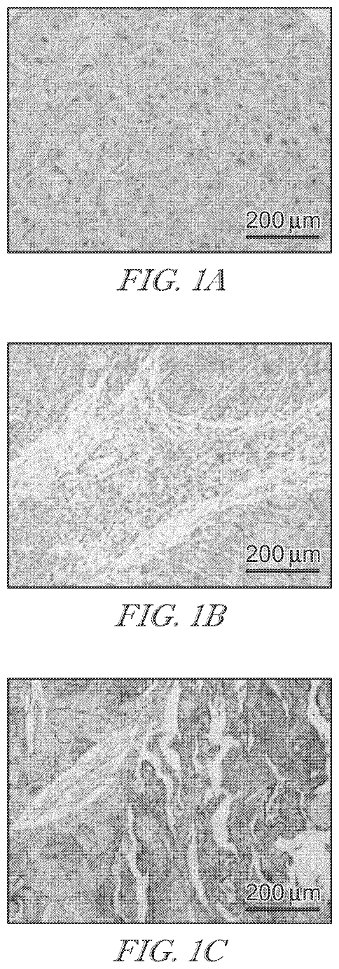

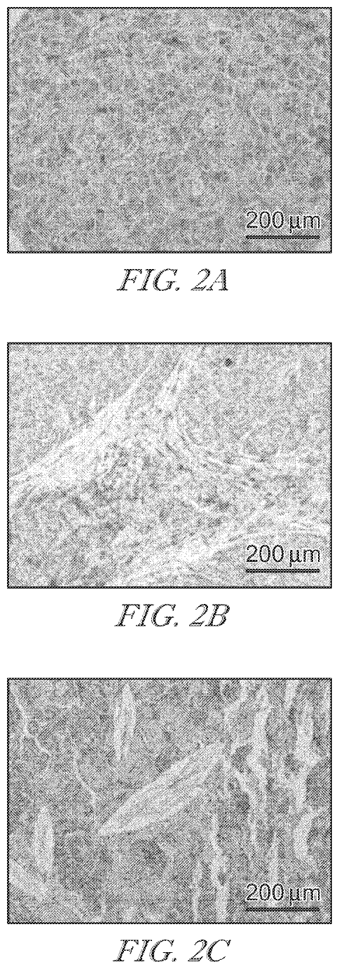

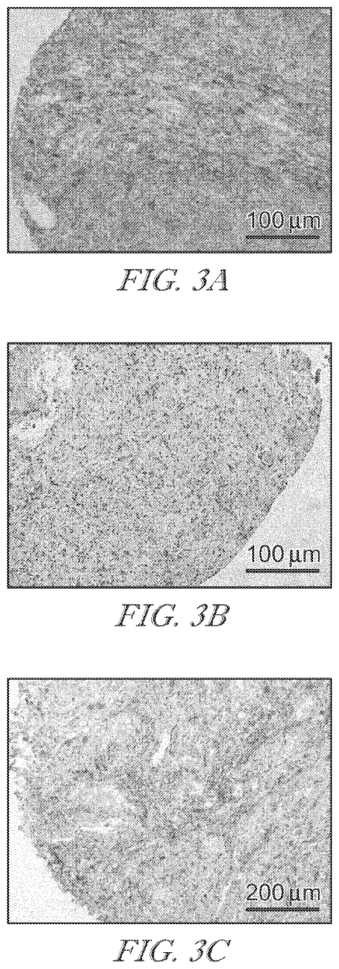

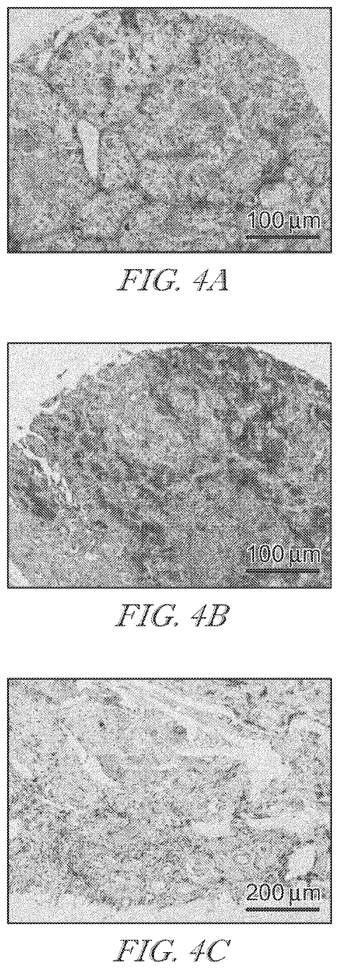

### DETAILED DESCRIPTION OF ILLUSTRATIVE EMBODIMENTS

It is to be understood that each embodiment of the invention described herein may be, as applicable, combined with any other embodiment described herein. For example, any of the embodiments in the Summary, and/or of the enumerated clauses described herein, or any applicable combination thereof, may be combined with any of the embodiments described in the Detailed Description of Illustrative Embodiments section of this patent application. As used herein, the term “myeloid-derived suppressor cells” (MDSCs) refers to cells that exist in the microenvironment of a cancer, for example, a tumor, are immunosuppressive, and have one or more of the markers CD11b and Gr1. MDSCs can be identified by methods known in the art, for example, by flow cytometry using markers specific for MDSCs, such as CD11b and Gr1.

As used herein, the phrase “wherein myeloid-derived suppressor cells are in the cancer” generally refers to MDSCs that exist in the microenvironment of a cancer (e.g., a tumor), or, for example, are found in cancerous tissue (e.g., tumor tissue).

As used herein, the term “administering” generally refers to any and all means of introducing compounds described herein to the host animal, including, but not limited to, by oral (po), intravenous (iv), intramuscular (im), subcutaneous (sc), transdermal, inhalation, buccal, ocular, sublingual, vaginal, rectal, and like routes of administration. Compounds described herein may be administered in unit dosage forms and/or compositions containing one or more pharmaceutically-acceptable carriers, adjuvants, diluents, excipients, and/or vehicles, and combinations thereof.

As used herein, the term “composition” generally refers to any product comprising more than one ingredient, including the compounds described herein. It is to be understood that the compositions described herein may be prepared from isolated compounds described herein or from salts, solutions, hydrates, solvates, and other forms of the compounds described herein. It is appreciated that certain functional groups, such as the hydroxy, amino, and like groups may form complexes with water and/or various solvents, in the various physical forms of the compounds. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, or individual forms of, the various morphological forms and/or solvate or hydrate forms of the compounds described herein.

Applicants have discovered that tumors that express the folate receptor, or that do not express the folate receptor in sufficient numbers, or at all, can be treated by targeting drugs to MDSCs because MDSCs express the folate receptor β. Thus, methods for treating cancers by targeting MDSCs using folate receptor binding ligands linked to a drug via a linker are described herein. MDSCs can be targeted using folate as the targeting ligand to deliver drugs to MDSCs to deplete or inhibit MDSCs and to treat a host animal with a cancer, whether or not the cancer expresses the folate receptor. Accordingly, it is to be understood that the methods described herein can be used to treat cancers that do not express the folate receptor, as well as cancers that do express the folate receptor.

In one embodiment, a method is provided for treating a folate receptor-negative cancer. The method comprises administering to the host animal a therapeutically effective amount of one or more compounds comprising a folate receptor binding ligand attached to a drug via a linker wherein myeloid-derived suppressor cells are inhibited or depleted.

In another embodiment, a method is provided for treating a folate receptor-negative cancer. The method comprises administering to the host animal a therapeutically effective amount of one or more compounds comprising a folate receptor binding ligand attached to a drug via a linker to deplete or inhibit myeloid-derived suppressor cells.

In yet another embodiment, a method is provided for treating a folate receptor-negative cancer in a host animal where myeloid-derived suppressor cells are in the cancer, the method comprising administering to the host animal a therapeutically effective amount of one or more compounds comprising a folate receptor binding ligand attached to a drug via a linker, and treating the folate receptor negative cancer having the myeloid-derived suppressor cells.

In still another embodiment, a method is provided for treating a cancer. The method comprises identifying the presence of myeloid-derived suppressor cells in the cancer in a host animal, and administering to the host animal a therapeutically effective amount of one or more compounds comprising a folate receptor binding ligand attached to a drug via a linker.

In another illustrative embodiment, a method is provided for treating a cancer in a host animal. The method comprises administering to the host animal a therapeutically effective amount of one or more compounds comprising a folate receptor binding ligand attached to a drug via a linker to inhibit or deplete myeloid-derived suppressor cells.

In another embodiment, a method is provided for targeting myeloid-derived suppressor cells in a host animal. The method comprises administering to the host animal a therapeutically or diagnostically effective amount of one or more compounds comprising a folate receptor binding ligand attached to a drug via a linker to target the myeloid-derived suppressor cells.

Additional illustrative and non-limiting embodiments of the invention are described in the following enumerated clauses.

1. A method for treating a folate receptor-negative cancer comprising administering to the host animal a therapeutically effective amount of one or more compounds comprising a folate receptor binding ligand attached to a drug via a linker wherein myeloid-derived suppressor cells are inhibited or depleted.

2. A method for treating a folate receptor-negative cancer comprising administering to the host animal a therapeutically effective amount of one or more compounds comprising a folate receptor binding ligand attached to a drug via a linker to deplete or inhibit myeloid-derived suppressor cells.

3. A method for treating a folate receptor-negative cancer in a host animal where myeloid-derived suppressor cells are in the cancer, the method comprising administering to the host animal a therapeutically effective amount of one or more compounds comprising a folate receptor binding ligand attached to a drug via a linker, and treating the cancer having the myeloid-derived suppressor cells.

4. A method for treating a cancer comprising identifying the presence of myeloid-derived suppressor cells in the cancer in a host animal, and administering to the host animal a therapeutically effective amount of one or more compounds comprising a folate receptor binding ligand attached to a drug via a linker.

5. A method for treating a cancer in a host animal, the method comprising administering to the host animal a therapeutically effective amount of one or more compounds comprising a folate receptor binding ligand attached to a drug via a linker to inhibit or deplete myeloid-derived suppressor cells.

6. A method for targeting myeloid-derived suppressor cells in a host animal, the method comprising administering to the host animal a therapeutically or diagnostically effective amount of one or more compounds comprising a folate receptor binding ligand attached to a drug via a linker to target the myeloid-derived suppressor cells.

7. The method of any one of clauses 4 to 6 wherein the cancer is folate receptor-negative.

8. The method of any one of clauses 4 to 6 wherein the cancer is folate receptor-positive.

9. The method of any one of clauses 1 to 8 wherein the folate receptor binding ligand is specific for folate receptor β and wherein the folate receptor binding ligand binds to the folate receptor β on the myeloid-derived suppressor cells.

10. The method of any one of clauses 1 to 9 wherein the myeloid-derived suppressor cells have a CD11b marker.

11. The method of any one of clauses 1 to 10 wherein the myeloid-derived suppressor cells have a Gr1 marker.

12. The method of any one of clauses 1 to 11 wherein the cancer is selected from non-small cell lung cancer, head and neck cancer, triple negative breast cancer, breast cancer, ovarian cancer, colon cancer, prostate cancer, lung cancer, endometrial cancer, and renal cancer.

13. The method of any one of clauses 1 to 12 wherein the drug is selected from CI307, BEZ235, wortmannin, AMT, PF-04691502, a CpG oligonucleotide, BLZ945, lenalidomide, NLG919, 5,15-DPP, a pyrrolobenzodiazepine, methotrexate, everolimus, a tubulysin, GDC-0980, AS1517499, BIRB796, n-acetyl-5-hydroxytryptamine, and 2,4-diamino-6-hydroxpyrimidine.

14. The method of any one of clauses 1 to 13 wherein the drug is a microtubule inhibitor.

15. The method of clause 14 wherein the drug kills myeloid-derived suppressor cells.

16. The method of any one of clauses 1 to 13 wherein the drug is selected from a PI3K inhibitor, a STAT6 inhibitor, a MAPK inhibitor, an iNOS inhibitor, and an anti-inflammatory drug.

17. The method of clause 16 wherein the drug inactivates myeloid-derived suppressor cells.

18. The method of any one of clauses 1 to 13 wherein the drug is a TLR agonist.

19. The method of clause 18 wherein the TLR agonist is selected from a TLR7 agonist and a TLR 9 agonist.

20. The method of clause 18 or 19 wherein the drug reprograms myeloid-derived suppressor cells.

21. The method of clause 14 or 15 wherein the drug is a tubulysin.

22. The method of clause 16 wherein the drug is a PI3K inhibitor.

23. The method of clause 22 wherein the drug is selected from GDC-0980, wortmannin, and PF-04691502.

24. The method of clause 16 wherein the drug is a STAT6 inhibitor.

25. The method of clause 24 wherein the drug is AS1517499.

26. The method of clause 16 wherein the drug is a MAPK inhibitor.

27. The method of clause 26 wherein the drug is BIRB796.

28. The method of clause 16 wherein the drug is an iNOS inhibitor.

29. The method of clause 28 wherein the drug is AMT.

30. The method of clause 16 wherein the drug is an anti-inflammatory drug.

31. The method of clause 30 wherein the drug is methotrexate.

32. The method of any one of clauses 18 to 20 wherein the drug is selected from CI307, a CpG oligonucleotide, and TLR7A.

33. The method of any one of clauses 1 to 13 wherein more than one compound is administered and the compounds comprise different drugs.

34. The method of claim 33 wherein the different drugs are a TLR7 agonist and a PI3K inhibitor.

35. The method of any one of clauses 1 to 32 wherein one or more compound is administered and an unconjugated drug is also administered.

36. The method of clause 35 wherein the drug in the compound is a TLR7 agonist and the unconjugated drug is a PI3K inhibitor.

37. The method of any one of clauses 1 to 12, where the compound is of the formula

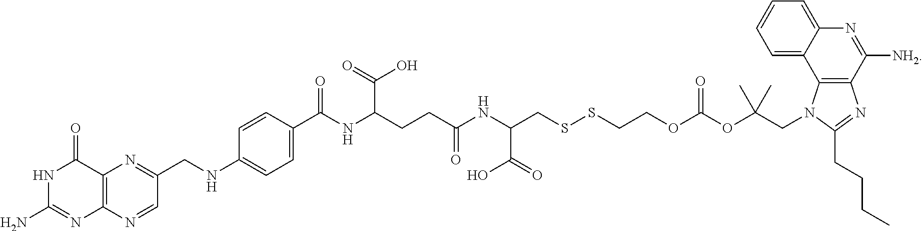 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

38. The method of any one of clauses 1 to 12, where the compound is of the formula

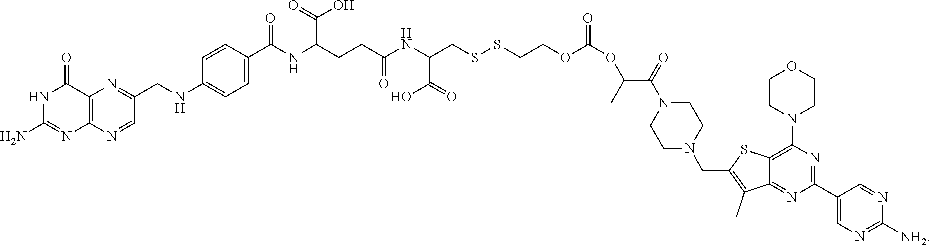 [SMILES: Cc1c(CN2CCN(C(=O)C(C)OC(=O)OCCSSCC(NC(=O)CCC(NC(=O)c3ccc(NCc4cnc5nc(N)[nH]c(=O)c5n4)cc3)C(=O)O)C(=O)O)CC2)sc2c(N3CCOCC3)nc(-c3cnc(N)nc3)nc12]

39. The method of any one of clauses 1 to 12, where the compound is of the formula

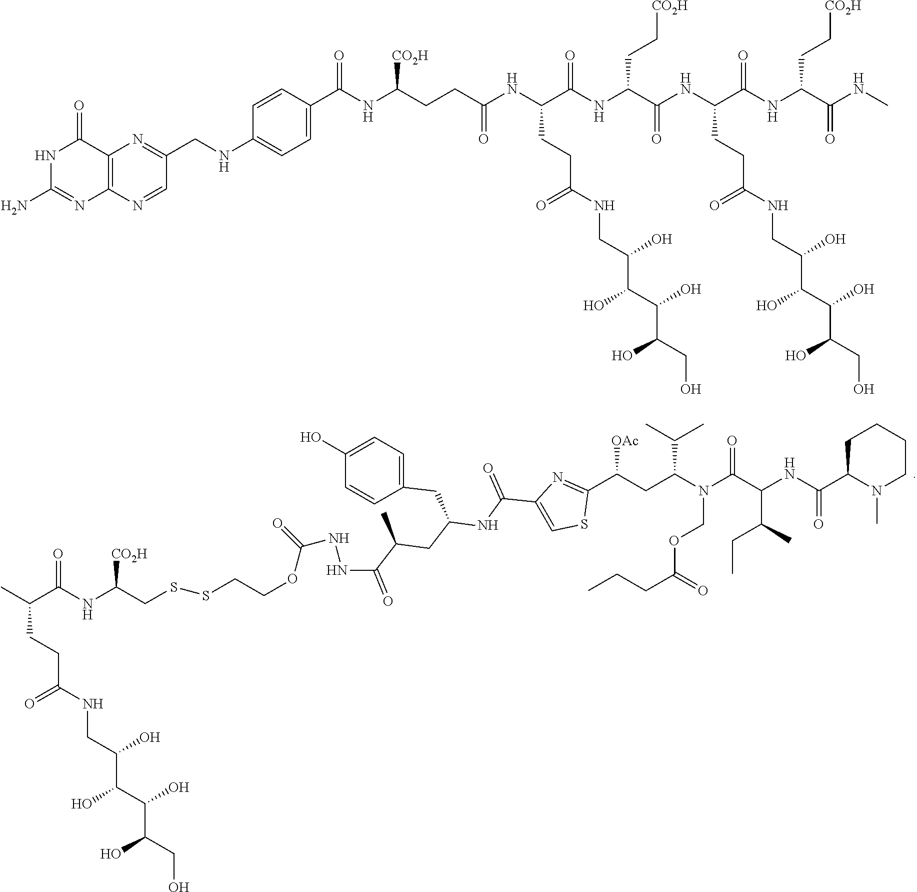

40. The method of any one of clauses 1 to 12, where the compound is of the formula

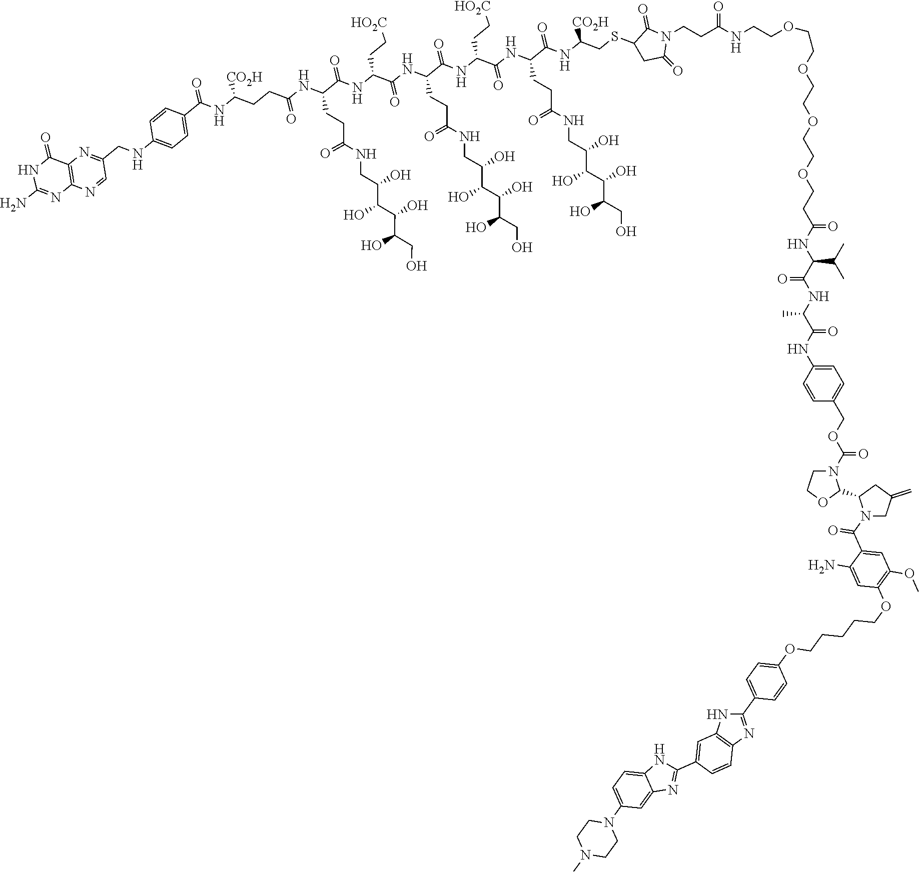 [SMILES: C=C1C[C@@H](C2OCCN2C(=O)OCc2ccc(NC(=O)[C@H](C)NC(=O)[C@@H](NC(=O)CCOCCOCCOCCOCCNC(=O)CCN3C(=O)CC(SC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@H](NC(=O)c4ccc(NCc5cnc6nc(N)[nH]c(=O)c6n5)cc4)C(=O)O)C(=O)O)C3=O)C(C)C)cc2)N(C(=O)c2cc(OC)c(OCCCCCOc3ccc(-c4nc5ccc(-c6nc7cc(N8CCN(C)CC8)ccc7[nH]6)cc5[nH]4)cc3)cc2N)C1]

41. The method of any one of clauses 1 to 40 wherein the one or more compounds, or a pharmaceutically acceptable salt of any of the one or more compounds, is administered to the host animal.

42. The method of any one of clauses 1 to 41 wherein the administration is in a parenteral dosage form.

43. The method of clause 42 wherein the parenteral dosage form is selected from an intradermal dosage form, a subcutaneous dosage form, an intramuscular dosage form, an intraperitoneal dosage form, an intravenous dosage form, and an intrathecal dosage form.

44. The method of any one of clauses 1 to 43 wherein the therapeutically effective amount or the diagnostically effective amount is from about 0.5 mg/m² to about 6.0 mg/m².

45. The method of any one of clauses 1 to 44 wherein the therapeutically effective amount or the diagnostically effective amount is from about 0.5 mg/m² to about 4.0 mg/m².

46. The method of any one of clauses 1 to 45 wherein the therapeutically effective amount or the diagnostically effective amount is from about 0.5 mg/m² to about 2.0 mg/m².

47. The method of any one of clauses 1 to 7 or 9 to 46 wherein the cancer is folate receptor-negative and the cancer is selected from colon cancer, lung cancer, prostate cancer, and breast cancer.

In one embodiment, targeting of MDSCs to deplete or to inhibit the activity of MDSCs can result in inhibition of tumor growth, complete or partial elimination of a tumor, stable disease, killing of tumor cells, and like therapeutic effects for the host animal. As used herein, to “deplete” or “inhibit” MDSCs means to kill some or all of a population of MDSCs, to inhibit or eliminate the activity of MDSCs (e.g., reducing or eliminating the ability of MDSCs to stimulate angiogenesis in tumor tissue), to reprogram MDSCs so that MDSCs inhibit rather than support tumor survival, to prevent an increase in numbers of MDSCs or reduce the number of MDSCs, or to have any other effect on MDSCs that results in an anti-cancer therapeutic effect for the host animal.

The methods described herein are used to treat a “host animal” with cancer in need of such treatment. In one embodiment, the methods described herein can be used for human clinical medicine or veterinary applications. Thus, a “host animal” can be administered the one or more compound(s) or a folate-imaging agent conjugate as described herein (described below), and the host animal can be human (e.g. a human patient) or, in the case of veterinary applications, can be a laboratory, agricultural, domestic, or wild animal. In one aspect, the host animal can be a human, a laboratory animal such as a rodent (e.g., mice, rats, hamsters, etc.), a rabbit, a monkey, a chimpanzee, domestic animals such as dogs, cats, and rabbits, agricultural animals such as cows, horses, pigs, sheep, goats, and wild animals in captivity such as hears, pandas, lions, tigers, leopards, elephants, zebras, giraffes, gorillas, dolphins, and whales.

In various embodiments, the cancers described herein can be cancers that are tumorigenic, including benign tumors and malignant tumors, or the cancer can be non-tumorigenic. In one embodiment, the cancer can arise spontaneously or by such processes as mutations present in the germline of the host animal or by somatic mutations, or the cancer can be chemically-, virally-, or radiation-induced. In another embodiment, cancers applicable to the invention described herein include, but are not limited to, a carcinoma, a sarcoma, a lymphoma, a melanoma, a mesothelioma, a nasopharyngeal carcinoma, a leukemia, an adenocarcinoma, and a myeloma.

In some aspects, the cancer can be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, cancer of the urethra, prostate cancer, thymoma, thymus cancer, leukemia, lymphoma, pleural mesothelioma, cancer of the bladder, Burkitt's lymphoma, cancer of the ureter, cancer of the kidney, neoplasms of the central nervous system, brain cancer, pituitary adenoma, or adenocarcinoma of the gastroesophageal junction.

In some aspects, the cancer can be selected from the group consisting of non-small cell lung cancer, anaplastic thyroid cancer, pancreatic ductal adenocarcinoma, head and neck cancer, epidermal growth factor receptor negative breast cancer, mesothelioma, adult classical Hodgkin's lymphoma, uveal melanoma, glioblastoma, renal carcinoma, leiomyosarcoma, and pigmented villonodular synovitis.

In another embodiment, the cancer is selected from non-small cell lung cancer, head and neck cancer, triple negative breast cancer, breast cancer, ovarian cancer, colon cancer, prostate cancer, lung cancer, endometrial cancer, and renal cancer.

In another embodiment, the cancer is folate receptor-negative and the cancer is selected from colon cancer, lung cancer, prostate cancer, and breast cancer. Any cancer that has MDSCs associated with it can be treated in accordance with the methods described herein.

Illustrative embodiments of “a folate,” that is part of a folate receptor binding ligand, include folic acid, and analogs and derivatives of folic acid, such as folinic acid, pteroylpolyglutamic acid, pteroyl-D-glutamic acid, and folate receptor-binding pteridines such as tetrahydropterins, dihydrofolates, tetrahydrofolates, and their deaza and dideaza analogs. The terms “deaza” and “dideaza” analogs refer to the art-recognized analogs having a carbon atom substituted for one or two nitrogen atoms in the naturally occurring folic acid structure, or analog or derivative thereof. For example, the deaza analogs include the 1-deaza, 3-deaza, 5-deaza, 8-deaza, and 10-deaza analogs of folate, folinic acid, pteropolyglutamic acid, and folate receptor-binding pteridines such as tetrahydropterins, dihydrofolates, and tetrahydrofolates. The dideaza analogs include, for example, 1,5-dideaza, 5,10-dideaza, 8,10-dideaza, and 5,8-dideaza analogs of folate, folinic acid, pteropolyglutamic acid, and folate receptor-binding pteridines such as tetrahydropterins, dihydrofolates, and tetrahydrofolates. Other folates useful as complex forming ligands for this invention are the folate receptor-binding analogs aminopterin, amethopterin (also known as methotrexate), N¹⁰-methylfolate, 2-deamino-hydroxyfolate, deaza analogs such as 1-deazamethopterin or 3-deazamethopterin, and 3′,5′-dichloro-4-amino-4-deoxy-N¹⁰-methylpteroylglutamic acid (dichloromethotrexate). Additional folates (for example, analogs of folic acid) that bind to folate receptors are described in U.S. Patent Application Publication Nos. 2005/0227985 and 2004/0242582, the disclosures of which are incorporated herein by reference. Folic acid, and the foregoing analogs and/or derivatives are also termed “a folate,” “the folate,” or “folates” reflecting their ability to bind to folate-receptors, and such ligands when conjugated with exogenous molecules are effective to enhance transmembrane transport, such as via folate-mediated endocytosis. The foregoing can be used in the folate receptor binding ligands described herein.

In one embodiment the folate receptor binding ligands described herein can be linked to a drug via a linker to make the compounds for use in the methods described herein. Any drug suitable for depleting or inhibiting MDSCs can be used in accordance with the methods described herein. In one embodiment, the drug is selected from CI307, vinblastine, GDC0980, BEZ235, wortmannin, AMT, PF-04691502, a CpG oligonucleotide, BLZ945, lenalidomide, NLG919, 5,15-DPP, a pyrrolobenzodiazepine, methotrexate, everolimus, tubulysin, GDC-0980, AS1517499, BIRB796, n-acetyl-5-hydroxytryptamine, and 2,4-diamino-6-hydroxpyrimidine.

In one aspect, the drug can be a microtubule inhibitor. In this embodiment, the drug can kill myeloid-derived suppressor cells, and the drug can be a tubulysin.

In another embodiment, the drug is selected from a PI3K inhibitor, a STAT6 inhibitor, a MAPK inhibitor, an iNOS inhibitor, and an anti-inflammatory drug. In this embodiment, the drug can inactivate myeloid-derived suppressor cells. In this embodiment, the drug can be a PI3K inhibitor, selected from GDC-0980, wortmannin, and PF-04691502, a STAT6 inhibitor (e.g., AS1517499), a MAPK inhibitor (e.g., BIRB796), an iNOS inhibitor (e.g., AMT), or an anti-inflammatory drug (e.g., methotrexate).

In yet another embodiment, the drug can be a TLR agonist, such as a TLR7 agonist, a TLR9 agonist, a TLR3 agonist (e.g., Poly: IC), or a TLR7/8 agonist (e.g., imiquimod). The TLR agonist can be selected, for example, from CI307, a CpG oligonucleotide, and TLR7A. In this embodiment, the drug can reprogram myeloid-derived suppressor cells.

In still another embodiment, the drug can be selected from the group consisting of a DNA-alkylating agent or DNA-intercalating agent (e.g. a PBD, pro-PBD or Hoechst stain), trabectedin, doxorubicin, gemcitabine, a bisphosphonate (e.g., free or in liposomal form), and a proapoptotic peptide. In yet another embodiment, the drug can be selected from the group consisting of monophosphoryl lipid A (e.g., detoxified LPS), an mTOR inhibitor (e.g., an everolimus or a rapamycin), a PPARγ agonist, and a PPARδ agonist.

In another aspect, the drug can be selected from the group consisting of silibinin, a src kinase inhibitor, a MerTK inhibitor, and a Stat3 inhibitor. In this embodiment, the drug can be a src kinase inhibitor (e.g., dasatinib). In another embodiment, the drug can be a MerTK inhibitor (e.g., UNC1062). In yet another embodiment, the drug can be a Stat3 inhibitor (e.g., selected from sunitinib and sorafenib).

It is to be understood that analogs or derivatives of the drugs described herein may also be used in the compounds described herein. The drug can also be an imaging agent linked to a folate receptor binding ligand via a linker.

In another aspect, more than one compound can be administered and the compounds can comprise different drugs. In one embodiment, the different drugs can be selected from, for example, a TLR7 agonist and a PI3K inhibitor. In yet another embodiment, one or more compounds can be administered along with one or more unconjugated drugs (i.e., not linked to a folate receptor binding ligand). For the combination therapy embodiments, any of the compounds and drugs described herein may be used, or other drugs that deplete or inhibit MDSCs can be used in accordance with the methods described herein. For the combination therapy embodiments, synergism may result as is described herein.

In one embodiment, before a host animal is treated with the methods described herein to deplete or inhibit MDSCs, the host animal can be treated by administering a folate-imaging agent conjugate to the host animal to determine the host animal's folate receptor status, as described in U.S. Appl. Publ. No. 20140140925, incorporated herein by reference. In this embodiment, the host animal's folate receptor status can be determined to be positive or negative, and the folate receptor status can be used to determine the compound that should be administered to the host animal.

In a further aspect of the methods described herein, the folate in the one or more compounds is selected from a folate specific for the folate receptor-α and a folate specific for the folate receptor-f3. In this aspect, at least two compounds can be administered and the folate in one compound is a folate specific for the folate receptor-α and the folate in the other compound is specific for the folate receptor-β. In this illustrative aspect, folate receptor positive cancers can be treated by treating the tumor directly through binding of the compound to the tumor and treating the tumor indirectly by binding of another compound to MDSCs to inhibit or deplete MDSCs.

In another embodiment, the compound has the formula

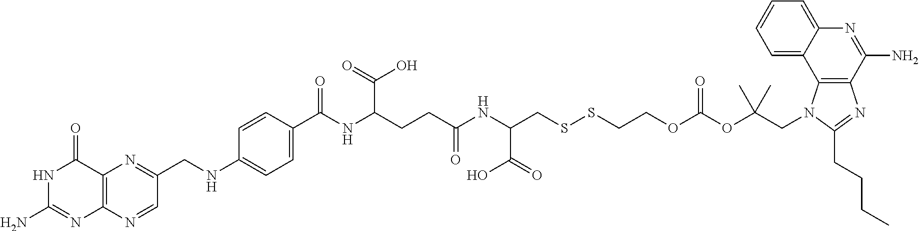 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

(also referred to herein as FA-TLR7), or a pharmaceutically acceptable salt thereof.

In another embodiment, the compound has the formula

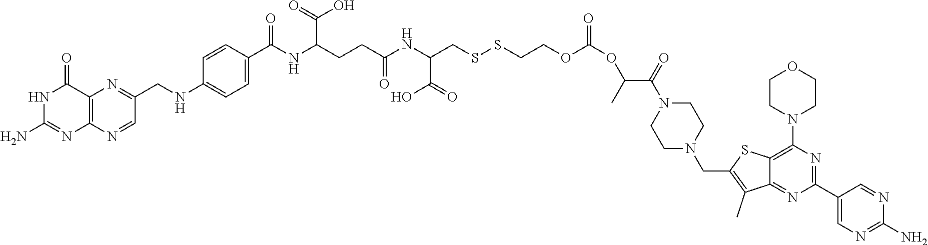 [SMILES: Cc1c(CN2CCN(C(=O)C(C)OC(=O)OCCSSCC(NC(=O)CCC(NC(=O)c3ccc(NCc4cnc5nc(N)[nH]c(=O)c5n4)cc3)C(=O)O)C(=O)O)CC2)sc2c(N3CCOCC3)nc(-c3cnc(N)nc3)nc12]

(also referred to herein as FA-PI3K) or a pharmaceutically acceptable salt thereof.

In another embodiment, the compound has the formula

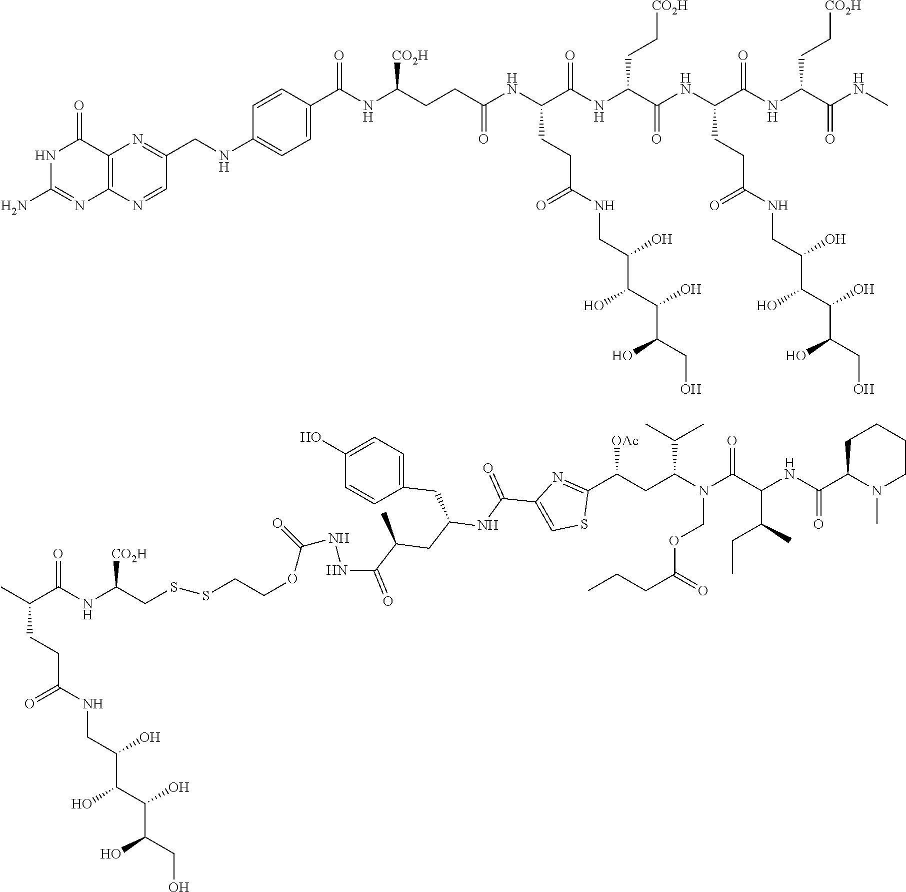

(also referred to herein as FA-tubulysin) or a pharmaceutically acceptable salt thereof.

In another embodiment, the compound has the formula

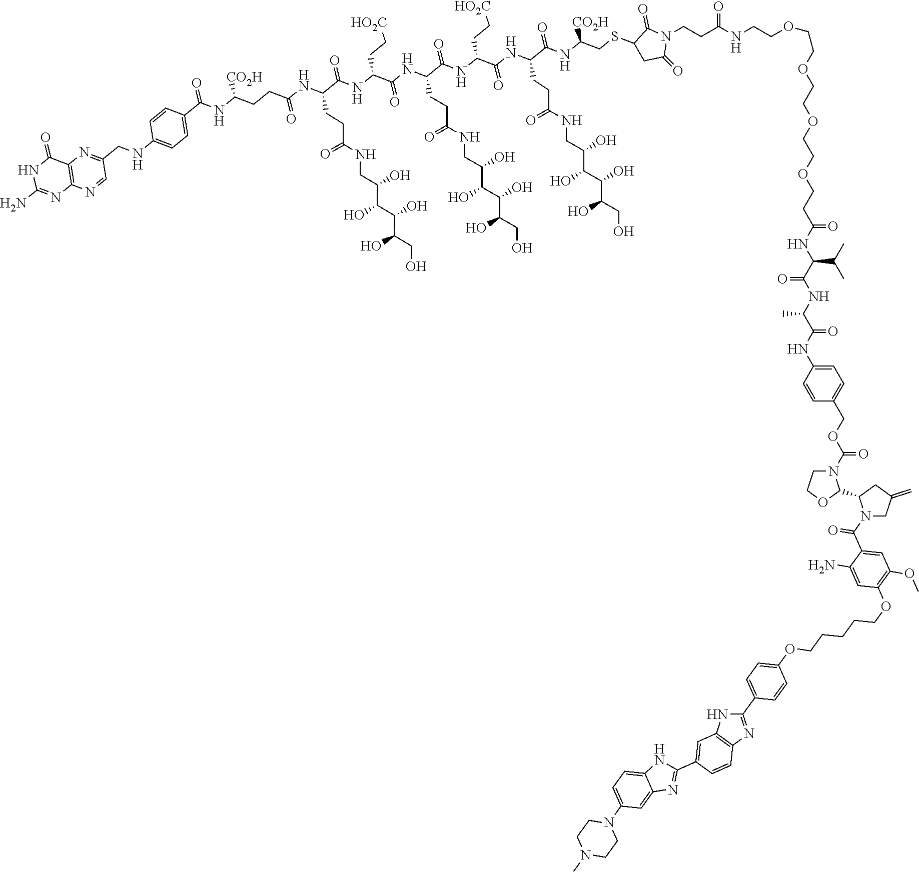 [SMILES: C=C1C[C@@H](C2OCCN2C(=O)OCc2ccc(NC(=O)[C@H](C)NC(=O)[C@@H](NC(=O)CCOCCOCCOCCOCCNC(=O)CCN3C(=O)CC(SC[C@@H](NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)[C@@H](CCC(=O)O)NC(=O)[C@H](CCC(=O)NC[C@H](O)[C@@H](O)[C@H](O)[C@H](O)CO)NC(=O)CC[C@H](NC(=O)c4ccc(NCc5cnc6nc(N)[nH]c(=O)c6n5)cc4)C(=O)O)C(=O)O)C3=O)C(C)C)cc2)N(C(=O)c2cc(OC)c(OCCCCCOc3ccc(-c4nc5ccc(-c6nc7cc(N8CCN(C)CC8)ccc7[nH]6)cc5[nH]4)cc3)cc2N)C1]

(also referred to herein as FA-PBD) or a pharmaceutically acceptable salt thereof.

As used herein, the term “pharmaceutically acceptable salt” refers to those salts with counter ions which may be used in pharmaceuticals. Such salts include (1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well known to those skilled in the art, and any such pharmaceutically acceptable salts may be contemplated in connection with the embodiments described herein.

Suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts of the compounds described herein are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one aspect, a compound as described herein may be administered directly into the blood stream, into muscle, or into an internal organ. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. Suitable means for parenteral administration include needle (including microneedle) injectors, needle-free injectors, and infusion techniques.

In one illustrative aspect, parenteral compositions are typically aqueous solutions which may contain carriers or excipients such as salts, carbohydrates and buffering agents (preferably at a pH of from 3 to 9), but, for some applications, they may be more suitably formulated as a sterile non-aqueous solution or as a dried form to be used in conjunction with a suitable vehicle such as sterile, pyrogen-free water or phosphate-buffered saline. In other embodiments, any of the compositions containing the compounds described herein may be adapted for parenteral administration of the compounds as described herein. The preparation of parenteral compositions under sterile conditions, for example, by lyophilization under sterile conditions, may readily be accomplished using standard pharmaceutical techniques well known to those skilled in the art. In one embodiment, the solubility of a compound used in the preparation of a parenteral composition may be increased by the use of appropriate formulation techniques, such as the incorporation of solubility-enhancing agents.

The dosage of the compound can vary significantly depending on the condition of the host animal, the cancer being treated, the route of administration of the compound and tissue distribution, and the possibility of co-usage of other therapeutic treatments, such as radiation therapy or additional drugs in combination therapies. The therapeutically effective amount (i.e., compounds) or diagnostically effective amount (e.g., folate-imaging agent conjugates as described in U.S. Appl. Publ. No. 20140140925, incorporated herein by reference) to be administered to a host animal is based on body surface area, mass, and physician assessment of condition of the host animal. Therapeutically effective or diagnostically effective amounts can range, for example, from about 0.05 mg/kg of patient body weight to about 30.0 mg/kg of patient body weight, or from about 0.01 mg/kg of patient body weight to about 5.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kg of patient body weight. The total therapeutically or diagnostically effective amount of the compound may be administered in single or divided doses and may, at the physician's discretion, fall outside of the typical range given herein.

In another embodiment, the compound or the folate-imaging agent conjugate can be administered in a therapeutically or diagnostically effective amount of from about 0.5 μg/m² to about 500 mg/m², from about 0.5 μg/m² to about 300 mg/m², or from about 100 μg/m² to about 200 mg/m². In other embodiments, the amounts can be from about 0.5 mg/m² to about 500 mg/m², from about 0.5 mg/m² to about 300 mg/m², from about 0.5 mg/m² to about 200 mg/m², from about 0.5 mg/m² to about 100 mg/m², from about 0.5 mg/m²′ to about 50 mg/m², from about 0.5 mg/m² to about 600 mg/m², from about 0.5 mg/m² to about 6.0 mg/m², from about 0.5 mg/m² to about 4.0 mg/m², or from about 0.5 mg/m² to about 2.0 mg/m². The total amount may be administered in single or divided doses and may, at the physician's discretion, fall outside of the typical range given herein. These amounts are based on m² of body surface area.

The compounds described herein may contain one or more chiral centers, or may otherwise be capable of existing as multiple stereoisomers. It is to be understood that in one embodiment, the invention described herein is not limited to any particular stereochemical requirement, and that the compounds may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configurations at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds may be pure, or may be any of a variety of geometric isomer mixtures. It is also to be understood that such mixtures of geometric isomers may include a single configuration at one or more double bonds, while including mixtures of geometry at one or more other double bonds.

As used herein, the term “linker” includes a chain of atoms that connects two or more functional parts of a molecule to form a compound of the invention. Illustratively, the chain of atoms is selected from C, N, O, S, Si, and P, or C, N, O, S, and P, C, N, O, and S. The chain of atoms covalently connects different functional capabilities of the compound, such as the folate and the drug. The linker may have a wide variety of lengths, such as in the range from about 2 to about 100 atoms in the contiguous backbone.

As used herein, the term “releasable linker” or “linker that is releasable” refers to a linker that includes at least one bond that can be broken under physiological conditions, such as a pH-labile, acid-labile, base-labile, oxidatively-labile, metabolically-labile, biochemically-labile, or enzyme-labile bond. It is appreciated that such physiological conditions resulting in bond breaking do not necessarily include a biological or metabolic process, and instead may include a standard chemical reaction, such as a hydrolysis reaction, for example, at physiological pH, or as a result of compartmentalization into a cellular organelle such as an endosome having a lower pH than cytosolic pH.

It is understood that a cleavable bond can connect two adjacent atoms within the releasable linker and/or connect other linker portions or the folate and/or the drug, as described herein, at either or both ends of the releasable linker. In the case where a cleavable bond connects two adjacent atoms within the releasable linker, following breakage of the bond, the releasable linker is broken into two or more fragments. Alternatively, in the case where a cleavable bond is between the releasable linker and another moiety, following breakage of the bond, the releasable linker is separated from the other moiety.

In another embodiment, compositions for administration of the compound are prepared from the compound with a purity of at least about 90%, or about 95%, or about 96%, or about 97%, or about 98%, or about 99%, or about 99.5%. In another embodiment, compositions for administration of the compound are prepared from the compound with a purity of at least 90%, or at least 95%, or at least 96%, or at least 97%, or at least 98%, or at least 99%, or at least 99.5%.

### EXAMPLES

### Chemicals and Reagents:

Fmoc-Glu-OtBu was purchased from AAPPTEC Inc. 4-Chloro-3-nitroquinoline was purchased from Matrix Scientific Inc. Fmoc-8-amino-3,6-dioxaoctanoic acid was purchased from PolyPeptide Inc. N10-(trifluoroacetyl)pteroic acid, tubulysin were provided by Endocyte Inc. Solid phase synthesis monitor kit was purchased from ANASPEC Inc. 2,2-dimethyloxirane, ammonium hydroxide, di-tert-butyl dicarbonate, trifluoroacetic acid, toluene, 2-propanol, methanol, Pd/C, 1,2-diaminoethane trityl (polymer-bound resin), triethylamine, valeryl chloride, ethyl acetate, hexane, Na₂SO₄, calcium oxide, dichloromethane, 3-chloroperoxybenzoic acid, benzoyl isocyanate, H-cys(Trt)-2-chlorotrityl resin, sodium methoxide, dimethylaminopyridine, acetonitrile, DMSO, 4-chloro-3-nitro-a,a,a-trifluorotoluene, hydrazine hydrate, ethanol, Na₂CO₃, NaHCO₃, concentrated HCl, ether, trichloromethylchloroformate, sulfuryl chloride, 2-mercapropyridine, 2-mercaptoethanol, DMF, PyBOP, DIPEA, ethanedithiol, thiisoproylsilane, 20% piperidine DMF solution, 4-chloro-3-nitro-a,a,a-trifluorotoluene, hydrazine hydrate, 5,15-DPP, resiquimod, 2,4-diamino-6-hydroxypyrimidinc, N-acetyl-5-hydroxytryptamine, methotrexate, everolimus, zymosan, MnCl₂, L-arginine, dulbecco's phosphate buffered saline (PBS), collagenase from Clostridium histolyticum, deoxyribonuclease I from bovine pancreas, hyaluronidase from bovine testes, bovine scrum albumin (BSA), glycine, sodium azide, OPD substrate were purchased from Sigma. Compressed gases of hydrogen, argon, nitrogen were purchased from Indiana Oxygen Company. BEZ235, PF-04691502, GDC-0980, wortmannin, BLZ945, lenalidomide, NLG 919, AS1517499, and BIRB796 were purchased from Selleckchem. AMT was purchased from Tocris Bioscience. CL307, CpG, and Poly IC were purchased from InvivoGen Inc. Greiss reagent was purchased from Lifetechnology Inc. 10% Triton X-100 was purchased from Pierce Inc. Protease inhibitor was purchased from Research Products International. QuantiChrom™ urea assay kit was purchased from BioAssay Systems. Mouse IL-10 duoset, and anti-mouse FITC-arginase were purchased from R&D systems. RPMI 1640 medium, folate-deficient RPMI 1640 medium were purchased from Gibco Inc. Penicillin streptomycin solution (50×), L-glutamine (200 mM), 0.25% trypsin with 2.21 mM EDTA (1×) were purchased from Corning Inc. Fetal bovine serum (FBS) was purchased from Atlanta biologicals Inc. Folate deficient diet for animals was purchased from Envigo Inc. Mouse folate receptor-f3 antibody (F3IgG2a) was provided by Dr. Dimitrov from NIH. Mouse Fc blocker (CD16/CD32), anti-mouse FITC-CD11b, anti-mouse PE-F4/80, anti-mouse PE-Gr1, anti-mouse PE-CD4, anti-mouse FITC-CD8, 7-AAD viability staining solution, red blood cell lysis buffer (10×) were purchased from Biolegend Inc. Fixable viability dye eFluor® 660 was purchased from eBioscience, Inc. Pierce™ 16% formaldehyde (w/v) (methanol-free) was purchased from Thermo Fischer Scientific. Isoflurane was purchased from VetOne Inc. Andy Fluor™ 647 NHS ester (succinimidyl ester) was purchased from Applied Bioprobes. Mouse GM-CSF was purchased from Miltenyi Biotec Inc. Folate-tubulysin was prepared according to literature procedure (see for example the procedure describe in WO2014/062697). Anti human APC-CD33 antibody was purchased from Biolegend Inc. Human T cell culture media (TexMACS medium), Human IL-2 were purchased from Miltenyi Biotech. Human T cell isolation kit (Human T cell Enrichment Kit) was purchased from STEMMCELL. Ficoll-Paque™ Plus was purchased from GE Healthcare. 6-thioguanine and methylene blue were purchased from Sigma.

### BIOLOGY EXAMPLES

### Example 1: Cell Culture and Animal Husbandry

4T1 cells which do not express folate receptor were provided by Endocyte Inc. Cells were cultured in completed RPMI 1640 medium (RPMI 1640 medium supplemented with 10% fetal bovine serum, 1% penicillin streptomycin and 2 mM L-Glutamine) at 37° C. in a humidified 95% air 5% CO/atmosphere. Cell medium was spiked with 0.25% trypsin with 2.21 mM EDTA every 3 to 4 days. Female balb/c mice at 6 to 8 weeks of age were obtained from Envigo Inc. Animals were maintained on normal rodent chow or folate deficient diet and housed in a sterile environment on a standard 12 h light and dark cycle for the duration of the study. All animal procedures were approved by the Purdue Animal Care and Use Committee in accordance with NIH guidelines.

### Example 2: Tumor Models

4T1 solid tumor model: Female balb/c mice at the age of 6 to 8 weeks were kept on a folate deficient diet for 2 weeks. Before tumor implantation, fur on the left side of the mouse body was removed by an electric trimmer. 0.05 million 4T1 cells suspended in 50 μL completed RPMI 1640 medium were subcutaneously implanted near the mammary fat pad. Treatment was commenced at day 6 when the volume of tumor reached around 20 to 50 mm³. For characterization of FR⁺ TAMs/MDSCs, tumors were digested when the volume reached 300 to 500 mm³. Tumor digestion was developed which caused the least damage to cell surface proteins. The digestion cocktail was composed of 1 mg/mL collagenase IV, 0.1 mg/mL hyaluronidase from bovine tests, and 0.2 mg/mL deoxyribonuclease I in 10 mL serum free folate-deficient RPMI 1640 medium. Following digestion for 1 hour at 37° C. with mild shaking, the digestion reaction was stopped by addition of folate-deficient RPMI 1640 medium containing 10% heat inactivated FBS and the broken down tumors were passed through a 40 μm cell strainer to collect individual cells. The isolated cells were then spun down to remove digestion cocktail and re-suspended in 5 to 10 mL red blood cell lysis buffer (1×) for 5 min on ice. 30 to 40 mL of PBS was added to stop the cell lysis reaction. Cells were then spun down to remove the supernatant and re-suspended in flow staining medium, which was PBS containing 2% FBS. Cells were counted and were then ready for flow cytometry staining.

4T1 peritoneal model: Female balb/c mice at the age of 6 to 8 weeks were kept on normal rodent chow. 10 million 4T1 cells in 300 μL PBS were injected into the peritoneal cavity. Peritoneal ascites were collected between day 7 and day 10 by peritoneal lavage. Cells were spun down to remove the supernatant and re-suspended in 5 to 10 mL red blood cell lysis buffer (1×) for 5 min on ice. 30 to 40 mL of PBS was added to stop the cell lysis reaction. Cells were then spun down to remove the supernatant and re-suspended in completed RPMI 1640 medium supplemented with 10 ng/mL mouse GM-CSF. Cells were counted and ready for flow cytometry staining and in vitro screening.

RM1 solid tumor model: Male C57BL/6 mice at the age of 6 to 8 weeks were kept on a folate deficient diet for 2 weeks. Before tumor implantation, fur on the mouse neck was removed by an electric trimmer. 2 million RM1 cells suspended in 50 μL completed RPMI 1640 medium were subcutaneously implanted. The animals were monitored every other day after tumor implantation. When the tumor size reached around 500 mm³, mice were euthanized. The tumor was digested using a cocktail similar to the 4T1 tumor model. Following digestion for 1 hour at 37° C. with mild shaking, the digestion reaction was stopped by addition of folate-deficient RPMI 1640 medium containing 10% heat inactivated FBS and the broken down tumors were passed through a 40 μm cell strainer to collect individual cells. The isolated cells were then spun down to remove digestion cocktail and re-suspended in 5 to 10 mL red blood cell lysis buffer (1×) for 5 min on ice. 30 to 40 mL of PBS was added to stop the cell lysis reaction. Cells were then spun down to remove the supernatant and re-suspended in flow staining medium, which was PBS containing 2% FBS. Cells were counted and were then ready for flow cytometry staining.

CT26 solid tumor model: Female Balb/C mice at the age of 6 to 8 weeks were kept on a folate deficient diet for 2 weeks. Before tumor implantation, fur on the mouse neck was removed by an electric trimmer. 2 million CT26 cells suspended in 50 μL completed RPMI 1640 medium were subcutaneously implanted. The animals were monitored every other day after tumor implantation. When the tumor size reached around 500 mm³, mice were euthanized. The tumor was digested using the similar cocktail as in 4T1 tumor model. Following digestion for 1 hour at 37° C. with mild shaking, the digestion reaction was stopped by addition of folate-deficient RPMI 1640 medium containing 10% heat inactivated FBS and the broken down tumors were passed through a 40 μm cell strainer to collect individual cells. The isolated cells were then spun down to remove digestion cocktail and re-suspended in 5 to 10 mL red blood cell lysis buffer (1×) for 5 min on ice. 30 to 40 mL of PBS was added to stop cell lysis reaction. Cells were then spun down to remove supernatant and re-suspended in flow staining medium, which was PBS containing 2% FBS. Cells were counted and were then ready for flow cytometry staining.

EMT6 solid tumor model: Female Balb/C mice at the age of 6 to 8 weeks were kept on folate deficient diet for 2 weeks. Before tumor implantation, fur on the mouse neck was removed by an electric trimmer. 2 million EMT6 cells suspended in 50 μL completed RPMI 1640 medium were subcutaneously implanted. The animals were monitored every other day after tumor implantation. When the tumor size reached around 500 mm³, mice were euthanized. The tumor was digested using the similar cocktail as in 4T1 tumor model. Following digestion for 1 hour at 37° C. with mild shaking, the digestion reaction was stopped by addition of folate-deficient RPMI 1640 medium containing 10% heat inactivated FBS and the broken down tumors were passed through a 40 μm cell strainer to collect individual cells. The isolated cells were then spun down to remove digestion cocktail and re-suspended in 5 to 10 mL red blood cell lysis buffer (1×) for 5 min on ice. 30 to 40 mL of PBS was added to stop the cell lysis reaction. Cells were then spun down to remove supernatant and re-suspended in flow staining medium, which was PBS containing 2% FBS. Cells were counted and were then ready for flow cytometry staining.

### Example 3: Flow Cytometry Analysis

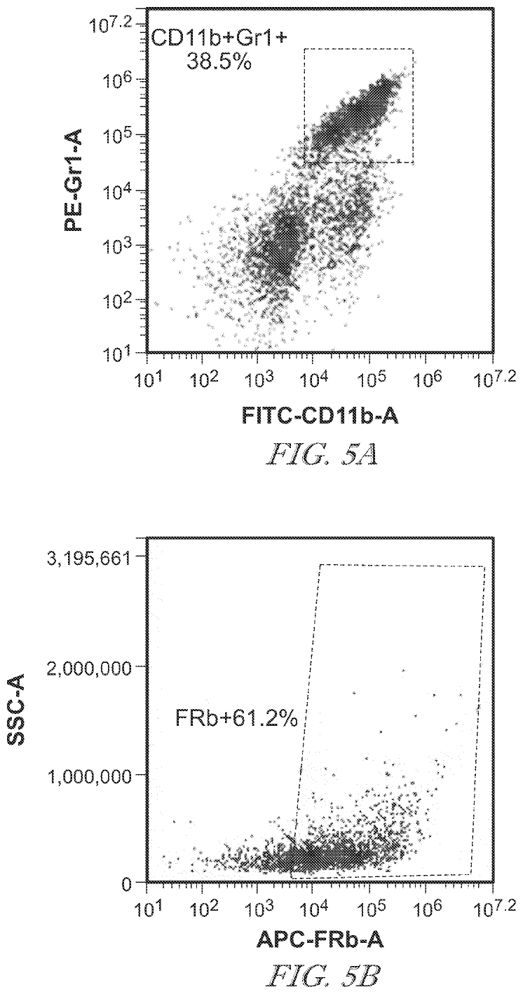

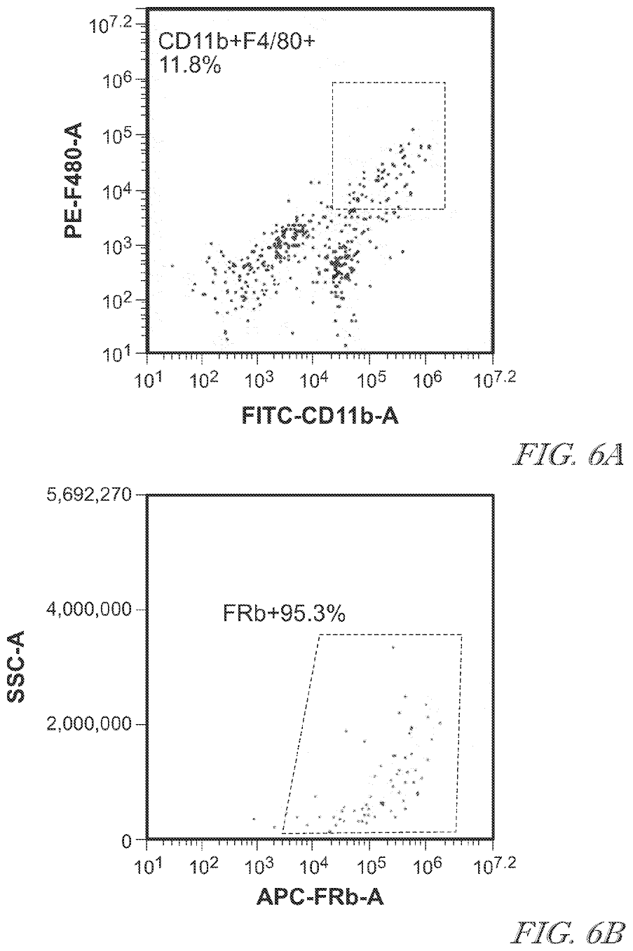

Cell surface marker staining: Single-cell suspensions obtained from the solid tumor model or peritoneal tumor model were prepared as previous mentioned. One million cells in 100 μL flow staining medium were incubated with 0.7 μL mouse Fc blocker for 5 min on ice. Surface markers for MDSCs (CD11b, Gr1), TAMs (CD11b, F4/80), and folate receptor-β (F3IgG2a) were added after incubation with Fc blocker. Table 1 and 2 listed volumes of antibodies used for surface marker staining. After incubation on ice for 1 hour, cells were washed with 500 μL PBS and re-suspended in 200 μL flow staining medium. Dead/live cell marker (3 μl of 7-AAD or 1 μl of BV421 dead/live) was added to each sample and incubated at room temperature in the dark. After 15 min, cells were analyzed using a BD Accuri C6TM flow cytometer without washing (Table 1 staining). One time washing was performed for Table 2 staining and cells were analyzed using a BD Forteassa flow cytometer. Results are shown in FIG. 5 and FIG. 6. As shown in FIG. 5 and FIG. 6, the mouse MDSCs and TAMs population in solid 4T1 tumor can be identified by CD11b+Gr1+ and CD11b+F4/80+ markers, respectively. After gated on these two populations of cells, FR-β expression could be observed on most of these two populations (61.2% on MDSCs and 95% on TAMs).

TABLE 1
Antibody volumes in 100 μL cell suspension for
flow cytometry staining of PDL-1 and FR-β.

| | | | | Alexa | | |
|---|---|---|---|---|---|---|
| | | | | Fluor | | |
| | BV605- | FITC- | PerCp/ | 647- | BV421 | AF594- |
| Antibody | Ly6C | CD11b | Cy5.5-Grl | F3IgG2a | dead/live | F4/80 |
| Volume | 0.5 μL | 1 μL | 0.5 μL | 0.5 μL | 1 μL | 0.5 μL |

Intracellular arginase staining: Cell surface markers for TAMs/MDSCs were labeled following procedures mentioned previously. 0.1 μL fixable viability dye eFluor® 660 was added together with antibody cocktails. After washing with PBS, cells were fixed with 4% formaldehyde in 500 μL of PBS for 15 min at 4° C. Cells were spun down to remove fixing solution. Cells were washed two times with 500 μL washing buffer containing 0.1 M glycine and 0.05% sodium azide. After being spun down a final time, cells were added 1 mL permeabilization solution containing 0.1 M glycine, 0.05% sodium azide and 0.1% triton-100. Permeabilization was performed at room temperature for 5 min. Permeabilized cells were spun down at 1500 rpm for 1 min, and the cells were washed three times with 1 mL blocking buffer containing 0.05 M glycine, 0.05% sodium azide and 0.2% gelatin. Cells were then re-suspended in 1 mL blocking buffer at 4° C. overnight to block non-specific intracellular binding. Cells were then spun down at 1500 rpm for 1 min to remove the supernatant and another 100 μL blocking buffer containing 1 μL FITC-arginase was added. Cells were kept in the dark at 4° C. overnight. After being spun down at 1500 rpm for 1 min, cells were washed with 1 mL blocking buffer and were then ready for flow cytometry analysis (BD Accuri C6™ flow cytometer).

### Example 4: In Vitro TAMs/MDSCs Screening

Cells isolated from the peritoneal model were re-suspended in completed RPMI 1640 medium supplemented with 10 ng/mL mouse GM-CSF and seeded into 96 well plates. Different concentrations of screening drugs listed in Table 2 were dissolved in the same medium and were added to each well containing 0.5 millions of cells in 300 μL medium. Wells containing 0.5 million cells in 300 μL medium without addition of drugs were kept as untreated control. Three extra wells were charged with 300 μL medium without cells and drugs to be kept as background control. Cells were then incubated at 37° C. in a humidified 95% air 5% CO₂ atmosphere for 24 hours to 48 hours. At the end of incubation, supernatant was collected for IL-10 ELISA and nitric oxide assay. Cells were washed twice with 300 μL PBS, and were then ready for the arginase assay.

TABLE 2
List of compounds and functions for in vitro screening

| Name | Function | Class | Name | Function | Class |
|---|---|---|---|---|---|
| CL307 | TLR7 | III | 5,15-DPP | STAT3 | II |
| | agonist | | | inhibitor | |
| | agonist | | | | |
| BEZ235 | PI3K | II | Methotrexate | Anti- | II |
| | inhibitor | | | inflammatory | |
| Wortmannin | PI3K | II | Everolimus | mTOR | II |
| | inhibitor | | | inhibitor | |
| AMT | iNOS | II | Tubulysin | microtubular | I |
| | inhibitor | | | inhibitor | |
| PF-04691502 | PI3K | II | GDC-0980 | PI3K inhibitor | II |
| | inhibitor | | | | |
| CpG | TLR9 | III | AS1517499 | STAT6 | II |
| | agonist | | | inhibitor | |
| BLZ945 | CSF-1R | II | BIRB796 | p38α MAPK | II |
| | inhibitor | | | inhibitor | |
| Lcnalidomide | TNF-α | II | N-Acety1-5- | BH4 inhibitor | II |
| | secretion | | hydroxy- | | |
| | inhibitor | | tryptamine | | |
| NLG 919 | IDO | II | 2,4-Diamino- | GTP | II |
| | pathway | | 6-hydroxypy- | cyclohydrolase | |
| | inhibitor | | rimidine | I inhibitor | |
| | | | (DAHP) | | |
| Poly I: C | TLR3 | III | vinblastine | microtubular | I |
| | agonist | | | inhibitor | |
| Zymosan | TLR5 | III | Am-9-79 | Topoisomerase | I |
| | agonist | | | I inhibitor | |

### Example 5: Arginase Assay

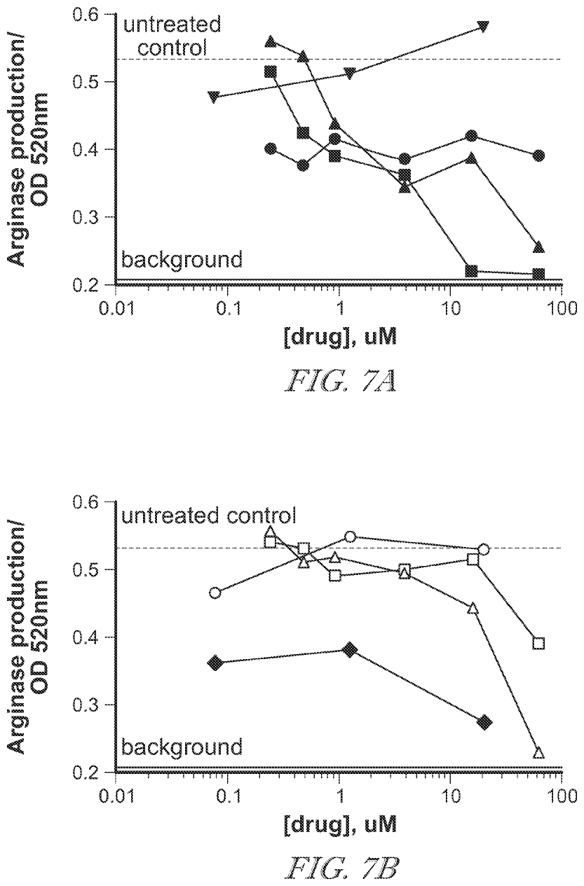

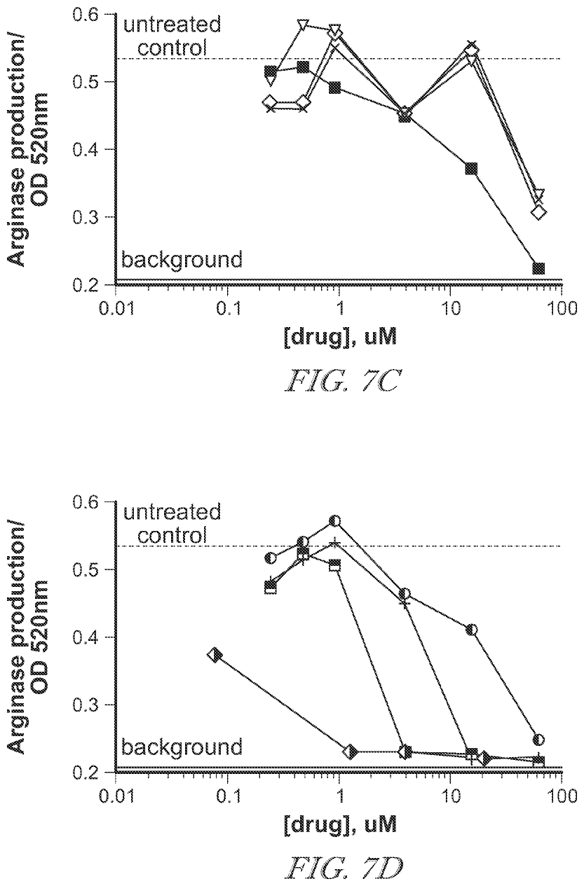

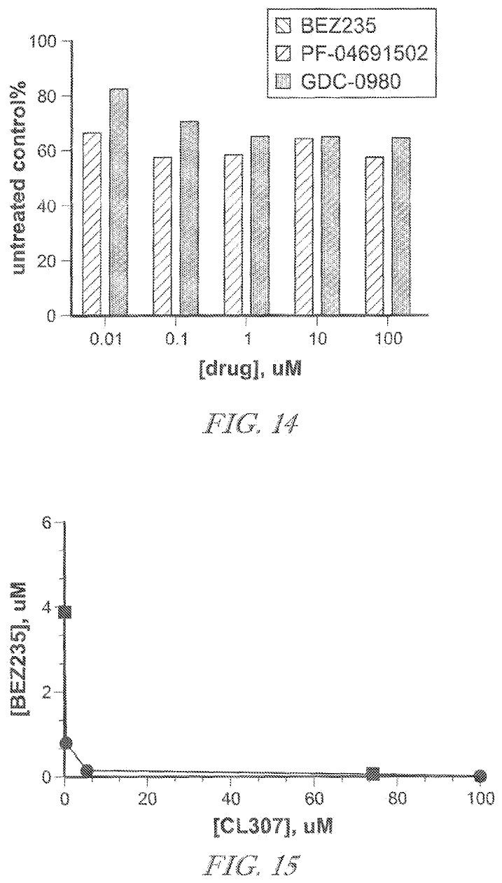

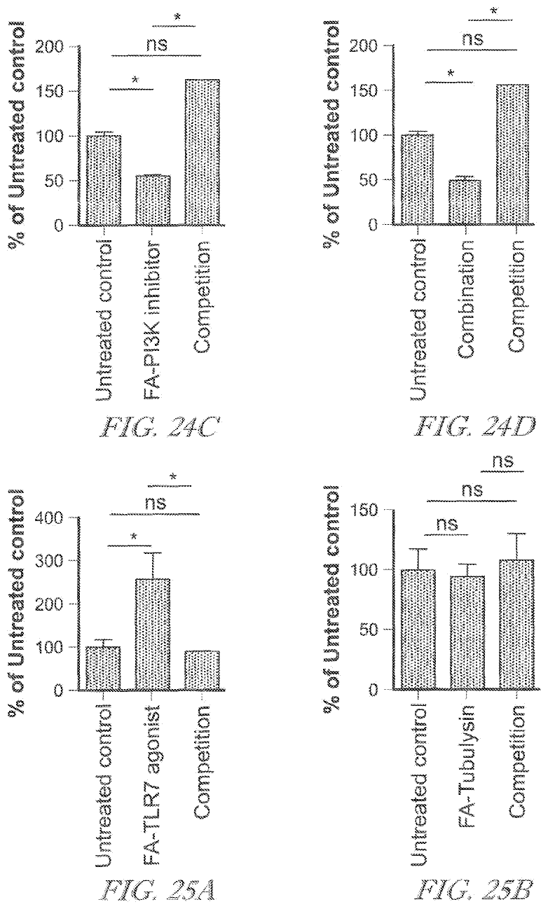

Arginase activity was measured in cell lysates as described in I. M. Corraliza, M. L. Campo, G. Soler, M. Modolell, ‘Determination of arginase activity in macrophages: a micromethod’, Journal of Immunological Methods 174 (1994) 231-235. Briefly, after in vitro incubation of isolated TAMs/MDSCs with different drugs in 96 well plates, cells were washed twice with 300 μL PBS. Cells were then lysed for 30 min at room temperature with 100 μL of 0.1% Triton X-100 with protease inhibitor (1×). Subsequently, 50 μL of the lysate solutions were transferred to a new V-shape 96 well plate. 50 μL of arginase activation solution (10 mM MnCl₂/50 mM Tris.Cl (pH 7.5)) were added into the cell lysate. The enzyme was activated by heating for 10 min at 56° C. Arginine hydrolysis was conducted by incubating 25 μL of the activated solution with 25 μL of arginase substrate solution (0.5 M L-arginine (pH 9.7)) at 37° C. for 60 min with mild shaking. After cooling to room temperature, 10 μL of the reaction solution was then diluted into 90 μL of PBS. 10 μL of this diluted solution was transferred to a 96 well flat bottom clear plate. 200 μL of urea reagents were added to each well. After incubation in the dark at room temperature for 10 min, the urea concentration was measured at 520 nm by plate reader. Results are shown in FIG. 7, FIG. 11, FIG. 12, FIG. 15 and FIG. 24.

As shown in FIG. 7, it was found that several drugs can efficiently decrease arginase production by TAMs/MDSCs in vitro, including CL307, BEZ235, wortmannin, CpG, tubulysin, AS1517499, and BIRB796. The concentration of arginase was proportional to the absorbance at 520 nm. The black dotted line in each Figure indicates arginase level of untreated control. The black solid line indicates arginase level of background. The absorbance at 520 nm for every sample was plotted vs concentrations of tested drugs from 0.1 μM to 100 μM.

As shown in FIG. 11, in order to test the potency of newly synthesized TLR7 agonist (TLR7A) on affecting arginase production by TAMs/MDSCs, TLR7A and C1307 were co-cultured with TAMs/MDSCs at different concentrations. From FIG. 11, it could be found that TLR7A is more efficient at decreasing arginase than a commercially available TLR7 agonist (C1307).

As shown in FIG. 12, by comparing the effect of three PI3K inhibitors at decreasing the production of arginase, by TAMs/MDSCs in vitro, it was found that GDC-0980 is the best candidate that can efficiently decrease arginase produced by TAMs/MDSCs.

As shown in FIG. 15, TAMs/MDSCs obtained from 4T1 peritoneal tumor model were cultured with TLR7 agonist (C1307), PI3K inhibitor (BEZ235) and/or a combination of two drugs at different concentrations. EC50 of every combination were plotted between two drugs as shown in FIG. 15. Square symbol indicated EC50 of single treatment with either C1307 or BEZ235. It was found that by combining two different drugs that can individually affect arginase production, a synergistic effect was observed, which can further decrease arginase production by TAMs/MDSCs.

As shown in FIG. 24, intracellular staining of arginase on F4/80+ macrophages was tested in groups of untreated control, FA-TLR7 agonist, FA-PI3K inhibitor, FA-Tubulysin and combination as well as competition groups. As described in the previous methods part, after tumor digestion at the end of the therapeutic studies, isolated cells were stained by macrophages surface marker (F4/80) and M2 macrophages functional marker (arginase) to test arginase expression level on F4/80+ macrophages. It has been established that arginase upregulation is an important suppression marker for TAMs/MDSCs since depletion of L-arginine by arginase can inhibit cytotoxic T cell proliferation. Arginase+F4/80+ cell population in live cells from treatment and competition groups were compared to the same population from untreated group. As shown in FIG. 24, arginase+F4/80+ cell population from treatment groups dramatically decreased compared with untreated control and this effect could be competed by extra addition of competitor (FA-PEG-NH₂). Therefore, it could be concluded that by targeting FR+ TAMs/MDSCs in 4T1 solid tumor, the three classes of FA-conjugated SMDCs are able to affect immunosuppression of TAMs/MDSCs.

### Example 6: IL-10 ELISA Assay

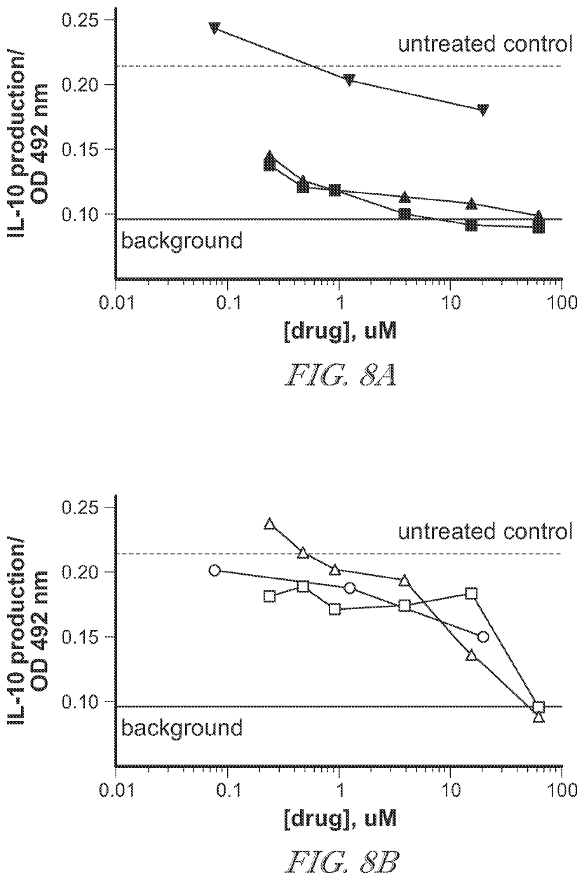

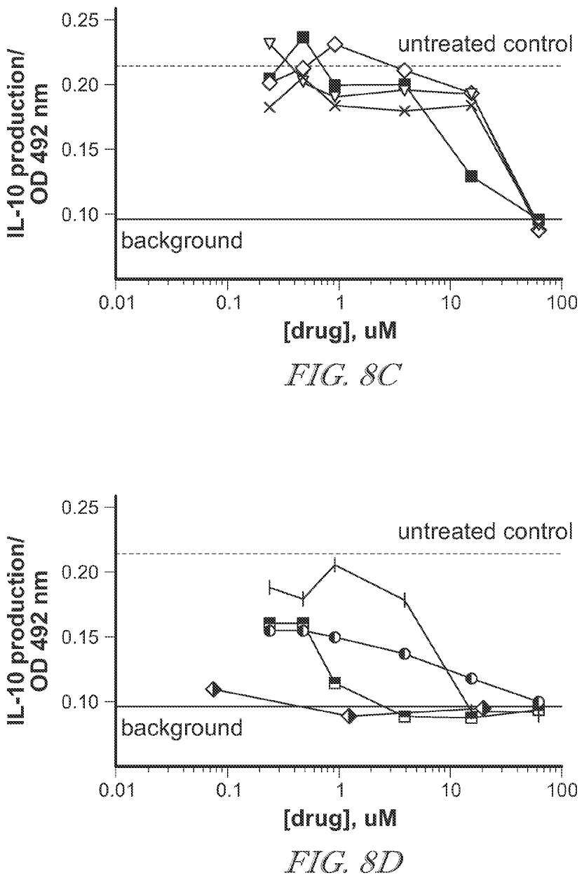

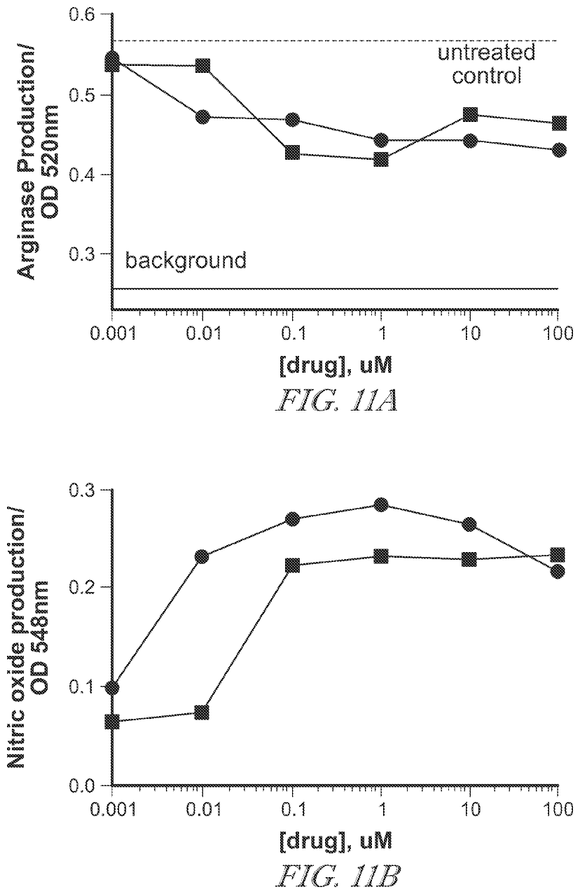

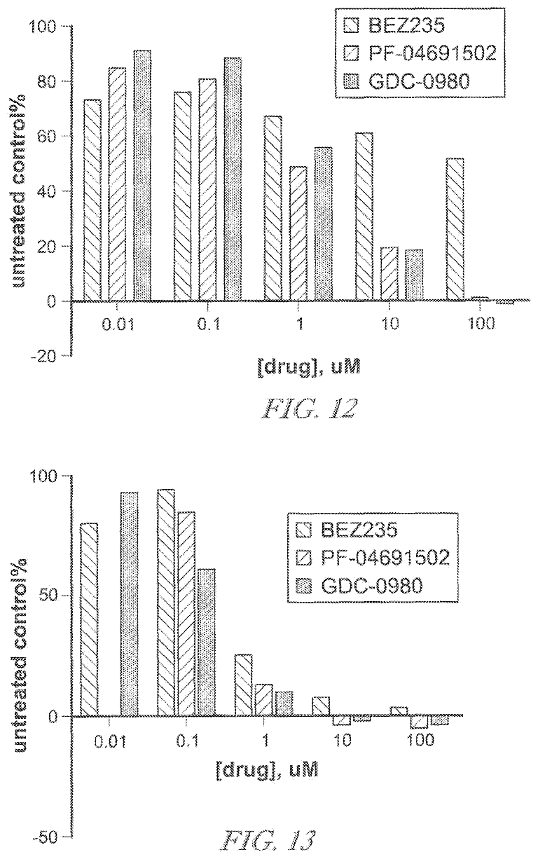

IL-10 production by TAMs/MDSCs after in vitro incubation with different compounds was determined by ELISA assay following the protocol provided with the Mouse IL-10 DuoSet ELISA by R&D Systems. Briefly, a high affinity 96-well plate was coated with 100 μL of diluted capture antibody per well with the working concentration of 4 μg/mL in PBS without carrier protein. The plate was sealed, and incubated overnight at room temperature. Each well was aspirated, and washed three times with 400 μL of wash buffer (0.05% Tween®20 in PBS, pH 7.2-7.4) using a squirt bottle. After the last wash, remaining wash buffer was removed by inverting the plate and blotting it against clean paper towels. The plates were blocked by adding 300 μL of reagent diluent (1% BSA in PBS, pH 7.2-7.4) to each well, and incubated at room temperature for 1 hour. Aspiration/wash was repeated three times in the same manner as previously described. The plates were ready for sample addition. 100 μL of sample supernatant from TAMs/MDSCs in vitro screening were added to each well. The plate was covered with an adhesive strip and incubated for 2 hours at room temperature. The previously mentioned aspiration/wash procedure was repeated three times. 100 μL of the detection antibody with the concentration of 300 ng/mL in reagent diluent was added to each well. The plate was covered with a new adhesive strip and incubated for 2 hours at room temperature. The previously mentioned aspiration/wash procedure was repeated three times. 100 μL of working dilution of streptavidin-HRP (1 to 40 dilution in reagent dilute) was added to each well. The plate was covered and incubated for 20 minutes at room temperature in dark. The previously mentioned aspiration/wash procedure was repeated three times. 200 μL of substrate solution (one bag of silver and golden tablets of OPD in 20 mL of DI water) was added to each well. The plate was incubated for 20 minutes at room temperature in dark. 50 μL of stop solution (3M HCl) was added to each well. The plate was gently tapped to ensure thorough mixing. The IL-10 concentration was proportional to the optical density determined by a microplate reader at 492 nm. Results are shown in FIG. 8 and FIG. 13.

As shown in FIG. 8, it was found that several drugs can efficiently decrease IL-10 production by TAMs/MDSCs in vitro, including, BEZ235, wortmannin, tuhulysin, lenalidomide, AS1517499, and BIRB796. The concentration of IL-10 was proportional to the absorbance at 492 nm. The black dotted line in each Figure indicated the IL-10 level of untreated control. The black solid line indicated IL-10 level of background. The absorbance at 492 nm for every sample was plotted vs concentrations of tested drugs from 0.1 μM to 100 μM.

As shown in FIG. 13, by comparing the effect of three PI3K inhibitors on decreasing the production of IL-10 by TAMs/MDSCs in vitro, it was found that GDC-0980 is the best candidate that can efficiently decrease IL-10 produced by TAMs/MDSCs.

### Example 7: Nitric Oxide Assay

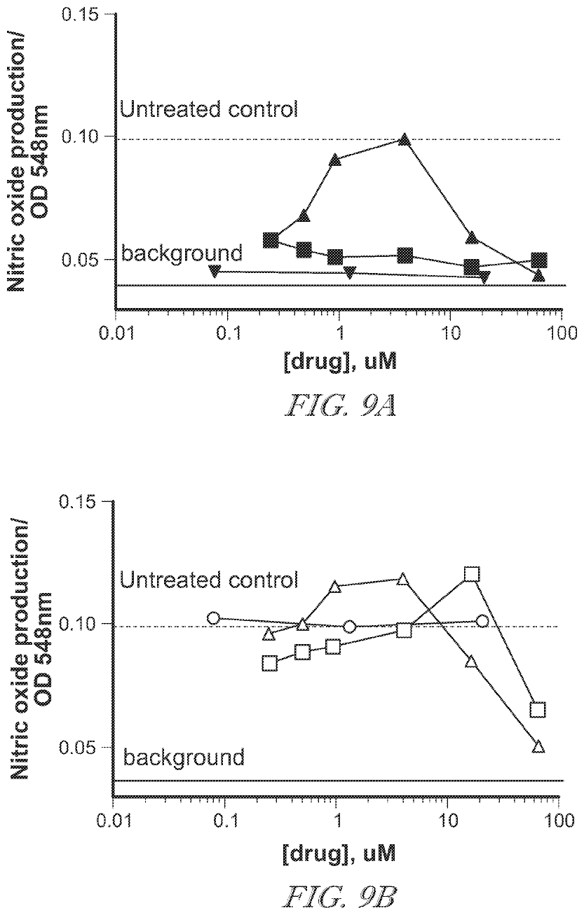

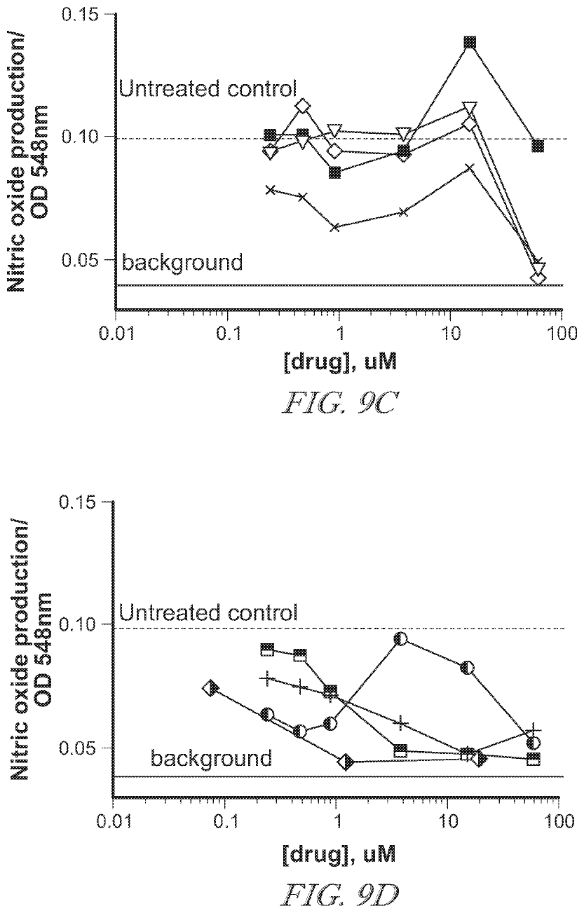

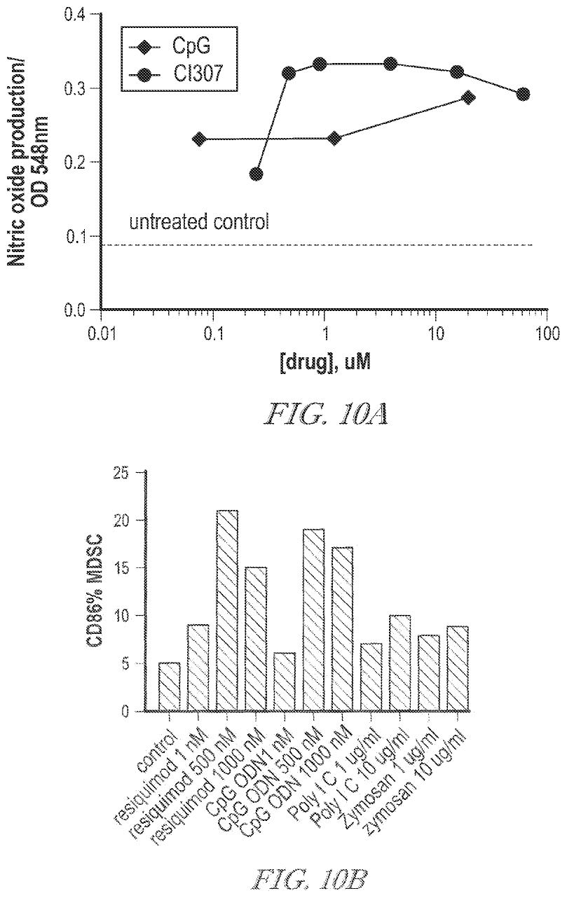

Nitric oxide production was measured with Greiss reagent as reported in Je-In Youn, Srinivas Nagaraj, Michelle Collazo, and Dmitry I. Gabrilovich, Subsets of Myeloid-Derived Suppressor Cells in Tumor Bearing Mice′, J Immunol. 2008 Oct. 15; 181(8): 5791-5802. Briefly, after in vitro incubation of TAMs/MDSCs with different drugs, 50 μL of supernatant from each well was transferred into a 96-well plat bottom clear plate. 20 μL of Greiss reagent and 30 μL of DI water were added to each well with 50 μL of supernatant. The reaction solution was kept in the dark at room temperature for 30 min prior to a plate reader measurement. The absorbance at 548 nm is correlated to concentration of nitric oxide produced by TAMs/MDSCs. Results are shown in FIG. 9, FIG. 10, FIG. 11 and FIG. 14.

As shown in FIG. 9, it was found that several drugs that can efficiently decrease nitric oxide production by TAMs/MDSCs in vitro, including BEZ235, wortmannin, AMT, methotrexate, tubulysin, AS1517499, everolimus, and BIRB796. The concentration of nitric oxide was proportional to the absorbance at 548 nm. The black dotted line in each Figure indicated the nitric oxide level of untreated control. The black solid line indicated the nitric oxide level of background. The absorbance at 548 nm for every sample was plotted vs concentrations of tested drugs from 0.1 μM to 100 μM.

As shown in FIG. 10 shows dramatically increased production of nitric oxide and upregulation of CD86 in vitro after co-culturing TAMs/MDSCs with different TLR agonists and indicates reprogramming TAMs/MDSCs to M1 macrophages with anti-tumor functions.

As shown in FIG. 11, in order to test the potency of newly synthesized TLR7 agonist (TLR7A) on affecting nitric oxide production by TAMs/MDSCs, TLR7A and C1307 were co-cultured with TAMs/MDSCs at different concentrations. From FIG. 11, it could be found that TLR7A is more efficient at increasing nitric oxide than a commercially available TLR7 agonist (C1307).

As shown in FIG. 14, by comparing the effect of three PI3K inhibitors at decreasing the production of nitric oxide by TAMs/MDSCs in vitro, it was found that GDC-0980 is the best candidate that can efficiently decrease nitric oxide produced by TAMs/MDSCs.

### Example 8: Statistical Analysis

The statistical significance between values was determined by Student's t-test. All data were expressed as the mean±SD. Probability values of p<0.05 were considered significant.

### Example 9: Ratio of M1 to M2 Macrophages

The ratio of M1 to M2 macrophages (F4/80+CD86+: F4/80+CD206+) was tested in groups of untreated control, FA-TLR7 agonist, FA-PI3K inhibitor, FA-Tubulysin and combination as well as competition groups.

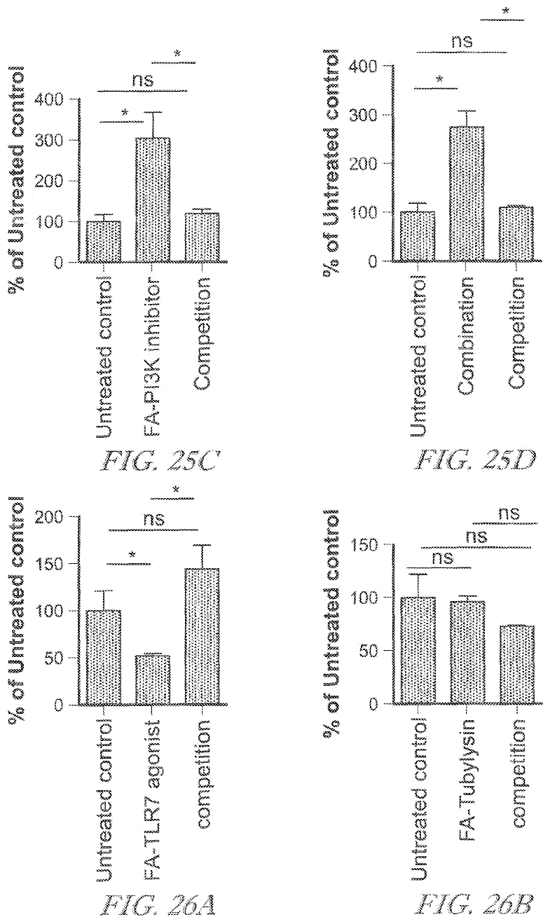

As described in the previous methods part, after tumor digestion at the end of therapeutic study, isolated cells were stained by F4/80 macrophage marker and M1 (CD86), M2 (CD206) markers. The ratio of M1 to M2 macrophages in 4T1 solid tumor were studied and summarized in FIG. 25. Macrophages in a tumor environment have been considered as a mainly M2 macrophage function, which can support tumor growth and suppress the immune response. On the other hand, M1 macrophages have been considered to be able to eliminate tumor cells and stimulate an anti-cancer immune response. Therefore, to study the M1 to M2 macrophage ratio is very important for targeting FR-β positive TAMs/MDSCs. As shown in FIG. 25, M1 to M2 macrophages ratio (F4/80+CD86+ cell population to F4/80+CD206+ cell population) from treatment and competition groups were compared with the ratio from untreated control. As a result, the ratio in three treatment groups (FA-TLR7 agonist, FA-PI3K inhibitor and combination) dramatically increased compared with untreated control and this effect could be competed by extra addition of competitor (FA-PEG-NH₂). Therefore, it could be concluded that by targeting FR+ TAMs/MDSCs in 4T1 solid tumor, the three classes of FA-conjugated MDSCs are able to convert immunosuppression M2 macrophages environment to an anti-cancer M1 macrophages environment, which would contribute to the slow growth of a tumor.

### Example 10: MDSCs Population

The MDSCs population (CD11b+Gr1+) was tested in groups of untreated control, FA-TLR7 agonist, FA-PI3K inhibitor, FA-Tubulysin and combination as well as competition groups.

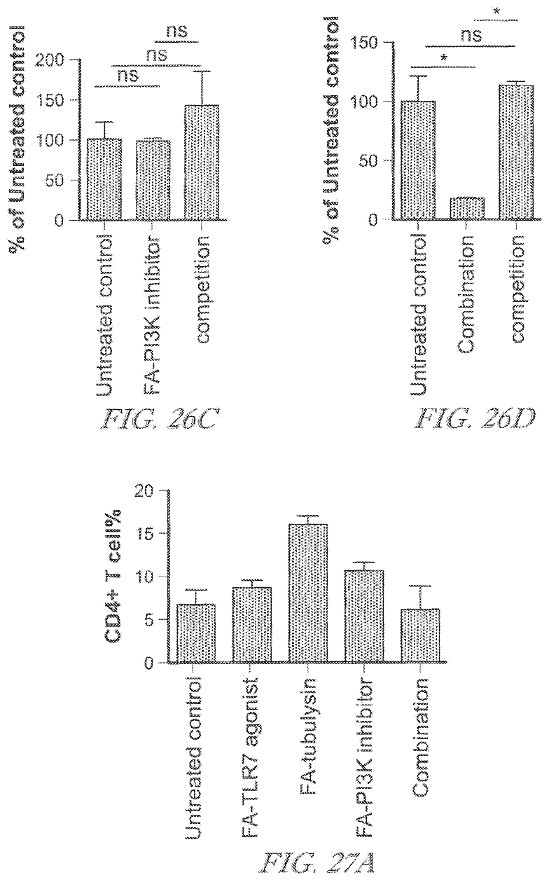

As described in previous methods part, after tumor digestion at the end of therapeutic study, isolated cells were stained by MDSCs markers CD11b+Gr1+, see FIG. 26. Only FA-TLR7 agonist and combination groups showed dramatically decreased MDSCs population. MDSCs population in treatment groups of FA-Tubulysin and FA-PI3K inhibitor showed no difference compared with untreated control and competition group. The decreasing of MDSCs population in TLR7 agonist treatment (FA-TLR7 agonist and combination groups) might be a result of reprograming MDSCs to a function of inhibiting tumor survival, which might cause a phenotype change of MDSCs. Although in vitro data indicated the toxicity of tubulysin on TAMs/MDSCs, in vivo tumor environment might be able to suppress the killing function of tubulysin since tumor cells might be able to release growth factors and cytokines that can support MDSCs survival with the existence of toxic tubulysin. As a result, the population of MDSCs for FA-tubulysin treatment did not change. However, by combining results in FIGS. 24, 25 and 26, it could be found that the function of TAMs/MDSCs (arginase level) and tumor environment (M1 to M2 macrophage ratio) in FA-tubulysin and FA-PI3K inhibitor groups have been modified even without changing the phenotype of MDSCs.

### Example 11: Percentages of CD4 and CD8 T Cell Populations

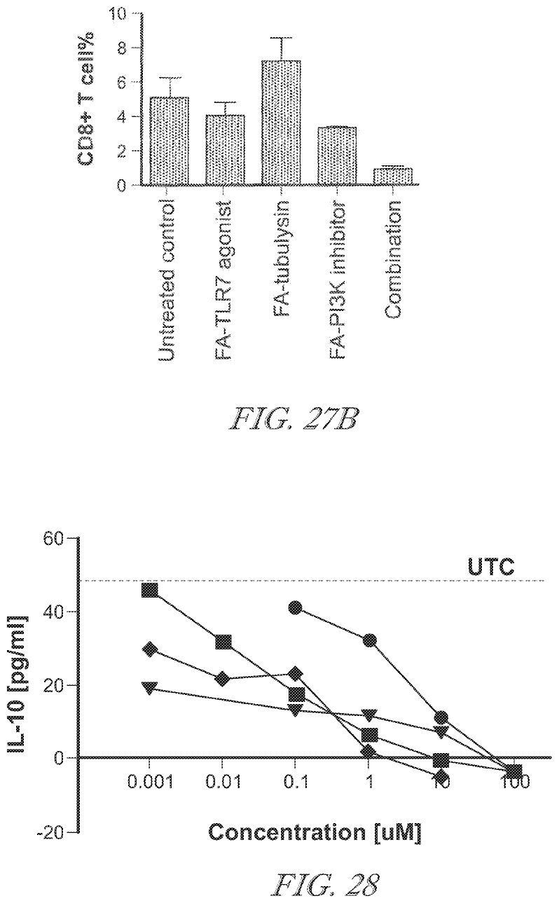

Percentages of CD4 and CD8 T cell populations were tested in live cells isolated from 4T1 solid tumors in groups of untreated control, FA-TLR7 agonist, FA-PI3K inhibitor, FA-Tubulysin, combination as well as competition groups (See FIG. 27).

Folate SMDCs treatment has more significant effect on increasing the population of CD4+ T cells than on increasing CD8+ T cells. It should be mentioned that since PI3K is important in T cell proliferation and activation, both CD4+ and CD8+ T cell in combination groups showed no difference or decreased population compared with untreated control.

### Example 12: In Vivo Studies

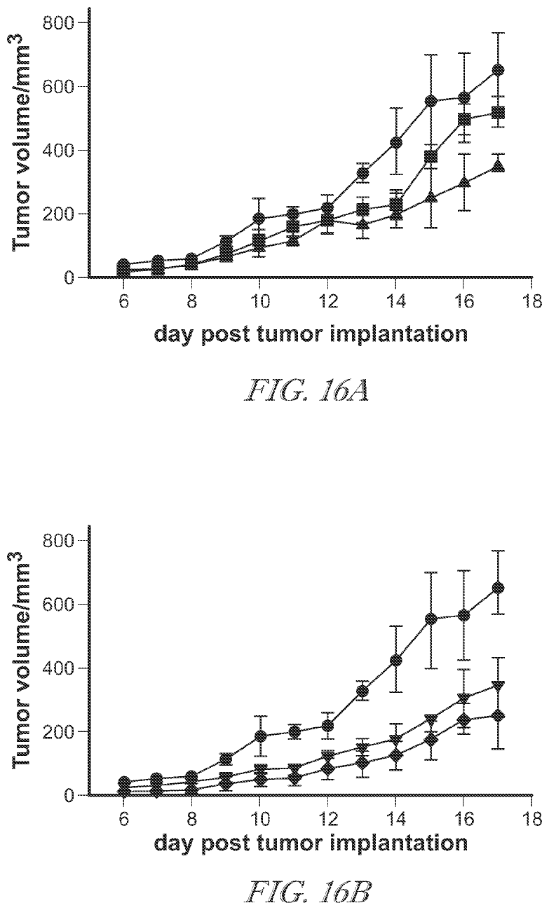

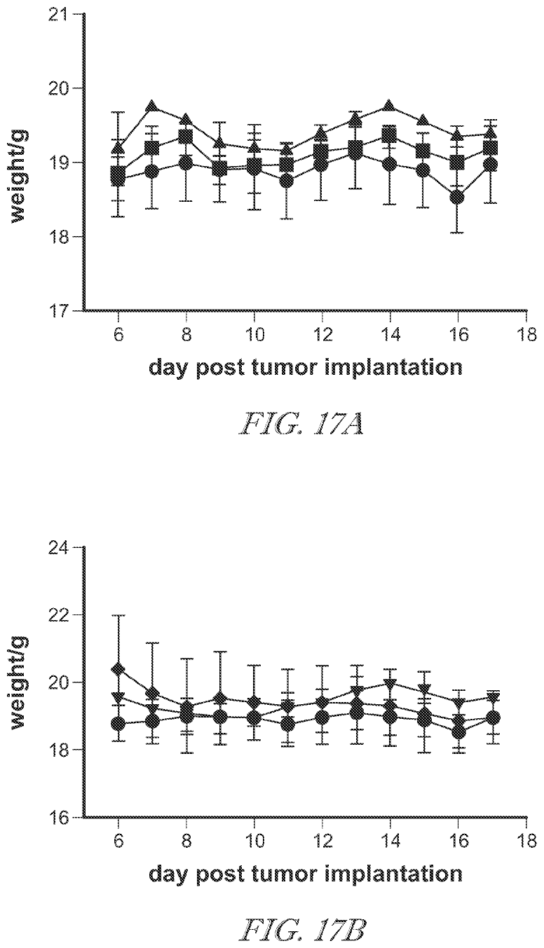

A dose study of FA-TLR7A was performed in 4T1 solid tumor model with 2 mice per group. Treatment was conducted by i.v. injection of different doses of FA-TLR7A for 5 days per week starting at day 6 after tumor implantation (subcutaneous, 0.05 million cells/mouse). Treatment was continued for 2 weeks. Tumor volume was measured every day. From this study, it could be seen that by targeting TAMs/MDSCs through folate receptor-β with TLR7 agonist, tumor growth was slowed down especially in groups of 5 nmol, 10 nmol and 20 nmol. Results are shown in FIGS. 16 and 17.

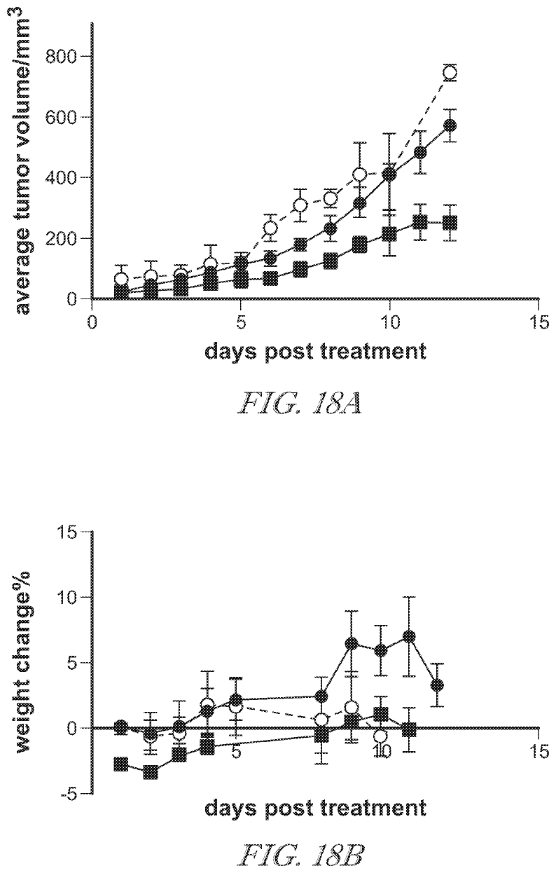

A therapeutic study of FA-TLR7 agonist was performed in 4T1 solid tumor model with 3 mice per group. Treatment was conducted by i.v. injection of 100 μl of 10 nmol FA-TLR7 agonist in PBS for 5 days per week starting at day 6 after tumor implantation (subcutaneous, 0.05 million cells/mouse). Treatment was continued for 2 weeks. Competition group was conducted at the same schedule by co-injection of 200 times more competitors (FA-PEG-NH₂) with 10 nmol of FA-TLR7 agonist. The total injection volume was 100 μl. Tumor volume was measured every day. From this study, it could be seen that by targeting TAMs/MDSCs through folate receptor-β with TLR7 agonist, tumor growth was slowed down. And this effect can be competed by adding extra FA-PEG-NH₂, which confirmed that the anti-cancer activity of FA-TLR7 agonist was mediated through FR-β. Results are shown in FIG. 18.

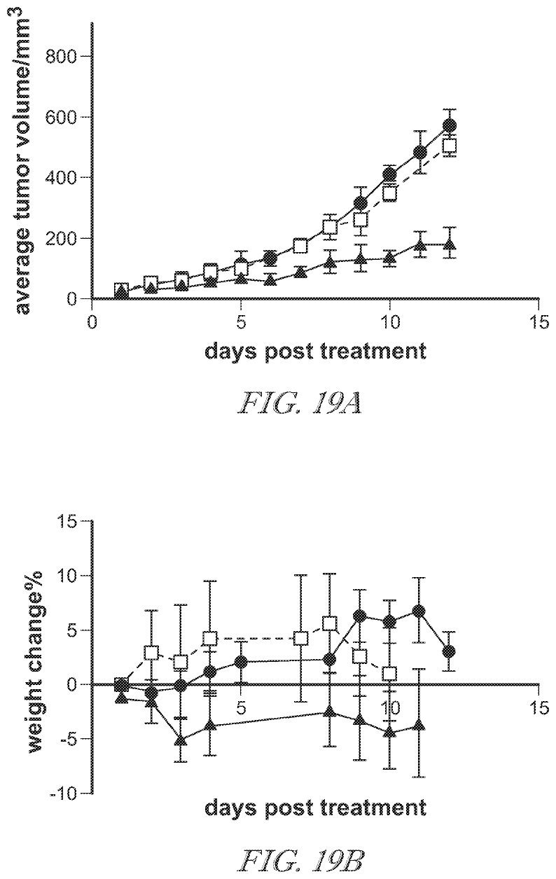

A therapeutic study of FA-tubulysin was performed in 4T1 solid tumor model with 3 mice per group. Treatment was conducted by i.v. injection of 100 μl of 30 nmol FA-tubulysin in PBS for 5 days per week starting at day 6 after tumor implantation (subcutaneous, 0.05 million cells/mouse). Treatment was continued for 2 weeks. Competition group was conducted at the same schedule by co-injection of 200 times more competitors (FA-PEG-NH₂) with 30 nmol of FA-tubulysin. The total injection volume was 100 μl. Tumor volume was measured every day. From this study, it could be seen that by targeting TAMs/MDSCs through folate receptor-β with tubulysin, tumor growth was slowed down. And this effect can be completed by adding extra FA-PEG-NH₂, which confirmed that the anti-cancer activity of FA-tubulysin was mediated through FR-β. Results are shown in FIG. 19.

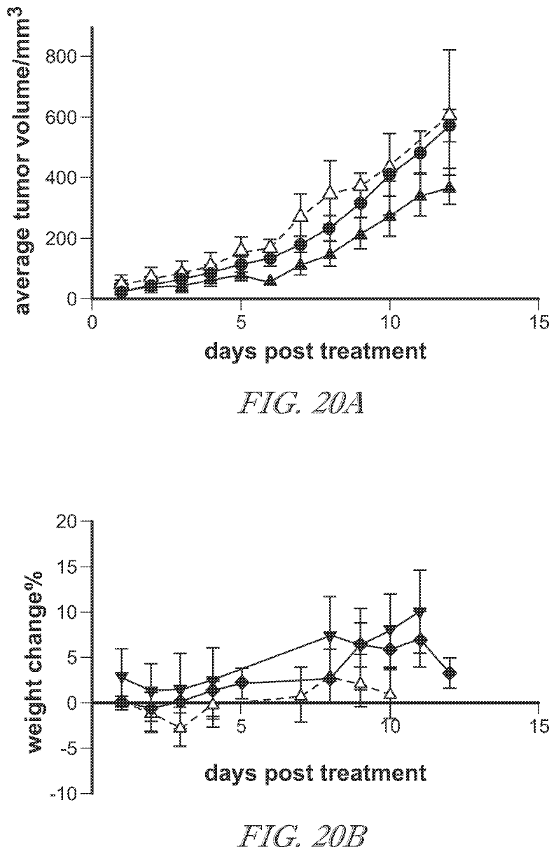

A Therapeutic study of FA-PI3K inhibitor was performed in 4T1 solid tumor model with 3 mice per group. Treatment was conducted by i.v. injection of 100 μl of 10 nmol FA-PI3K inhibitor in PBS for 5 days per week starting at day 6 after tumor implantation (subcutaneous, 0.05 million cells/mouse). Treatment was continued for 2 weeks. Competition group was conducted at the same schedule by co-injection of 200 times more competitors (FA-PEG-NH₂) with 10 nmol of FA-PI3K inhibitor. The total injection volume was 100 μl. Tumor volume was measured every day. From this study, it could be seen that by targeting TAMs/MDSCs through folate receptor-β with PI3K inhibitor, tumor growth was slowed down. And this anti-cancer effect can be competed by adding extra FA-PEG-NH₂, which confirmed that the anti-cancer activity of FA-PI3K inhibitor was mediated through FR-β. Results are shown in FIG. 20.

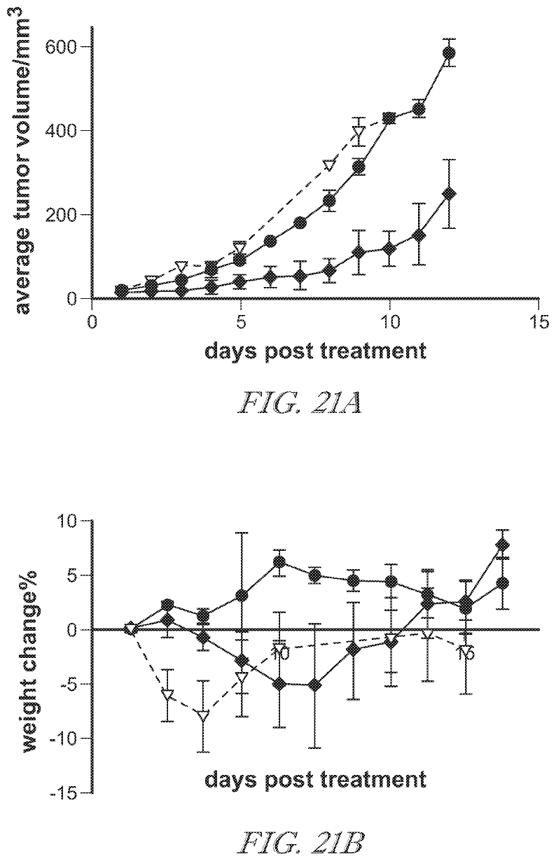

A combination therapeutic study of FA-TLR7 agonist and non-targeted PI3K inhibitor (BEZ235) was performed in 4T1 solid tumor model with 3 mice per group. Treatment was conducted by i.v. injection of 100 μl of 10 nmol FA-TLR7 agonist in PBS combined with oral dosing BEZ235 of 0.27 mg per mouse for 5 days per week starting at day 6 after tumor implantation (subcutaneous, 0.05 million cells/mouse). Treatment was continued for 2 weeks. Competition group was conducted at the same schedule by co-injection of 200 times more competitors (FA-PEG-NH₂) with 10 nmol of FA-TLR7 agonist combined with oral dosing BEZ235 of 0.27 mg per mouse. The total injection volume was 100 μl. Tumor volume was measured every day. From this study, it could be seen that by combination FA-TLR7 agonist with non-targeted PI3K inhibitor, tumor growth was significantly slowed down. And this effect can be competed by adding extra FA-PEG-NH₂, which confirmed that the anti-cancer activity of combination treatment was mediated through FR-β. However, by introducing PI3K inhibitor, BEZ235, certain toxicity could be observed at early dosing as decreasing of animal weight. Results are shown in FIG. 21.

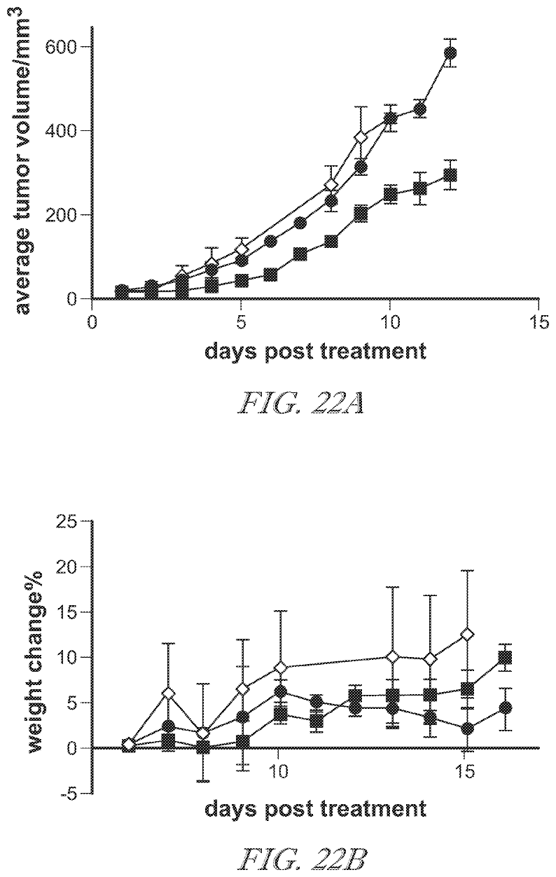

An in vivo therapeutic study of FA-TLR7 agonist was performed as previous mentioned. Non-targeted therapy of PI3K inhibitor (BEZ235) was conducted at a similar dosing schedule by orally administration of 0.27 mg per mouse for 5 days per week. The study continued for 2 weeks. By comparing FIGS. 21 and 22, a synergistic effect on slowing tumor growth could be seen for combination treatment, which confirmed the previous in vitro study of synergistic effect on arginase production by co-culturing TAMs/MDSCs with TLR7 agonist and PI3K inhibitor. Results are shown in FIG. 22.

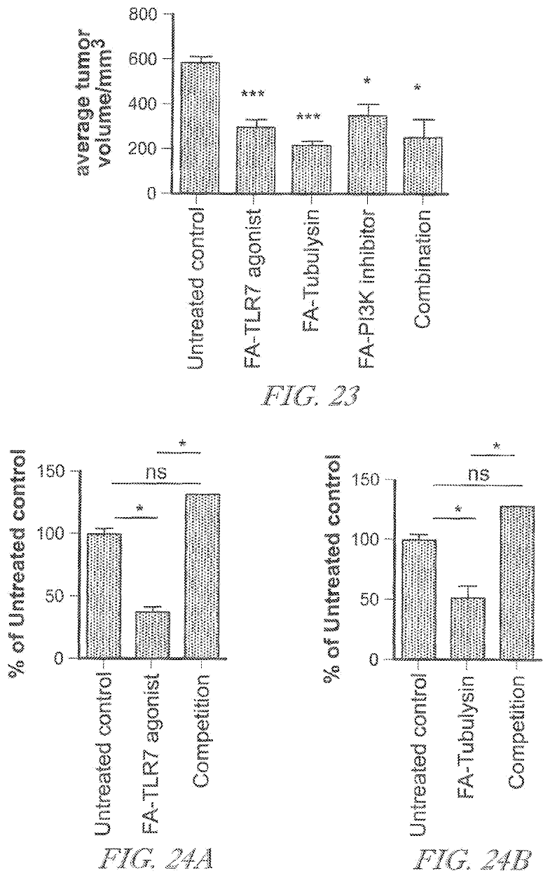

FIG. 23 shows the average tumor volume at the last day of treatment for therapeutic group.

### Example 13: In Vitro Induction of Human MDSCs from PBMCs

Human PBMCs from healthy donor were isolated by density gradient centrifugation following standard procedure:

Blood was dilute blood with PBS (1:2 dilutions). 15 ml of Ficoll was transferred to a 50 ml tube. 35 ml of diluted blood was carefully placed over the Ficoll medium. The tube was centrifuged at 400 g for 30 min, at 24° C. without brake. After the centrifuge stoped, the tube was carefully removed from the centrifuge while not disturbing the layering. The PBMCs were carefully remove from the tube and transferred to a new 50 mL conical tube. Isolated PBMCs were washed with PBS and centrifuged at 300 g for 10 min. The supernatant was decanted. The pellet was washed once again in PBS and centrifuged at 200 g for 15 min. The isolated PBMCs were counted with the hemocytometer.

Isolated PBMCs were further purified by adhesion in serum free medium for 4 hours at 37° C. at a density of 3×10⁶ cell/ml. After removing the suspension cells, adhered PBMCs were cultured in completed RPMI-1640 supplied with 10 ng/ml of IL-6 and GM-CSF for 7 days. Human MDSCs were then sorted by flow as CD33+ cells. Normal human macrophages were differentiated by co-culture PBMCs with completed RPMI-1640 medium for 7 days.

Human MDSCs were cultured with selected drugs for 2 days. The IL-10 production by MDSCs was measured and plotted vs drug concentrations. Human MDSCs showed similar response to these drugs with decreasing IL-10, which might contribute to the inhibition of immunosuppression of MDSCs. Results are shown in FIG. 28.

### Example 14: In Vitro Activation of Human T Cells and Inhibition of T Cell Suppression

Human PBMCs were isolated by density gradient centrifugation as mentioned in Example 13. Isolated PBMCs were re-suspended in 1 ml of PBS with 2% FBS and 1 mM EDTA in 15 ml tube with a concentration of 5×10⁷ cells/ml. A 50 μl of cocktail solution of Human T cell Enrichment Kit was added to the suspension. Cells were incubated for 10 min at RT. 50 μl of magnetic beads (Human T cell Enrichment Kit) were added and incubated for 5 min at RT. The tube with T cells and magnetic beads was placed into a magnet for 5 min at RT. The supernatant contained negatively selected human T cells which was collected and counted. Isolated T cells were cultured with 50 U/ml of IL-2 at a density of 1×10⁶ cells/ml for 3 days. The cell solution was then mixed well with a pipette and placed next to a magnet for 5 min to remove beads. The suspension was collected that contained activated human T cell for suppression assay.

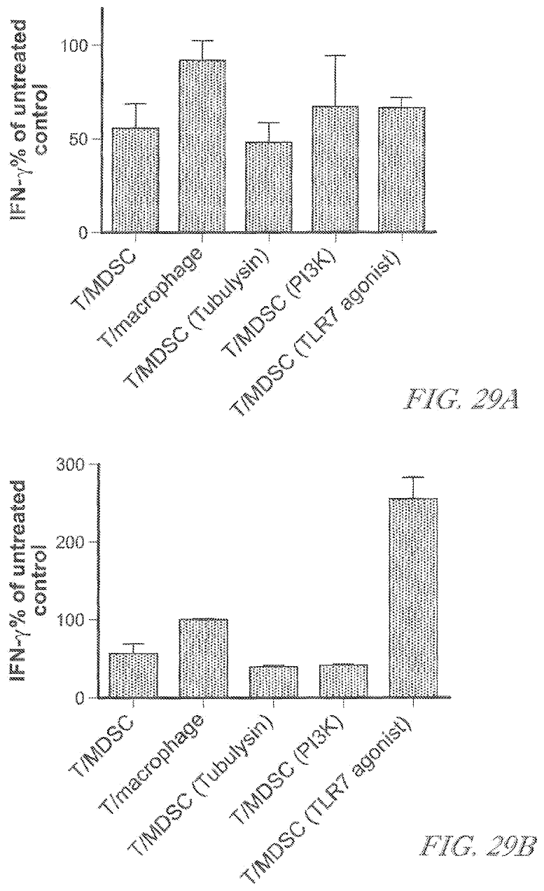

Human MDSCs, which were co-cultured with 3 classes of drugs at a concentration of 0.1 or 1 μM for 2 days, were mixed with activated human T cells at a ratio of 8:1 for 18 hours. The production of IFN-γ was measured as a T cell activation marker. Compared to macrophages, MDSCs showed 50% inhibition of T cell activation. For the drug concentration of 0.1 μM, there was no significant change in IFN-γ production from T cells. However, at the concentration of 1 TLR7 agonist treated MDSCs showed dramatically increased IFN-γ from T cells indicating that the suppression function of MDSCs might be inhibited or reversed by TLR7 agonist stimulation in vitro. Results are shown in FIG. 29.

### Example 15: Lung Metastasis Assay

Balb/c mice implanted with 4T1 cells were treated with three classes of FA-conjugates for 2 weeks (7 days/week) when the tumor size reached 50 mm³. After 2 weeks treatment, animals were euthanized and lungs were digested with 5 ml of collagenase IV PBS solution (1 mg/ml) for 2 hours at 37° C. The suspension was passed through a 70 μm cell strainer to obtain a single cell suspension. Cells were co-cultured with 10 ml of completed RPMI-1640 medium containing 60 μM of 6-thioguanine for 10 to 14 days. The medium was removed at the end of culture. Cells were fixed with 5 ml of methanol for 5 min at room temperature and were washed with DI water once. 5 ml of methylene blue (0.03%, v/v) was added to stain cells for 5 min at room temperature. After being washed with water, cells were air dried for evaluation of metastasis.

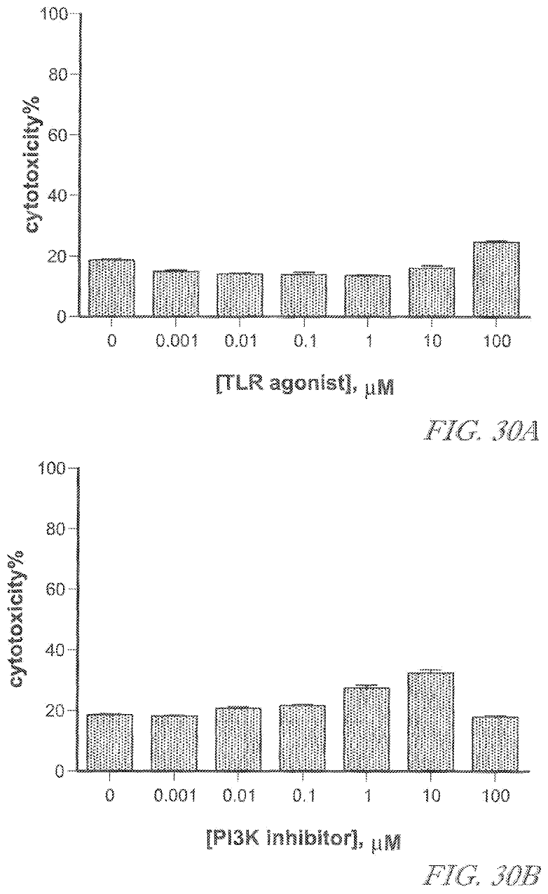

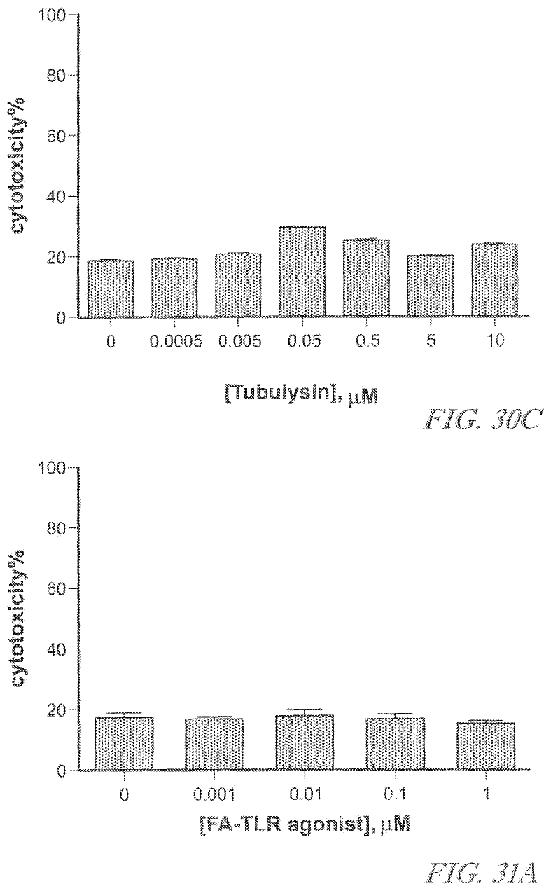

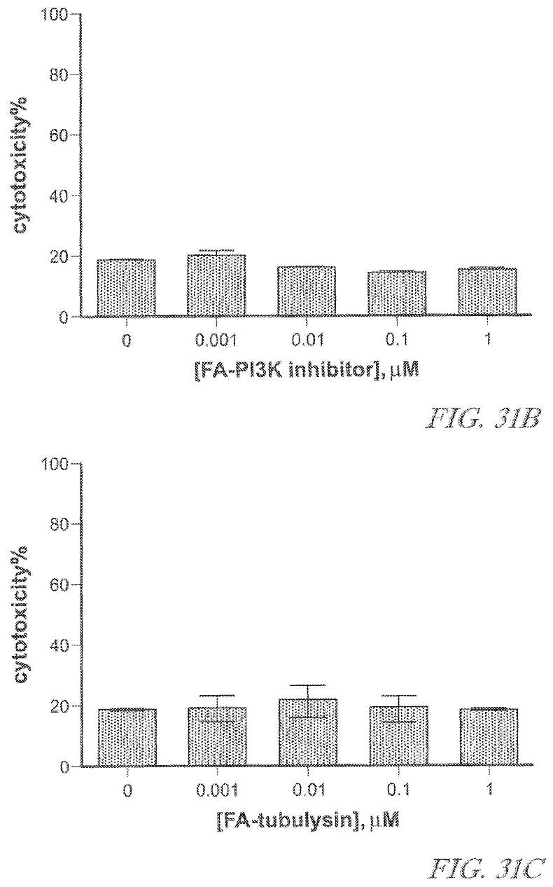

4T1 cells show resistance to both FA-conjugates and released drugs. Therefore, it could be thought that the in vivo anti-cancer activities of FA-conjugates should be attributed to the targeting of FR-β positive myeloid cells by inhibiting or reprogramming the immunosuppression function. Results are shown in FIG. 30 and FIG. 31.

The administration of FA-conjugates was changed from 5 days per week to 7 days per week in order to see whether an improved therapeutic effect could be achieved. Continuous administration of FA-conjugates to 4T1 solid tumor can decrease tumor growth. Results are shown in FIG. 32.

By targeting MDSCs/TAMs, the arginase level was dramatically decreased in three treatment groups that might contribute to the elimination of T cell suppression. Results are shown in FIG. 33.

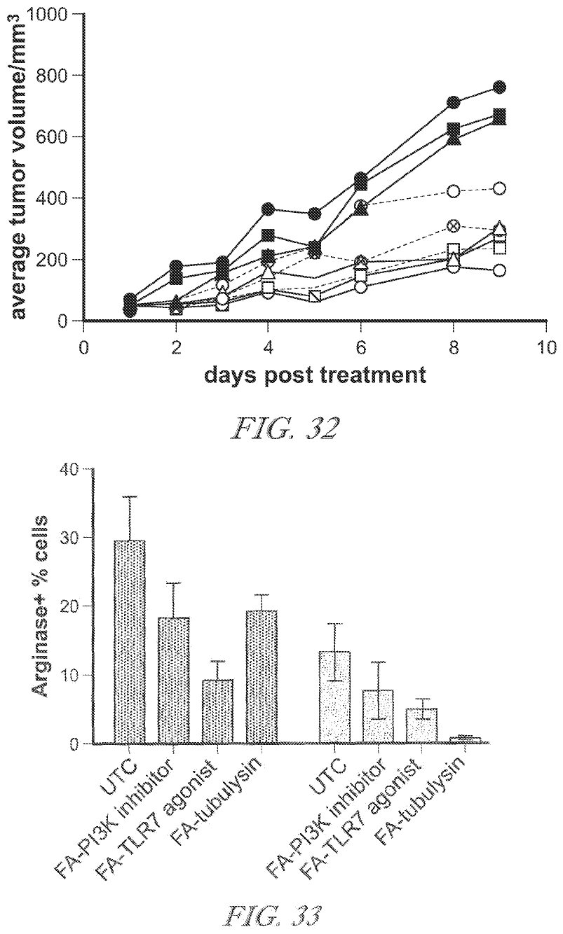

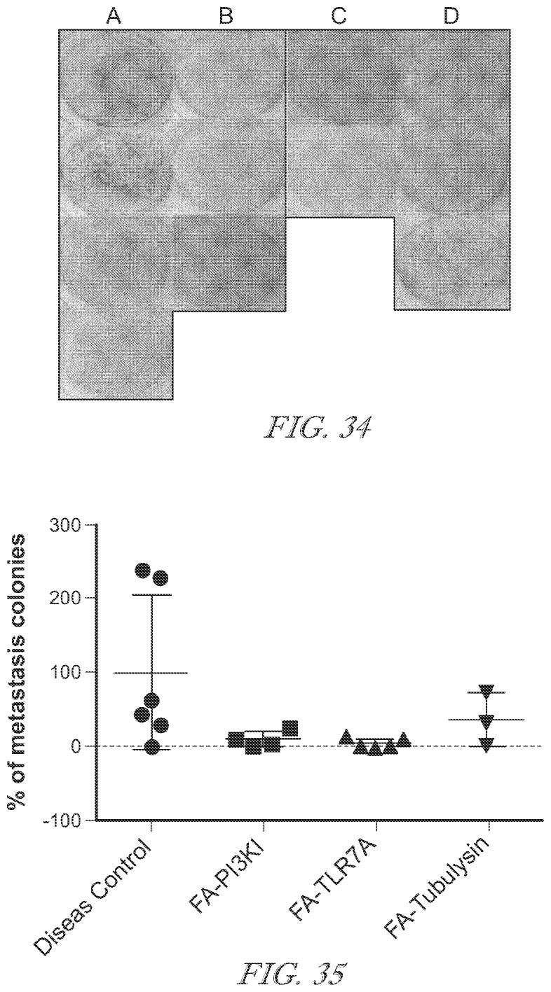

MDSCs are directly implicated in the promotion of tumor metastases by participating in the formation of pre-metastatic niche, promoting angiogenesis and tumor cell invasion. Our hypothesis is that elimination of MDSCs/TAMs would prevent cancer metastasis. Previous study showed that TLR7 stimulation/PI3K inhibition can either decrease MDSCs population, or convert immunosuppression MDSCs/TAMs to M1 like macrophages, or inhibit immunosuppression function such as arginase and IL-10. As a result, T cell activation might be promoted and systemic immunity would be improved. Metastasis data showed decreased lung metastasis in treatment groups compare with untreated disease control. Results are shown in FIGS. 34 and 35.

### Example 16. Survival Study

Balb/c mice were implanted with 5×10⁴ cells s.q. Treatment by FA-conjugates was started when the tumor size reached ˜50 mm³ and continued for 2 weeks as 7 days per week. Tumor was removed by surgery when the size reached 150-200 mm³. Animal survival was monitored.

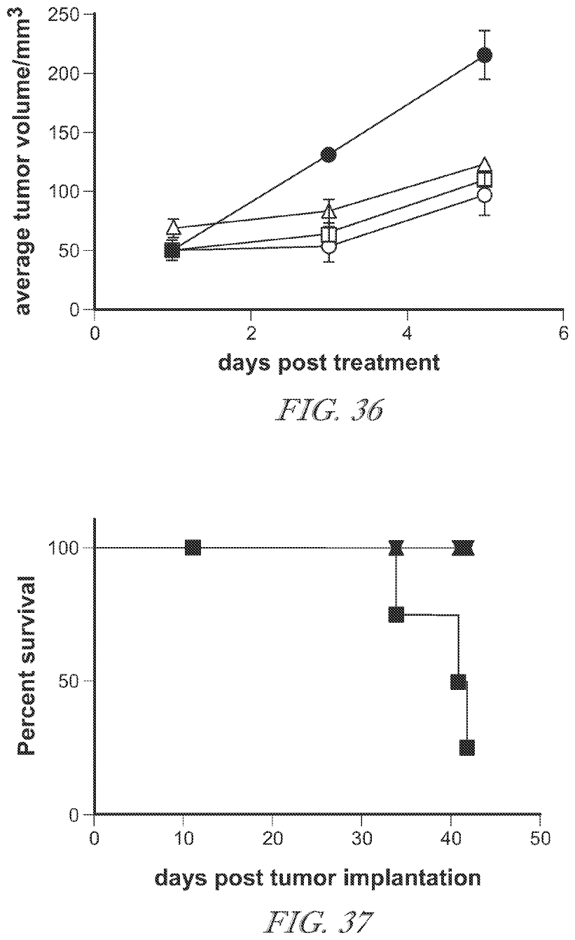

Mice carrying 4T1 solid tumor were treated with FA-conjugates to target MDSCs/TAMs when the tumor size reached 50 mm³. Tumor was removed when the size reached 150-200 mm³. The treatment was continued for a total 2 weeks (7 days per week). The survival of mice was monitored. It could be seen that after elimination of immunosuppression function of MDSCs/TAMs, animal survival was significantly increased. This study is still on going to monitor animal survival and blood serum cytokines. Results are shown in FIG. 36 and FIG. 37.

### CHEMISTRY EXAMPLES

### Example 1: Synthesis of TLR7 Agonist (TLR7A)

TLR7 agonist (TLR7A) was synthesized following the procedure in Scheme 1 as reported by Nikunj M. Shukla, Cole A. Mutz, Subbalakshmi S. Malladi, Hemamali J. Warshakoon, Rajalakshmi Balakrishna, and Sunil A. David, ‘Regioisomerism-dependent TLR7 agonism and antagonism in an imidazoquinoline; Structure-Activity Relationships in Human Toll-Like Receptor 7-Active Imidazoquinoline Analogues’, J Med Chem. 2012 Feb. 9; 55(3): 1106-1116.

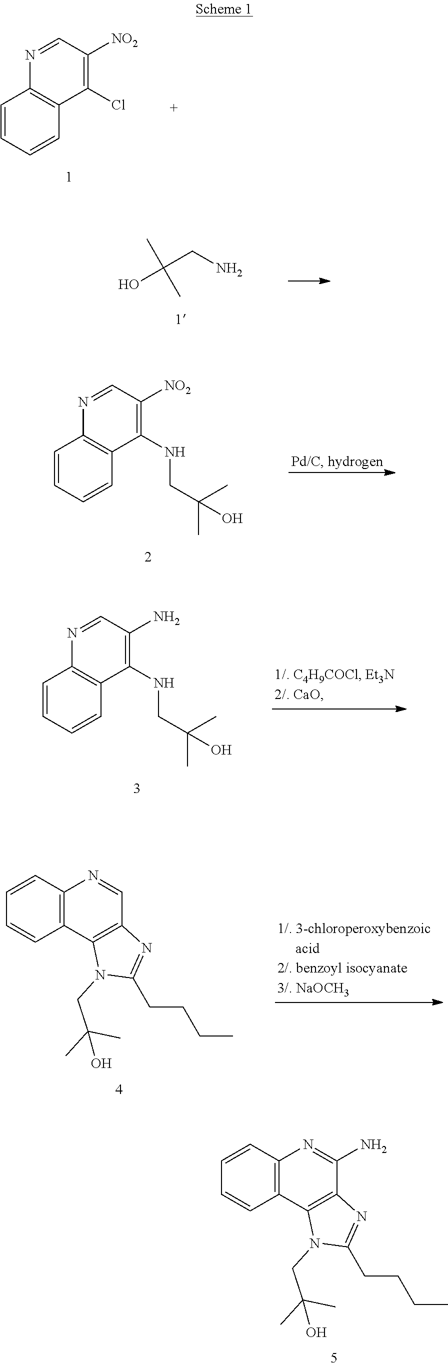

### Step 1: Synthesis of 1-amino-2-methylpropan-2-ol (Compound 1′)

2,2-dimethyloxirane (0.1 g, 1.388 mmol) was added dropwise to 20 mL ice cooled solution of ammonium hydroxide. The reaction mixture was stirred for 12 hours at room temperature. The solvent was removed under vacuum and the residue was dissolved in methanol. Di-tert-butyl dicarbonate (0.75 g, 3.47 mmol) was added to the reaction mixture and stirred for 4 hours. The mixture was purified using column chromatography (24% EtOAc/hexane) to obtain tert-butyl 2-hydroxy-2-methylpropylcarbamate. The pure tert-butyl 2-hydroxy-2-methylpropylcarbamate was dissolved in 5 mL of trifluoroacetic acid and stirred for 35 minutes. The solvent was removed under reduced pressure to afford 1-amino-2-methylpropan-2-ol as the trifluoroacetate salt 1′. ¹H NMR 500 MHz (500 MHz, CDCl3, δ in ppm): δ 8.62 (s, 2H), 3.02 (d, 2H), 2.06-2.04 (m, 2H), 1.37-1.34 (s, 6H).

### Step 2: Synthesis of 2-methyl-1-(3-nitroquinolin-4-ylamino)propan-2-ol (compound 2)

The trifluoroacetate salt of 1-amino-2-methylpropan-2-ol (compound 1′) (450 mg, 2.4 mmol) was added to the solution of 4-chloro-3-nitroquinoline (compound 1) (250 mg, 1.2 mmol) and Et₃N (0.5 ml, 3 mmol) in 4:1 mixture of toluene and 2-propanol. The mixture was heated to 70° C. for half an hour until a solid started precipitating. The reaction mixture was then cooled, filtered, washed with toluene/2-propanol (7:3), ether and cold water. The residue was dried at 80° C. to obtain 2-methyl-1-(3-nitroquinolin-4-ylamino)propan-2-ol (compound 2). LCMS: [M+H]⁺ m/z=261.

### Step 3: Synthesis of 1-(3-aminoquinolin-4-ylamino)-2-methylpropan-2-ol (compound 3)

2-Methyl-1-(3-nitroquinolin-4-ylamino)propan-2-ol (compound 2) (450 mg, 1.72 mmol) was dissolved in methanol and hydrogenated over Pd/C as catalyst with hydrogen balloon for 4 hours. The solution was then filtered using celite, followed by evaporation of solvent under reduced pressure to afford 1-(3-aminoquinolin-4-ylamino)-2-ethylpropan-2-ol (compound 3). LCMS: [M+H]⁺ m/z=231. ¹H NMR 500 MHz (CDCl3, δ in ppm): δ 8.12 (s, 1H), 7.61-7.58 (m, 1H), 7.48-7.40 (m, 2H), 4.90 (s, 2H), 3.47 (2H), 1.35-1.21 (s, 6H).

### Step 4: Synthesis of 1-(4-Amino-2-butyl-1H-imidazo[4,5-c]quinolin-1-yl)-2-methylpropan-2-ol (Compound 5, TLR7A)

To a solution of compound 3 (100 mg, 0.43 mmol) in anhydrous THF were added triethylamine (66 mg, 0.65 mmol) and valeryl chloride (62 mg, 0.52 mmol). The reaction mixture was then stirred for 6-8 hours, followed by removal of the solvent under vacuum. The residue was dissolved in EtOAc, washed with water and brine, and then dried over Na/SO₄ to obtain the intermediate amide compound. This was dissolved in MeOH, followed by the addition of calcium oxide, and was heated in microwave at 110° C. for 1 hour. The solvent was then removed and the residue was purified using column chromatography (9% MeOH/dichloromethane) to obtain the compound 4 (58 mg). To a solution of compound 4 in a solvent mixture of MeOH:dichloromethane:chloroform (0.1:1:1) was added 3-chloroperoxybenzoic acid (84 mg, 0.49 mmol), and the solution was refluxed at 45-50° C. for 40 min. The solvent was then removed and the residue was purified using column chromatography (20% MeOH/dichloromethane) to obtain the oxide derivative (55 mg). This was then dissolved in anhydrous dichloromethane, followed by the addition of benzoyl isocyanate (39 mg, 0.26 mmol) and heated at 45° C. for 15 min. The solvent was then removed under vacuum, and the residue was dissolved in anhydrous MeOH, followed by the addition of excess sodium methoxide. The reaction mixture was then heated at 80° C. for an hour. The solvent was removed under vacuum, and the residue was purified using column chromatography (11% MeOH/dichloromethane) to obtain the compound 5. LCMS: [M+H]⁺ m/z=312. ¹H NMR 500 MHz (CDCl3, δ in ppm): δ 8.16-8.15 (d, 1H), 7.77-7.46 (d, 1H), 7.46-7.43 (m, 1H), 7.33-7.26 (m, 1H), 3.00-2.97 (m, 2H), 1.84-1.78 (m, 2H), 1.47-1.41 (m, 2H), 1.36 (s, 6H), 0.98-0.95 (m, 3H).

### Example 2: Synthesis of Heterobifunctional Disulfide Linker (Compound 7)

Heterobifunctional Disulfide Linker (compound 7) was synthesized as shown in Scheme 2 following the procedure described by Satyam A., ‘Design and synthesis of releasable folate-drug conjugates using a novel heterobifunctional disulfide-containing linker’, Bioorg. Med. Chem. Lett. 2008 Jun. 1; 18(11):3196-9.

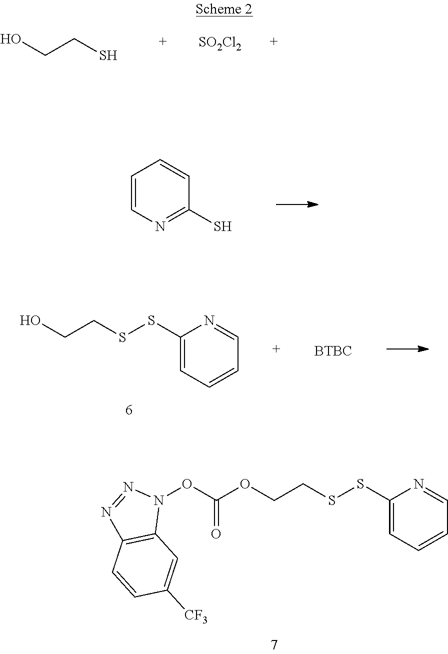

### Step 1: Synthesis of Heterobifunctional Disulfide Linker (Compound 7)

Sulfuryl chloride (25 mL of 1M solution in methylene chloride) was added over a period of 20 min to a stirred solution of 2-mercapropyridine (2.5 g, 22.5 mmol) in 25 mL of dry methylene chloride at 0-5° C. under nitrogen atmosphere. Yellow solid precipitated out. The mixture was stirred at room temperature for 2 hours and concentrated by rotavap, and the granular solid thus obtained was taken up in 50 mL of dry methylene chloride and cooled in ice-bath. To this stirred suspension at 0-5° C. under nitrogen atmosphere was added as solution of 2-mercaptoethanol (1.7 mL, 24.2 mmol) in 30 mL of dry methylene chloride over 5 min. Initially, the suspension dissolved to form a clear solution. However, within 15-20 min, a light yellow granular solid started to separate. The mixture was stirred at room temperature for overnight. The precipitate was filtered, washed with HPLC grade methylene chloride, and dried in vacuum desiccator for a few hours. The free base of the compound (compound 6) can be liberated by mixing the suspension of its hydrochloride salt in methylene chloride with a slightly more than equimolar quantity of dimethylaminopyridine and passing the mixture through a short column of silica gel using 5% methanol in methylene chloride as eluant. A solution of compound 6 (free base, 1 g, 5.4 mmol) in 10 mL of acetonitrile was added over 2 min to a stirred solution of BTBC (2.5 g, 5.7 mmol) in 50 mL of acetonitrile at room temperature. The resulting mixture was stirred at room temperature for 38 hours. The mixture was concentrated in vacuo and the residue was partitioned between ethyl acetate (50 mL) and IN NaHCO₃ (25 mL). The organic layer was separated, washed further with IN NaHCO₃ (10 mL), dried (anhydrous Na₂SO₄, filtered and concentrated in vacuum to give the compound 7. LCMS: [M+H]⁺ m/z=416. NMR 500 MHz (CDCl3, δ in ppm): δ 8.38-8.32 (m, 3H), 8.09-8.07 (m, 1H), 7.77-7.75 (m, 1H), 7.70-7.69 (m, 1H), 7.14-7.13 (m, 1H), 4.81-4.78 (m, 2H), 3.33-3.31 (m, 2H).

### Example 3: Synthesis of BTBC (Compound 8)

BTBC was synthesized as shown in Scheme 3 following the procedure described by Takeda, K.; Tsuboyama, K.; Hoshino, M.; Kishino, M.; Ogura, H. ‘A Synthesis of a New Type of Alkoxycarbonylating Reagents from 1,1-Bis[6-(trifluoromethyl)benzotriazolyl] Carbonate (BTBC) and Their Reactions’, Synthesis, 1987, 557-560.

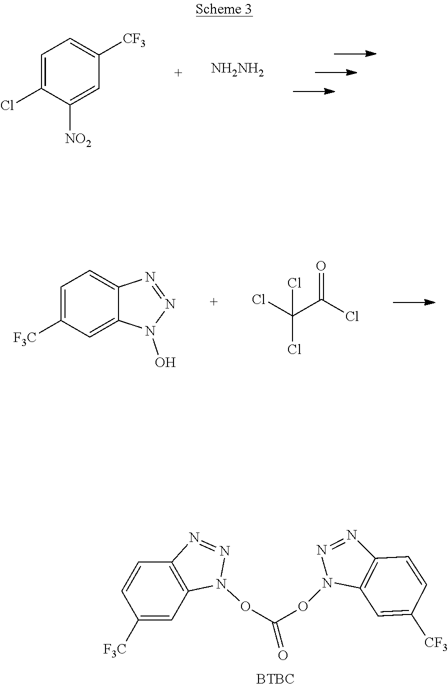 [SMILES: O=[N+]([O-])c1cc(C(F)(F)F)ccc1Cl ; "NH2NH2" ; On1nnc2ccc(C(F)(F)F)cc21 ; O=C(Cl)C(Cl)(Cl)Cl ; O=C(On1nnc2ccc(C(F)(F)F)cc21)On1nnc2ccc(C(F)(F)F)cc21 ; "BTBC"]

A mixture of 4-chloro-3-nitro-a,a,a-trifluorotoluene (2.5 g, 0.011 mol) and hydrazine hydrate (1.65 g, 0.033 mol) in 99% ethanol (20 mL) was refluxed for 24 hours. After removal of the solvent under reduced pressure, the residue was dissolved in 10% aqueous Na₂CO₃ solution. The solution was washed with ether to remove the starting material and acidified with concentrated HCl to precipitate the product, which was washed with water and dried to obtain 1-hydroxy-6-(trifluoromethyl)benzotriazol. To a stirred solution of 1-hydroxy-6-(trifluoromethyl)benzotriazol (1 g, 5 mmol) in dry ether (50 mL) was added trichloromethylchloroformate (0.26 g, 1.23 mmol) at room temperature. After 10 min, a further quantity of trichloromethylchloroformate (0.26 g, 1.23 mmol) was added to the mixture, refluxed gently for 1 hour, and the precipitate formed was collected and washed with dry ether. Almost pure crystals of BTBC are obtained. LCMS: [M+H]⁺ m/z=432.

### Example 4: Synthesis of Folic Acid-Cysteine (Compound 9) by Solid Phase Synthesis

H-Cys(Trt)-2-chlorotrityl resin (100 mg) was dispersed in 12 mL of dichloromethane and bubbled with argon for 10 min. After removing dichloromethane, 10 mL of DMF 10 mL was added and bubbled for 5 min. 5 mL of 20% piperidine in DMF solution was added three times for 10 min each. Resin was washed 3 times with 10 mL of DMF for 5 min each. 10 mL of isopropanol was added to wash resin 3 times for 5 min each. After drying in air for several minutes, free amine was tested by solid synthesis monitor kit with blue beads indicating completed deprotection of amine. Fmoc-Glu-OtBu (64 mg, 0.15 mol), DIPEA (0.105 mL, 0.6 mol), PyBOP (79 mg, 0.15 mol) dissolved in DMF were added to the beads in DMF solution. After reaction for 5-6 hours, repeated washing 3 times with DMF/IPA was performed. Deprotection of the amine was carried out by adding 5 mL of 20% piperidine DMF solution 3 times. After washing 3 times with DMF, 2 mL of DMF solution with N10-(trifluoroacetyl) pteroic acid (62 mg, 0.15 mol), DIPEA (0.105 ml, 0.6 mol), PyBOP (79 mg, 0.15 mol) was added to the beads in DMF solution. Reaction continued under argon for 5-6 hours. 8 mL of mixed solution of TFA/ethanedithiol/thiisoproylsilane/H₂O with volume ratio of 96.25/1.25/1.25/1.25 was added 3 times for 30 min each to cleave compound from resin. Trifluoroacetyl-protected compound 8 was purified through HPLC. Compound 8 was obtained after deprotection trifluoroacetyl group by ammonium solution (5 ml, 0.5 M) for 2 hours at room temperature. LCMS: [M+H]⁺ m/z=544.

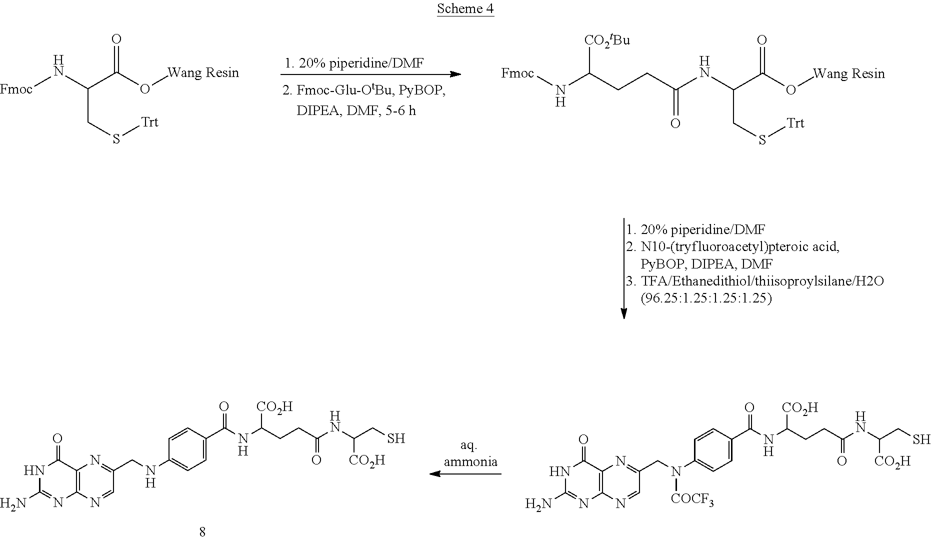

### Example 5: Synthesis of Folic Acid Conjugates of TLR7 Agonist (TLR7A)

Folic acid conjugate of TLR7 agonist (TLR7A) was synthesized as shown in Scheme 5.

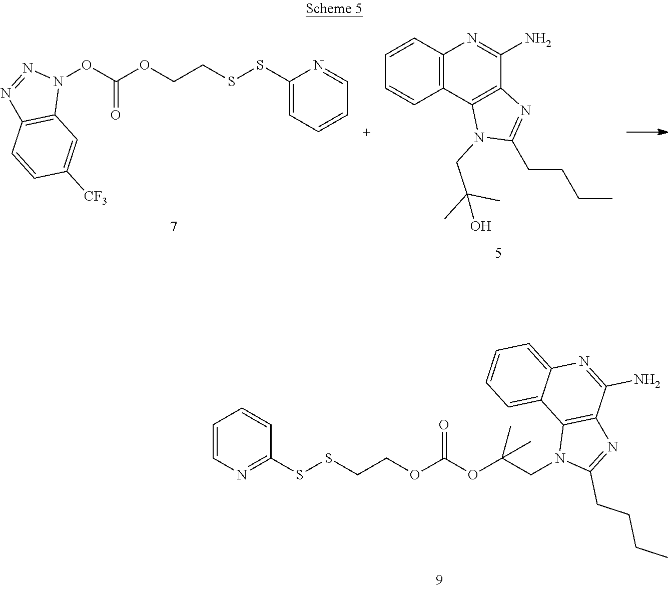

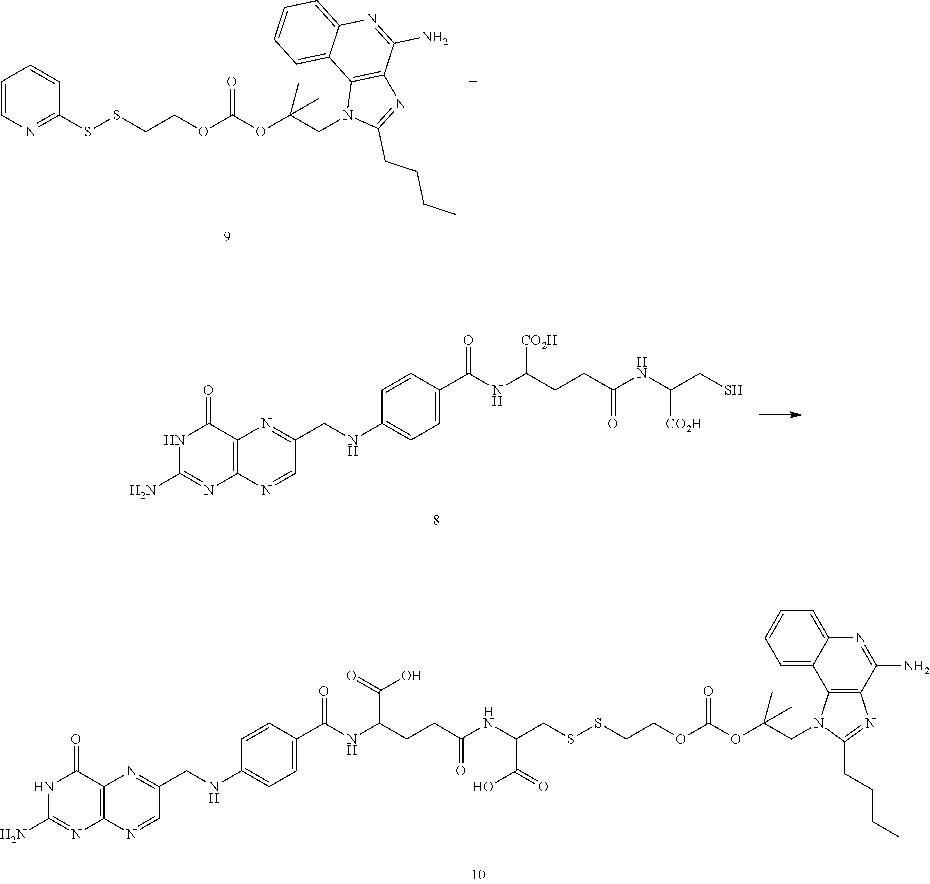 [SMILES: CCCCc1nc2c(N)nc3ccccc3c2n1CC(C)(C)OC(=O)OCCSSc1ccccn1 ; Nc1nc2ncc(CNc3ccc(C(=O)NC(CCC(=O)NC(CS)C(=O)O)C(=O)O)cc3)nc2c(=O)[nH]1 ; CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]

Heterobifunctional linker 7 (88 mg, 0.213 mmol) was added to a solution of compound 5 (33 mg, 0.106 mmol) and dimethylaminopyridine (39 mg, 0.319 mmol) in 4 mL of methylene chloride at room temperature under nitrogen atmosphere and the mixture was stirred at reflux temperature for 7 hours at which time TLC analysis of the mixture indicated >80% conversion. The mixture was concentrated and purified by column chromatography using 10% acetonitrile in methylene chloride as eluant. The pure product compound 9 was obtained as a light yellow solid. A solution of compound 8 (1 eq.) in DMSO was added in 3 portions at 20 min intervals to a solution of drug-linker intermediates compound 9 (1.0-1.5 eq.) in DMSO with dimethylaminopyridine (1 eq.). After 1-2 hours of stirring at RT under argon, LCMS analysis of the mixture indicated formation of the desired folate-drug conjugate (compound 10) as the major product. The mixture was purified by preparative HPLC. LCMS: [M+H]⁺ m/z=959.

### Example 6: Synthesis of FA-PI3K Inhibitor (Compound 12)

Folic acid conjugate of PI3K inhibitor (GDC-0980) was synthesized as shown in Scheme 6.

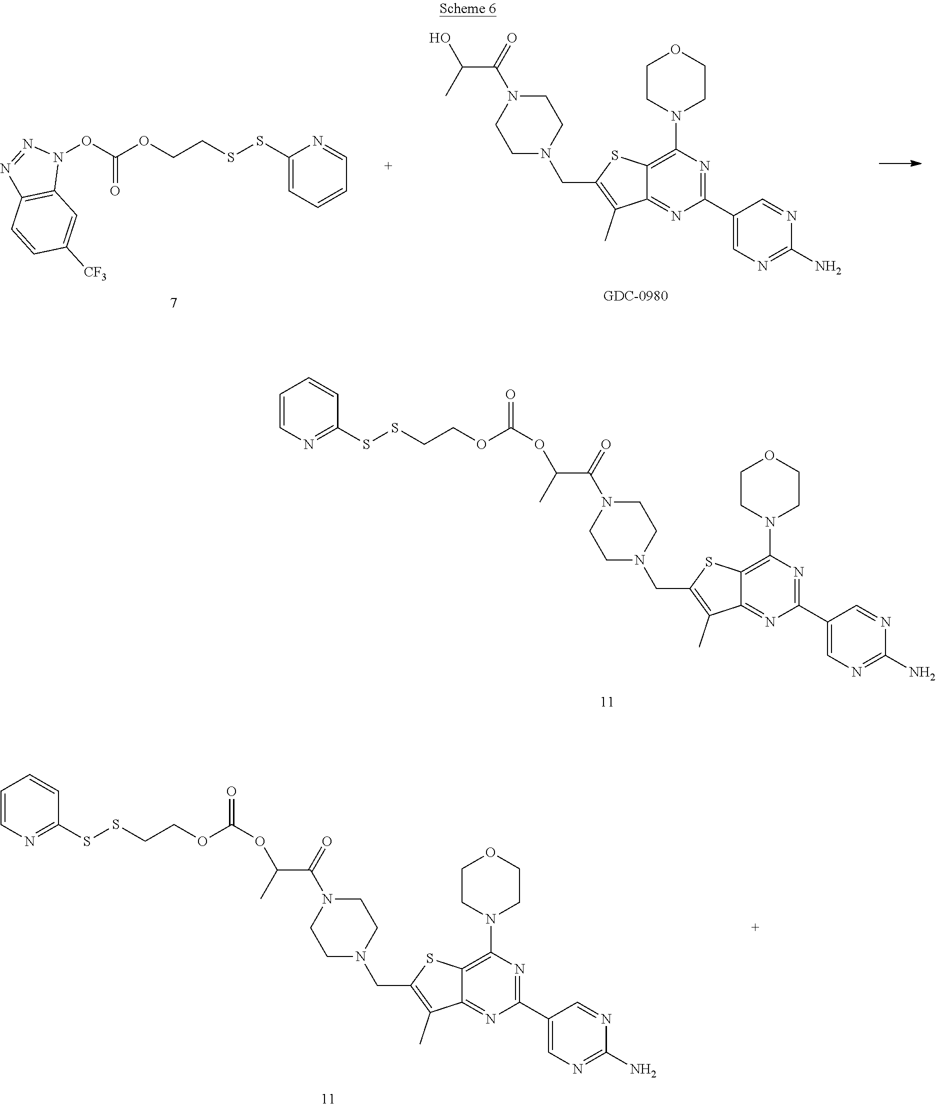 [SMILES: O=C(OCCSSc1ccccn1)On1nnc2ccc(C(F)(F)F)cc21 ; Cc1c(CN2CCN(C(=O)C(C)O)CC2)sc2c(N3CCOCC3)nc(-c3cnc(N)nc3)nc12 ; Cc1c(CN2CCN(C(=O)C(C)OC(=O)OCCSSc3ccccn3)CC2)sc2c(N3CCOCC3)nc(-c3cnc(N)nc3)nc12 ; Cc1c(CN2CCN(C(=O)C(C)OC(=O)OCCSSc3ccccn3)CC2)sc2c(N3CCOCC3)nc(-c3cnc(N)nc3)nc12]

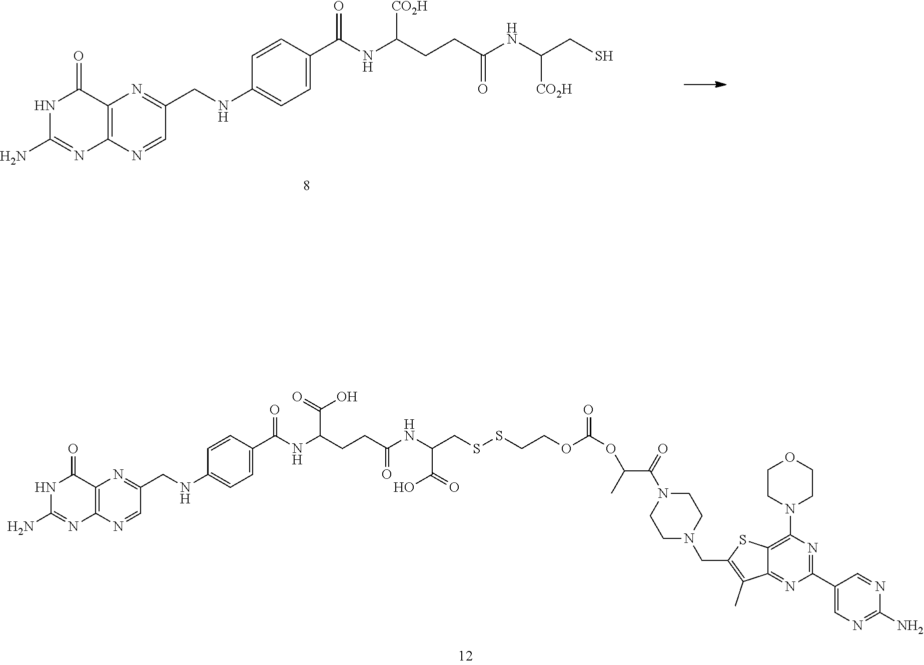 [SMILES: Nc1nc2ncc(CNc3ccc(C(=O)NC(CCC(=O)NC(CS)C(=O)O)C(=O)O)cc3)nc2c(=O)[nH]1 ; Cc1c(CN2CCN(C(=O)C(C)OC(=O)OCCSSCC(NC(=O)CCC(NC(=O)c3ccc(NCc4cnc5nc(N)[nH]c(=O)c5n4)cc3)C(=O)O)C(=O)O)CC2)sc2c(N3CCOCC3)nc(-c3cnc(N)nc3)nc12]

Heterobifunctional linker 7 (50 mg, 0.12 mmol) was added to a solution of GDC-0980 (5 mg, 0.01 mmol) and dimethylaminopyridine (5 mg, 0.03 mmol) in 4 mL of methylene chloride at RT under nitrogen atmosphere and the mixture was stirred at reflux temperature for 7 hours at which time TLC analysis of the mixture indicated >80% conversion. The mixture was concentrated and purified by column chromatography using 10% acetonitrile in methylene chloride as eluant. The pure product compound 9 was obtained as a light yellow solid. A solution of compound 8 (1 eq.) in DMSO was added in 3 portions at 20 min intervals to a solution of drug-linker intermediates compound 11 (1.0-1.5 eq.) in DMSO with dimethylaminopyridine (1 eq.). After 1-2 hours of stirring at room temperature under argon, LCMS analysis of the mixture indicated formation of the desired folate-drug conjugate compound 12 as the major product. The mixture was purified by preparative HPLC. LCMS: [M+H]⁺ m/z=1145.

### Example 7: Synthesis of FA-PBD Inhibitor (Compound 25)

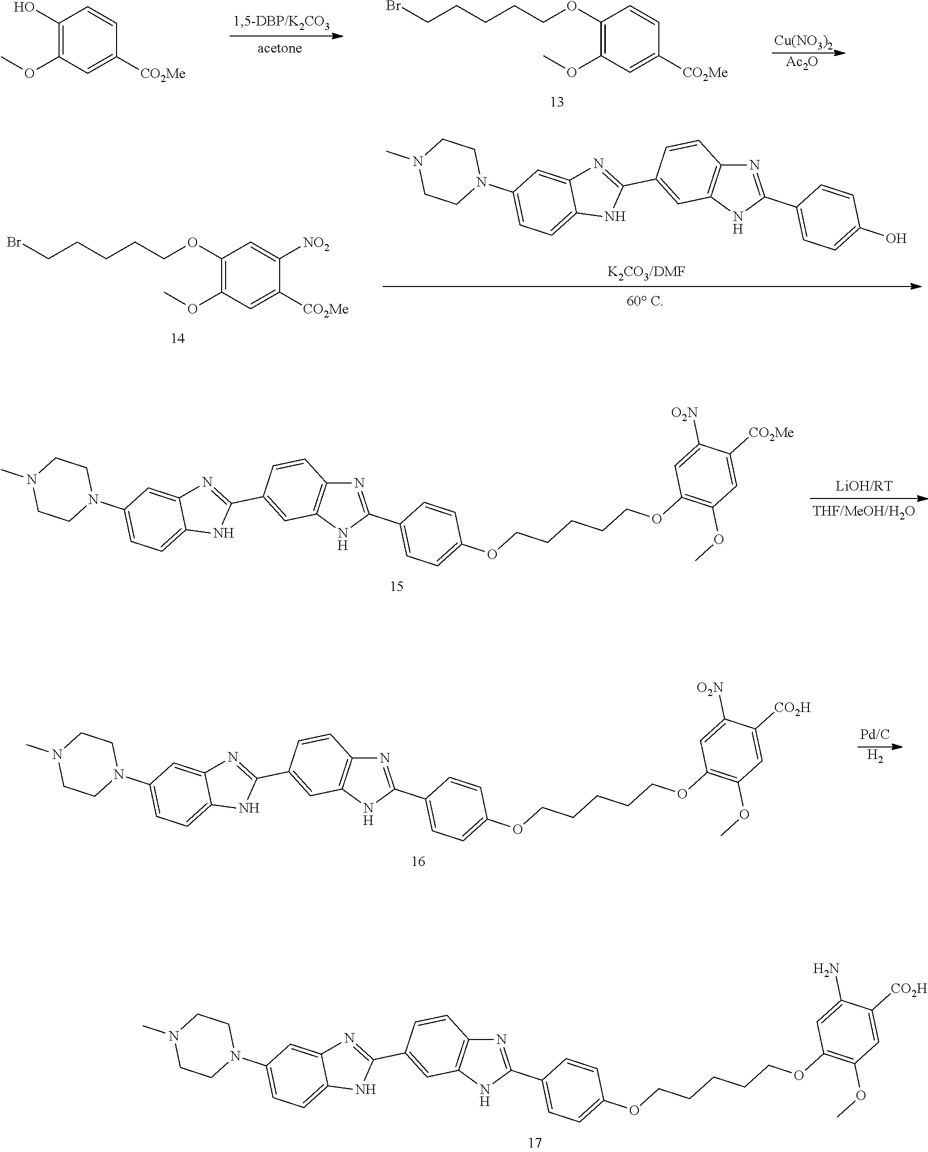

The phenol compound (2.20 g, 12.1 mmol) was dissolved in acetone (dried through a pad of Na₂SO₄, 48.4 mL) and to this solution was added 1,5-dibromopentane (49.4 mL, 36.3 mmol) and K₂CO₃ (6.69 g, 48.4 mmol). The reaction was heated to reflux under Ar for 6 hrs. The reaction was cooled to RT and the solid was filtered out. The filtrate was concentrated and purified with CombiFlash in 0-30% EtOAc/p-ether to obtained compound 13 (3.3893 g, yield 84.5%) as a solid. LCMS: [M+H]⁺ m/z=331. ¹H NMR (CDCl₃, δ in ppm): 7.65 (dd, J=8.5, 2.0 Hz, 1H), 7.54 (d, J=2.0 Hz, 1H), 6.86 (d, J=8.50 Hz, 1H), 4.08 (t, J=6.50 Hz, 2H), 3.91 (s, 3H), 3.89 (s, 3H), 3.44 (t, J=6.5 Hz, 2H), 1.95 (m, 4H), 1.65 (m, 2H).

Compound 13 (3.3893 g, 10.23 mmol) in Ac₂O (52 mL) was cooled to 0° C. and treated with Cu(NO₃).3H₂O (2.967 g, 12.28 mmol) by slow addition. The reaction was stirred at 0° C. for 1 hr then at RT for 2 hrs. After the reaction was completed, the reaction mixture was poured into ice water and stirred for 1 hr. The resultant precipitate was collected by filtration. The product was washed with water (3×) and air-dried as Compound 14 (3.7097 g, yield 96%). LCMS: [M+H]⁺ m/z=376. ¹H NMR (CDCl₃, δ in ppm): 7.41 (s, 1H), 7.05 (s, 1H), 4.08 (t, J=6.50 Hz, 2H), 3.94 (s, 3H), 3.89 (s, 3H), 3.42 (t, J=7.0 Hz, 2H), 1.93 (m, 4H), 1.63 (m, 2H).

The solution of Compound 14 (37.6 mg, 0.1 mmol) and Hochest dye (53.3 mg, 0.1 mmol) in DMF (1.5 mL) under Ar was treated with K₂CO₃ at rt. The reaction was heated to 60° C. and kept for overnight. Then the reaction was cooled to rt and the solid was filtered out. The residue was purified with Prep-HPLC (Mobile phase A: 50 mM NH₄HCO₃ buffer, pH 7.0; B=ACN. Method: 10-100 B % in 30 min.) to afford Compound 15 (13.1 mg, yield 18%). LCMS: [M+H]⁺ m/z=720.71.

Compound 15 (13.1 mg, 0.0182 mmol) was dissolved in THF/MeOH/H₂O (3/1/1, 0.2 mL) and treated with aq. LiOH solution (1 M, 36 μL) for 4 hrs at rt under Ar. Most of the solvent was removed in vacuo and the aqueous phase was acidified with concentrated HCl to pH 2-3, the precipitate was collected as solid (Compound 16, 12.8 mg, without purification) by filtration. The filtrate was washed with water (3×) and air dried for the next step. LCMS: [M+H]⁺ m/z=706.

Compound 16 (15.7 mg, 0.022 mmol) in MeOH (10 mL) was subjected to hydrogenation in a Parr shaker (10% wet Pd/C, 5% wt, 7.85 mg, H₂ 41 PSI) for 2 hrs. The product was isolated by filtration through a pad of celite. The solvent was removed in vacuo to give crude Compound 17. LCMS: [M+H]⁺ m/z=676.79.

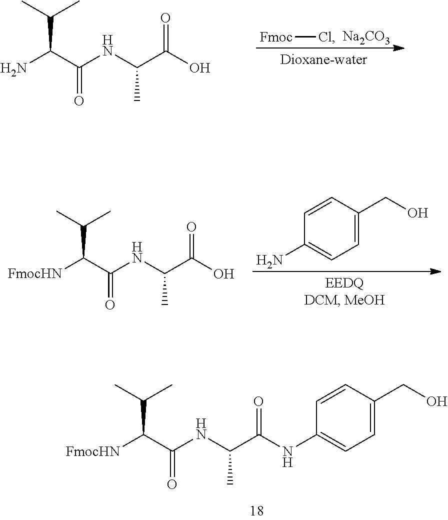

To a solution of Val-Ala-OH (1 g, 5.31 mM) in water (40 ml) was added Na₂CO₃ (1.42 g, 13.28 mM) and cooled to 0° C. before dioxane (40 mL) was added. A solution of Fmoc-Cl (1.44 g, 5.58 mM) in dioxane (40 mL) was added dropwise over 10 min at 0° C. The reaction mixture was stirred at 0° C. for 2 h, then allowed to stir at RT for 16 h. Dioxane was removed under vacuum, the reaction mixture diluted with water (450 mL), pH was adjusted to 2 using IN HCl and extracted with EtOAc (3×250 mL). The combined organic layers are washed with brine, dried over MgSO₄, filtered, concentrated under reduced pressure and dried to yield Fmoc-Val-Ala-OH. This product was suspended in dry DCM (25 ml), PABA (0.785 g, 6.38 mM) and EEDQ (1.971 g, 7.97 mM) are added. The resulting mixture was treated under Argon with methanol until a clear solution was obtained. The reaction was stirred overnight and filtered. The filtrate was washed with diethyl ether (4×) and dried under high vacuum to yield Compound 18 (1.85 g, 68%). ¹H NMR (500 MHz, CD₃OD): δ 7.79 (d, J₁=8.0 Hz, 2H), 7.65 (t, J₁=7.0 Hz, J₂=7.5 Hz, 2H), 7.54 (d, J₁=8.0 Hz, 2H), 7.38 (t, J₁=7.5 Hz, J₂=7.5 Hz, 2H), 7.33-7.24 (m, 4H), 4.54 (s, 2H), 4.48 (q, J₁=14.0 Hz, J₂=7.0 Hz, 1H), 4.42-4.32 (m, 2H), 4.22 (t, J₁=7.0 Hz, J₂=6.5 Hz, 1H), 3.94 (d, J₁=7.0 Hz, 1H), 2.07 (m, 1H), 1.43 (d, J₁=7.5 Hz, 3H), 0.97 (d, J₁=7.0 Hz, 3H), 0.95 (d, J₁=7.0 Hz, 3H); LCMS (ESI): (M+H)⁺=Calculated for C₃₀H₃₃N₃O₅, 516.24: found 516.24.

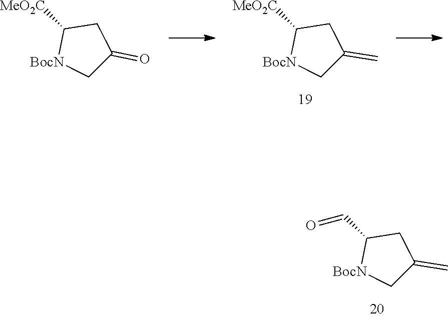

Compound 19. (S)-1-tert-butyl 2-methyl 4-oxopyrrolidine-1,2-dicarboxylate was converted to Compound 19 by Wittig reaction. Ph₃PCH₃Br (917.8 mg, 2.57 mmol) in THF (30 mL) was treated with KO^{t}Bu (1 M in THF, 2.57 μL, 2.57 mmol) at 0° C. by dropwise addition. The reaction was kept at ambient temperature for 2 h. Into the stirred solution was added the ketone (250 mg, 1.028 mmol) in THF 20 mL) at 0-10° C. The reaction was then stirred at ambient temperature overnight. The reaction was quenched with H₂O/EtOAc (1:1, 40 mL) and most THF was removed under reduced pressure. The aqueous phase was extracted with EtOAc (20 mL, 3×) and the organic phase was washed with H₂O, and brine sequentially and dried over anhydrous Na₂SO₄ and concentrated. The residue was purified with CombiFlash in 0-50% EtOAc/petroleum ether to give Compound 19 (77.2 mg, 31%). LCMS: [M-Boc+H]⁺ m/z=142.

Compound 20. (S)-1-tert-butyl 2-methyl 4-methylenepyrrolidine-1,2-dicarboxylate (353.2 mg, 1.46 mmol) in DCM/toluene (1:3, 9.8 mL) was treated with DIBAL (1 M in toluene, 2 eq, 2.92 mmol) dropwise at −78° C. under argon. The reaction was stirred at −78° C. for about 4 h. Then the reaction was quenched with addition of 60 μL of MeOH at −78° C. followed by 5% HCl (0.5 mL) and EtOAc (18 mL). The cold bath was removed and the reaction was stirred for 30 min. The EtOAc layer was separated and washed with brine, dried over anhydrous Na₂SO₄ and concentrated to give Compound 20.

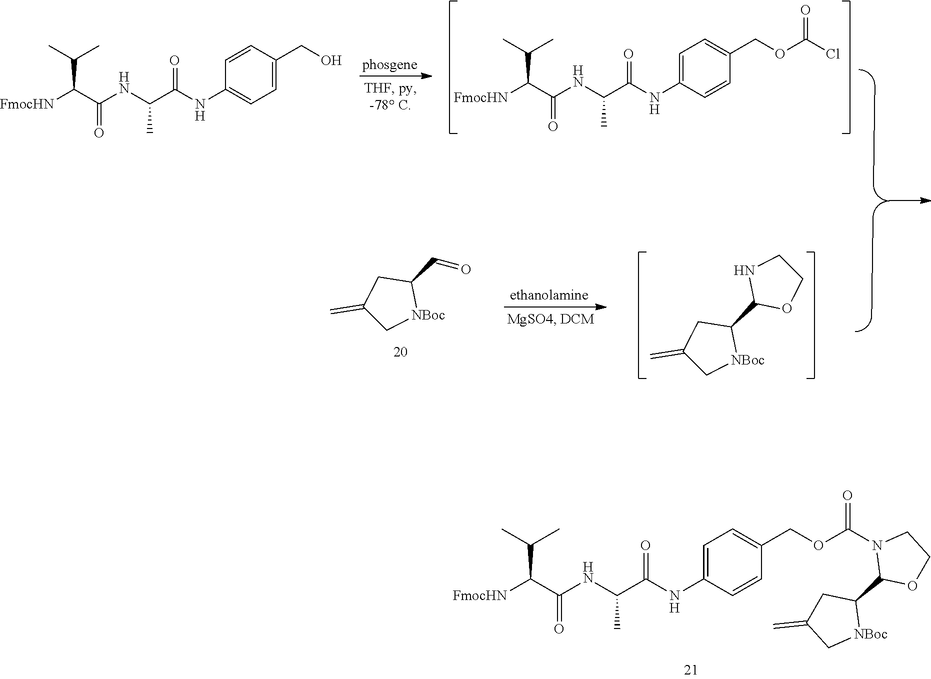

Compound 20 (550 mg, 2.6 mmol) was dissolved in DCM (10 mL), and MgSO₄ (3 g) was added followed by dropwise addition of ethanolamine (0.16 mL, 2.6 mmol) in DCM (10 mL). The reaction was stirred at rt for 1 hr. Filtration and concentration under vacuum gave the oxazoline intermediate. In another flask, Compound 18 (516 mg, 1.0 mmol) was dissolved in THF (40 mL) and pyridine was added (0.8 mL, 10 mmol). The solution was cooled to −78° C., and diphosgene (0.16 mL, 1.5 mmol) was added. The reaction was stirred at −78° C. for 1 h, DCM (20 mL) and a solution of oxazolidine intermediate was added dropwise. The reaction mixture was allowed to warm to −20° C. over several hours. LC-MS and TLC showed product formation. The reaction mixture was concentrated with silica gel and purified by flash chromatography (120 gold Redisep column, 0-100% EtOAc in petroleum ether) to give Compound 21 (0.59 g, 74%). LCMS (ESI): (M+H)⁺=Calculated for C₄₄H₅₃N₅O₉, 796.38; found 796.74.

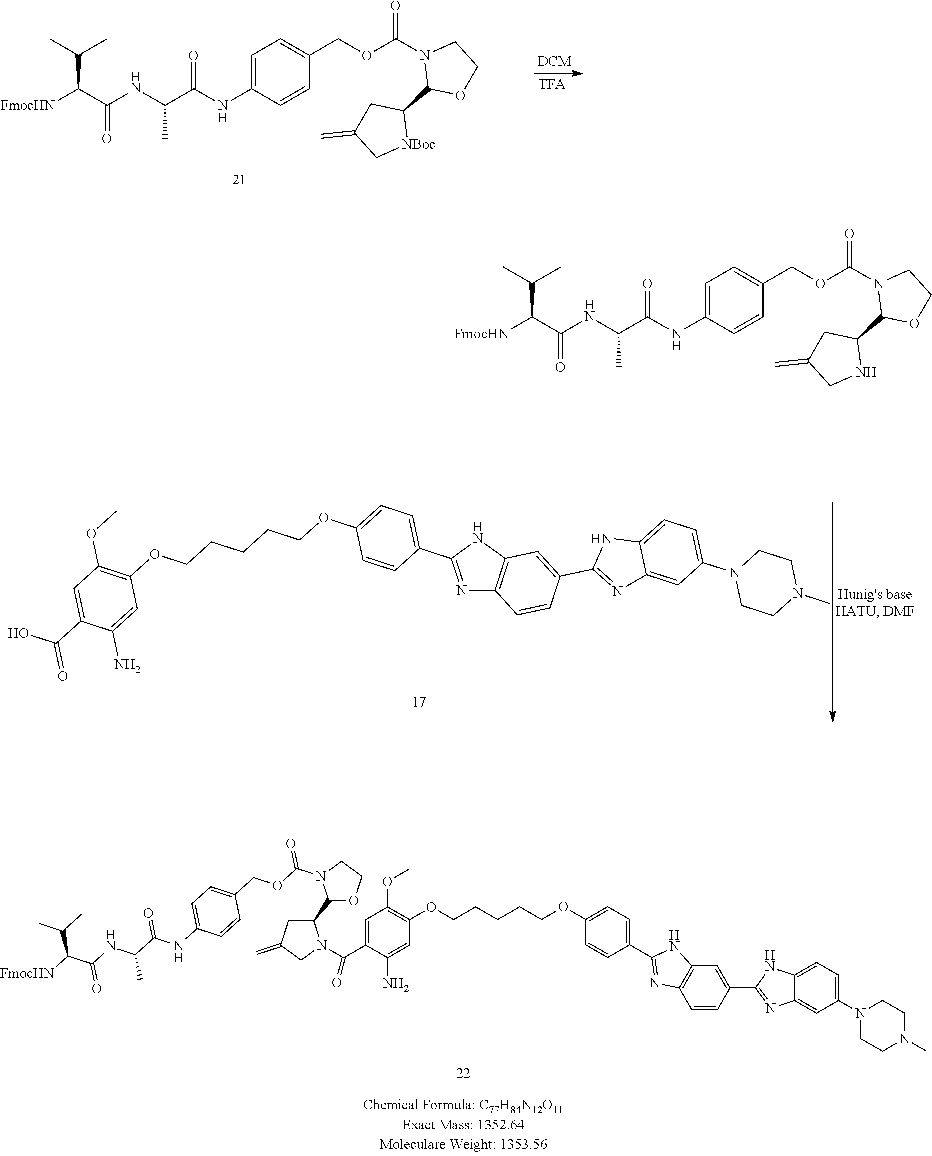

Compound 21 (101.0 mg, 0.127 mmol) was stirred in TFA/DCM (0.5 mL each) at rt for 30 min. LC-MS showed complete removal of Boc group. The reaction mixture was concentrated under high vacuum to remove TFA and DCM, re-dissolved in DMF (1.0 mL), and adjusted pH to 8-9 by adding Hunig's base (0.3 mL). Compound 17 (86.0 mg, 0.127 mmol) was added, followed by PyBoP (84 mg, 0.16 mmol) and the reaction was stirred at rt for 2 h. LC-MS at 90 min showed that the major peak had the desired product. The reaction mixture was loaded onto a silica gel cartridge and purified by flash chromatography (12 g gold, 0-30% MeOH/DCM) to give desired product, Compound 22 (140 mg, 81%). LCMS (ESI): (M+H)⁺=Calculated for C₇₇H₈₄N₁₂O₁₁, 1353.64; found 1354.18.

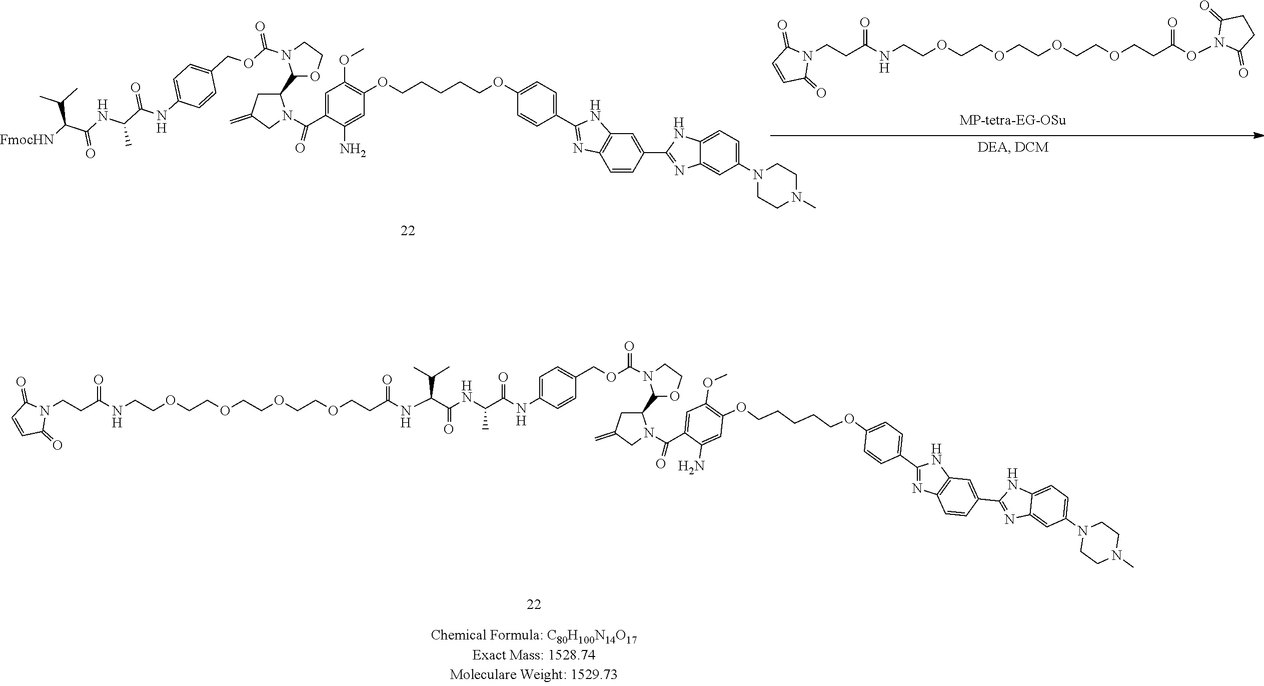

Compound 22 (140 mg, 0.10 mmol) was dissolved in DEA/DCM (12/18 mL) and stirred at rt for 30 min. LC-MS showed complete removal of Fmoc group. The reaction mixture was concentrated under high vacuum to remove excess diethylamine and re-dissolved in DCM (5 mL). Commercially available α-Maleimidopropionyl-ω-succinimidyl-4(ethylene glycol) (Mal-PEG₄-NHS) (62 mg, 0.12 mmol) was added and the reaction was stirred at rt for 1 hr. The reaction mixture was concentrated, redissolved in DMSO and loaded directly to HPLC column and purified by preparative HPLC (C18 column, 5-80% ACN/pH7 buffer) giving desired product Compound 23 (55.8 mg, 36%). LCMS: [M+2H]²⁺ m/z=Calculated for C₈₀H₁₀₀N₁₄O₁₇, 765.37; found 765.74.

N¹⁰-TFA Protected Compound 24. N¹⁰-TFA Protected Compound 24 was prepared according to the following process.

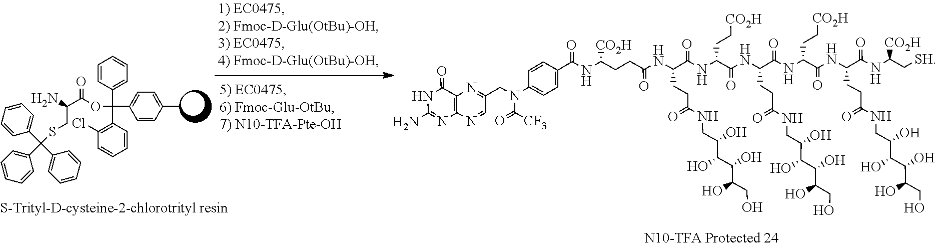 [SMILES: Cc1ccc(C(OC(=O)[C@H](N)CSC(c2ccccc2)(c2ccccc2)c2ccccc2)(c2ccccc2)c2ccccc2Cl)cc1 ; Nc1nc2ncc(CN(C(=O)C(F)(F)F)c3ccc(C(=O)N[C@@H](CCC(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CCC(=O)O)C(=O)N[C@@H](CCC(=O)NC[C@H](O)[C@@H](O)[C@H](O)[C@H](O)CO)C(=O)N[C@H](CS)C(=O)O)C(=O)O)cc3)nc2c(=O)[nH]1]

Compound 24 was prepared as described in WO2014/062679. Compound 24 was prepared according to the following process.

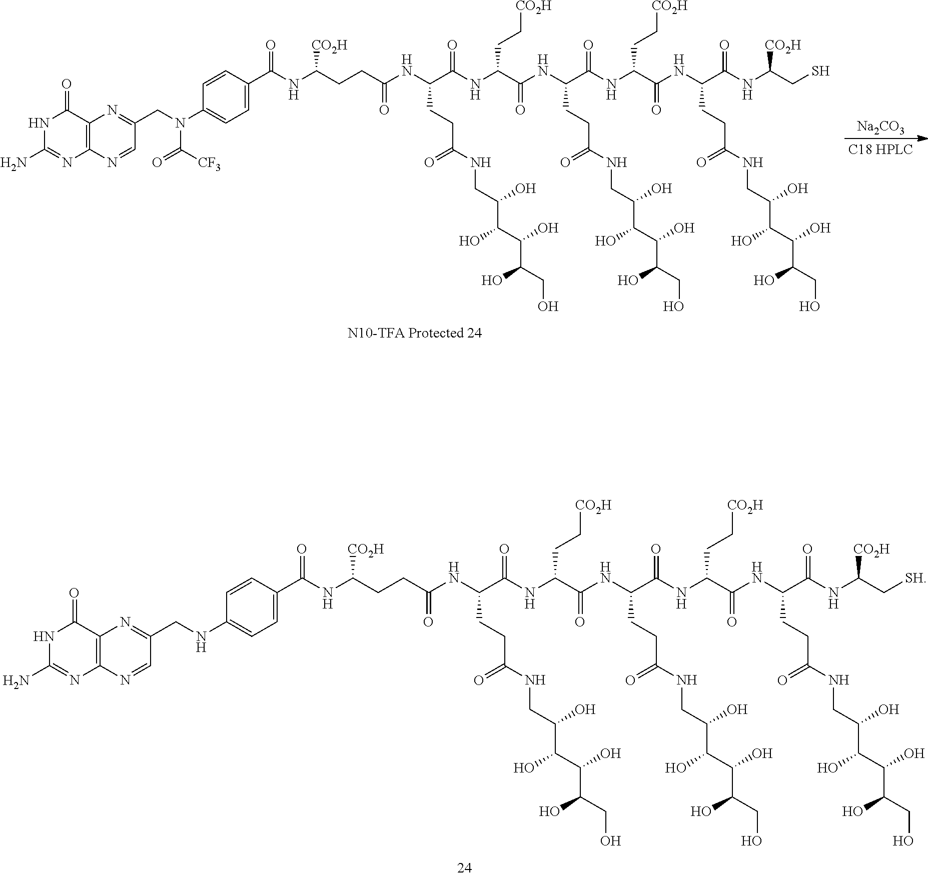

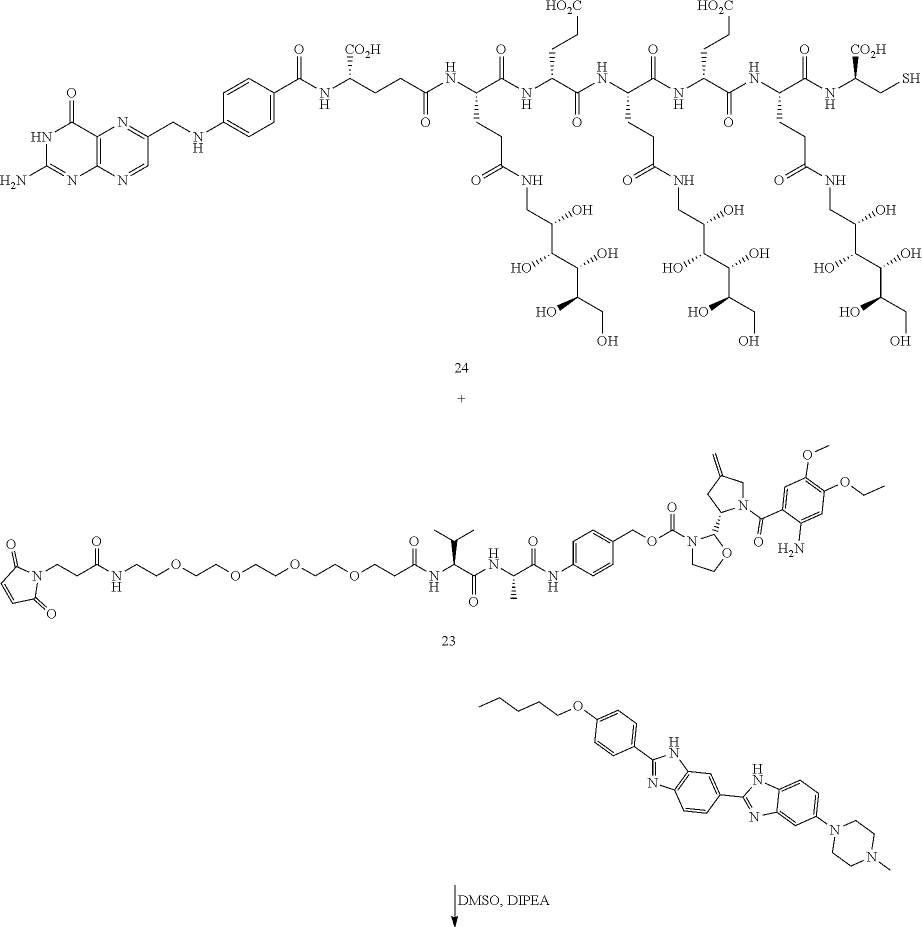

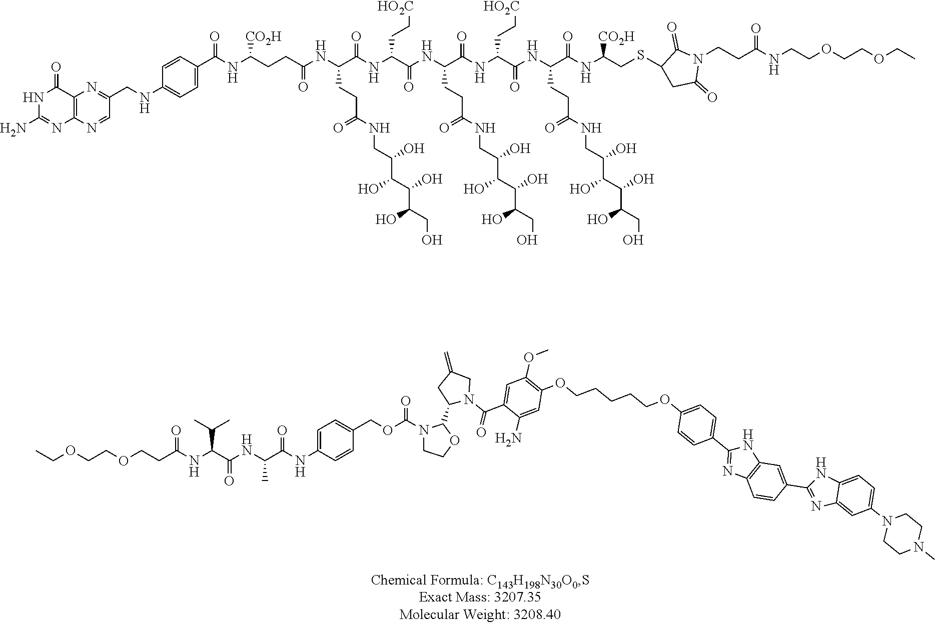

Compound 24 (9.85 mg, 0.006 mmol) was stirred in DMSO (2 mL) until dissolved. DIPEA (50 uL) was added, followed by Compound 23 (6.24 mg, 0.004 mmol) in DMSO (2 mL). The reaction was stirred at RT for 50 min. LC-MS analysis at 10 min showed complete conversion. The reaction mixture was directly loaded on a prep-HPLC column and purified (10-100% MeCN/Ammonium bicarbonate, pH 7 buffer) to give desired product Example 25 (5.5 mg, 42%). ¹H NMR (500 MHz, DMSO-D₆+D₂O) (selected data): δ 8.60 (s, 1H), 8.44-8.08 (m*, 1H), 8.07 (d, J=8.5 Hz, 2H), 8.06-7.84 (m*, 2H), 7.80-7.57 (m*, 2H), 7.57 (d, J=8 Hz, 2H), 7.51 (d, J=6.5 Hz, 2H), 7.44 (m*, 1H), 7.22 (m*, 2H), 7.08 (d, J=8 Hz, 2H), 6.93 (d, J=8.5 Hz, 1H), 6.60 (d, J=8.5 Hz, 2H), 6.33 (s, 1H), 4.95 (m*, 4H), 4.45 (m*, 3H); LCMS: [M+4H]⁴⁺ m/z=Calculated for C₁₄₅H₁₉₈N₃₀O₅₁S, 803.34; found 803.80.

### Comparative Example 1

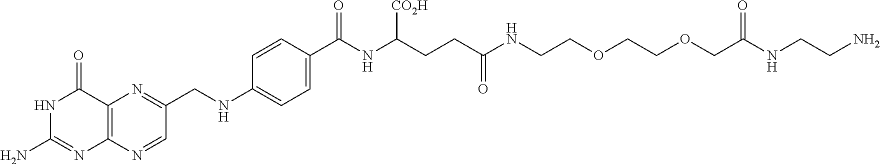 [SMILES: NCCNC(=O)COCCOCCNC(=O)CCC(NC(=O)c1ccc(NCc2cnc3nc(N)[nH]c(=O)c3n2)cc1)C(=O)O]

(also referred to herein as competitor or competition)

What is claimed is:

1. A compound of the formula: 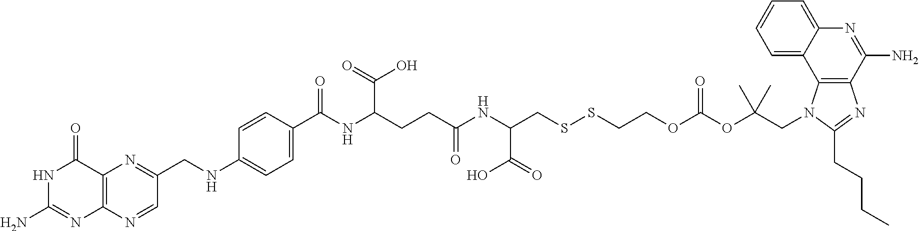 [SMILES: CCCCc1nc2c(N)nc3ccccc3c2n1CC(C)(C)OC(=O)OCCSSCC(NC(=O)CCC(NC(=O)c1ccc(NCc2cnc3nc(N)[nH]c(=O)c3n2)cc1)C(=O)O)C(=O)O]
or a pharmaceutically acceptable salt thereof.

2. A pharmaceutical composition comprising a compound of claim 1 and at least one of pharmaceutically-acceptable carriers, adjuvants, diluents, excipients, and vehicles.

3. A dosage form comprising a compound of claim 1.

4. A dosage form comprising the pharmaceutical composition of claim 2.

5. The dosage form of claim 3 or 4, wherein the compound is present in an effective amount.

6. The dosage form of claim 5, wherein the effective amount is from about 0.5 mg/m² to about 6.0 mg/m².

7. The dosage form of claim 3 or 4, wherein the dosage form is a parenteral dosage form.

8. The dosage form of claim 7, wherein the parenteral dosage form is selected from an intradermal dosage form, a subcutaneous dosage form, an intramuscular dosage form, an intraperitoneal dosage form, an intravenous dosage form, and an intrathecal dosage form.
